(19) United States
(12) Patent Application Publication
(10) Pub. No.: US 2024/0217977 A1
(43) Pub. Date: Jul. 4, 2024

(54) DIAZEPANONE-FUSED PYRIMIDINE COMPOUNDS, COMPOSITIONS AND MEDICINAL APPLICATIONS THEREOF

(71) Applicant: Blueprint Medicines Corporation, Cambridge, MA (US)

(72) Inventors: Gurulingappa Hallur, Menlo Park, CA (US); Anjali Pandey, Menlo Park, CA (US); Bruce Roth, Menlo Park, CA (US); Tracy Saxton, Menlo Park, CA (US)

(21) Appl. No.: 18/284,943

(22) PCT Filed: Mar. 30, 2022
(86) PCT No.: PCT/US2022/022592

§ 371 Date: Sep. 29, 2023

Related U.S. Application Data
(60) Provisional application No. 63/168,896, filed on Mar. 31, 2021.

(51) Int. Cl. C07D 487/04; A61K 31/551

(52) U.S. Cl. CPC C07D 487/04; A61K 31/551

(57) ABSTRACT

The present disclosure relates to a class of diazepanone-fused pyrimidine compounds of Formula I, their stereoisomers, tautomers, pharmaceutically acceptable salts, polymorphs, solvates, and hydrates thereof. The present disclosure also relates to a process of preparation of these diazepanone-fused pyrimidine compounds, and to pharmaceutical compositions containing them.

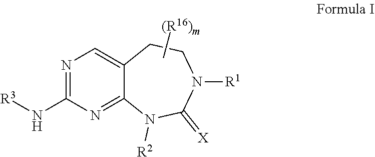

### CROSS-REFERENCE TO RELATED APPLICATIONS

This application claims priority to U.S. Provisional Application No. 63/168,896, filed Mar. 31, 2021. The entire contents of the aforementioned application are incorporated herein by reference.

### BACKGROUND OF THE INVENTION

Lung cancer accounts for the greatest number of cancer deaths, and approximately 85% of lung cancer cases are non-small cell lung cancer (NSCLC). The development of targeted therapies for lung cancer has primarily focused on tumors displaying specific oncogenic drivers, namely mutations in epidermal growth factor receptor (EGFR) and anaplastic lymphoma kinase (ALK). Three generations of tyrosine kinase inhibitors (TKIs) have been developed for cancers with the most frequently observed EGFR mutations, however, other oncogenic drivers in the EGFR family of receptor tyrosine kinases have received less research and development focus and several oncogenic drivers, including insertions in the exon 20 gene of EGFR, have no currently approved therapeutics to treat their cancers.

The mutation, amplification and/or overexpression of human epidermal growth factor receptor 2 (HER2), another member of the human epidermal growth factor receptor family of receptor tyrosine kinases, has been implicated in the oncogenesis of several cancers, including lung, breast, ovarian, and gastric cancers. Although targeted therapies such as trastuzumab and lapatinib have shown clinical efficacy especially in breast tumors, their utility in lung cancer has been limited. It is likely that this variation is due to tissue-specific factors, including the low potency of kinase inhibitors like lapatinib for the mutagenic alterations in HER2 that are observed in the lung cancer patient population, including insertions in the exon 20 gene of HER2.

Given that many patients with mutations in EGFR and HER2 do not derive clinical benefit from currently available therapies against these targets, there remains a significant unmet need for the development of novel therapies for the treatment of cancers associated with EGFR and HER2 mutations.

### SUMMARY OF THE INVENTION

In one aspect, provided herein is a compound of Formula I:

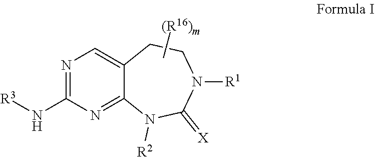

or a pharmaceutically acceptable salt thereof, wherein:
X is O or S;
R¹ is —(C(R⁴)₂)ₙR⁵, wherein R⁵ is substituted with 0, 1, or 2 R^{5′};
   n is 0, 1, 2, or 3;
   each R⁴ is independently hydrogen, alkyl, halo, haloalkyl, hydroxy, alkoxy, or heteroalkyl;
   R⁵ is cycloalkyl, heterocycloalkyl, aryl, heteroaryl;
   each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂ alkyl, —S(═O)₂aryl, —S(═O)₂heteroaryl, or alkoxy;
each R⁶ is independently alkyl, cycloalkyl, aryl, or heteroaryl;
R² is aryl, heteroaryl, cycloalkyl, or heterocycloalkyl, wherein the aryl, heteroaryl, cycloalkyl, or heterocycloalkyl is substituted with at least one R⁷ and 0, 1, or 2 R⁸;
   each R⁷ is independently

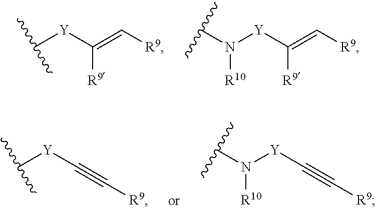

Y is —C(═O)—, —S(═O)—, or —S(═O)₂—;
R⁹ and R^{9′} are independently hydrogen, halo, alkyl, haloalkyl, cycloalkyl, heteroalkyl, or (alkyl)heterocycloalkyl;
R¹⁰ is hydrogen, alkyl, haloalkyl, or cycloalkyl;
each R⁸ is independently aryl, heteroaryl, alkyl, cycloalkyl, heterocycloalkyl, halo, heteroalkyl, haloalkyl, cyano, hydroxy, amino, —N(R¹¹)₂, —S(═O)₂alkyl, —S(═O)₂aryl, —S(═O)₂heteroaryl, or alkoxy;
   each R¹¹ is independently alkyl, cycloalkyl, aryl, or heteroaryl;
R³ is alkyl, aryl, heteroaryl, cycloalkyl, or heterocycloalkyl substituted with 0, 1, 2, or 3 R¹²;
   each R¹² is independently aryl, heteroaryl, alkyl, heteroalkyl, haloalkyl, halo, cyano, alkoxy, heterocycloalkyl, —N(R¹³)₂, —S(═O)₂NH₂, —S(═O)₂NMe₂, —S(═O)₂alkyl, —S(═O)₂aryl, —S(═O)₂heteroaryl, or cycloalkyl, wherein the aryl, heteroaryl, heterocycloalkyl, or cycloalkyl are each independently substituted with 0, 1, or 2 R¹⁴;
      each R¹³ is independently alkyl, cycloalkyl, aryl, or heteroaryl
      each R¹⁴ is independently aryl, heteroaryl, alkyl, cycloalkyl, heterocycloalkyl, halo, heteroalkyl, haloalkyl, cyano, hydroxy, amino, —N(R¹⁵)₂, —S(═O)₂alkyl, —S(═O)₂aryl, —S(═O)₂heteroaryl, or alkoxy;
      each R¹⁵ is independently alkyl, cycloalkyl, aryl, or heteroaryl;
each R¹⁶ is independently aryl, heteroaryl, alkyl, heteroalkyl, haloalkyl, halo, cyano, hydroxy, amino, alkoxy, heterocycloalkyl, —N(R¹⁷)₂, —S(═O)₂NH₂, —S(═O)₂NMe₂, —S(═O)₂alkyl, —S(═O)₂aryl, —S(═O)₂heteroaryl, or cycloalkyl, wherein the aryl, heteroaryl, heterocycloalkyl, or cycloalkyl are each independently substituted with 0, 1, or 2 R¹⁸;
   each R¹⁷ is independently alkyl, cycloalkyl, aryl, or heteroaryl
   each R¹⁸ is independently aryl, heteroaryl, alkyl, cycloalkyl, heterocycloalkyl, halo, heteroalkyl, haloalkyl, cyano, hydroxy, amino, —N(R¹⁹)₂, —S(═O)₂alkyl, —S(═O)₂aryl, —S(═O)₂heteroaryl, or alkoxy;
      each R¹⁹ is independently alkyl, cycloalkyl, aryl, or heteroaryl; and
m is 0, 1, 2, 3, or 4.

In some embodiments, n is 0 or 1.

In some embodiments, R⁵ is cyclopropyl, phenyl, naphthyl, anthracenyl, phenanthrenyl, pyridyl, pyrimidinyl, pyrazolyl, or imidazolyl. In some embodiments, R⁵ is unsubstituted. In some embodiments, R⁵ is substituted with 1 or 2 R^{5′}.

In some embodiments, each R⁴ is independently hydrogen, alkyl, halo, haloalkyl, or alkoxy. In some embodiments, each R⁴ is independently hydrogen, methyl, ethyl, n-propyl, iso-propyl, n-butyl, iso-butyl, sec-butyl, tert-butyl, fluoro, chloro, trifluoromethyl, trifluoroethyl, pentafluoroethyl, methoxy, ethoxy, or trifluoromethoxy. In some embodiments, each R⁴ is independently hydrogen, methyl, fluoro, trifluoromethyl, methoxy, or trifluoromethoxy.

In some embodiments, each R^{5′} is independently aryl, heteroaryl, alkyl, heterocycloalkyl, halo, cyano, hydroxy, —N(R⁶)₂, or alkoxy. In some embodiments, each R^{5′} is independently phenyl, pyrrolyl, imidazolyl, thiazolyl, isothiazolyl, oxazolyl, isoxazolyl, pyridinyl, pyrimidinyl, methyl, ethyl, tert-butyl, pyrrolidinyl, piperidinyl, piperazinyl, morpholinyl, fluoro, chloro, cyano, hydroxy, —N(R⁶)₂, methoxy, ethoxy, or trifluoromethoxy. In some embodiments, each R^{5′} is independently phenyl, imidazolyl, pyridinyl, methyl, tert-butyl, pyrrolidinyl, morpholinyl, fluoro, cyano, hydroxy, —N(R⁶)₂, or methoxy.

In some embodiments, each R⁶ is independently alkyl or aryl. In some embodiments, each R⁶ is independently methyl, ethyl, iso-propyl, tert-butyl, phenyl, or naphthyl. In some embodiments, each R⁶ is independently methyl or phenyl.

In some embodiments, X is S. In some embodiments, X is O.

In some embodiments, R² is monocyclic. In some embodiments, R² is phenyl, cyclopropyl, cyclobutyl, cyclopentyl, cyclohexyl, pyrrolyl, imidazolyl, pyrazolyl, triazolyl, tetrazolyl, thiazolyl, isothiazolyl, oxazolyl, isoxazolyl, pyridinyl, pyrimidinyl, pyridazinyl, pyrazinyl, or triazinyl. In some embodiments, R² is phenyl, cyclohexyl, or pyrrolyl.

In some embodiments, R⁷ is

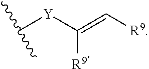

In embodiments, R⁷ is

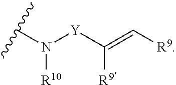

In some embodiments, Y is —C(═O)—. In some embodiments, Y is —S(═O)₂—.

In some embodiments, R⁹ and R^{9′} are independently hydrogen, halo, alkyl, heteroalkyl, haloalkyl, or (alkyl)heterocycloalkyl. In some embodiments, R⁹ and R^{9′} are independently hydrogen, fluoro, chloro, methyl, hydroxyethyl, methoxyethyl, methoxymethyl, hydroxymethyl, dimethylaminomethyl, 1-piperidinylmethyl, 1-morpholinylmethyl, or fluoromethyl. In some embodiments, R⁹ and R^{9′} are independently hydrogen, fluoro, chloro, hydroxyethyl, or methoxymethyl.

In some embodiments, R¹⁰ is hydrogen, methyl, ethyl n-propyl, iso-propyl, n-butyl, sec-butyl, tert-butyl, trifluoromethyl, or cyclopropyl. In some embodiments, R¹⁰ is hydrogen or methyl.

In some embodiments, R² is substituted with 1 or 2 R⁸.

In some embodiments, each R⁸ is independently methyl, ethyl, n-propyl, iso-propyl, n-butyl, iso-butyl, sec-butyl, tert-butyl, fluoro, chloro, heteroalkyl, cyano, hydroxy, amino, —N(R¹¹)₂, methoxy, ethoxy, or trifluoromethoxy. In some embodiments, each R⁸ is independently methyl, ethyl, iso-propyl, tert-butyl, fluoro, chloro, —N(R¹¹)₂, hydroxyethyl, methoxyethyl, or cyano.

In some embodiments, each R¹¹ is independently alkyl or aryl. In some embodiments, each R¹¹ is independently methyl, ethyl, n-propyl, iso-propyl, n-butyl, iso-butyl, sec-butyl, tert-butyl, phenyl, naphthyl, anthracenyl, or phenanthrenyl. In some embodiments, each R¹¹ is independently methyl, ethyl, iso-propyl, tert-butyl, phenyl, or naphthyl. In some embodiments, each R¹¹ is independently methyl or phenyl.

In some embodiments, R² is unsubstituted.

In some embodiments, R³ is phenyl, cyclopentyl, tetrahydropyranyl, oxetanyl, pyrrolyl, imidazolyl, pyrazolyl, triazolyl, tetrazolyl, indolyl, indazolyl, benzimidazolyl, azaindolyl, thiazolyl, isothiazolyl, oxazolyl, isoxazolyl, pyridinyl, pyrimidinyl, pyridazinyl, pyrazinyl, triazinyl, quinolinyl, isoquinolinyl, quinoxalinyl, quinazolinyl, cinnolinyl, or naphthyridinyl. In some embodiments, R³ is phenyl, cyclopentyl, tetrahydropyranyl, oxetanyl, imidazolyl, triazolyl, indolyl, indazolyl, thiazolyl, isothiazolyl, or pyridinyl.

In some embodiments, R³ is selected from:

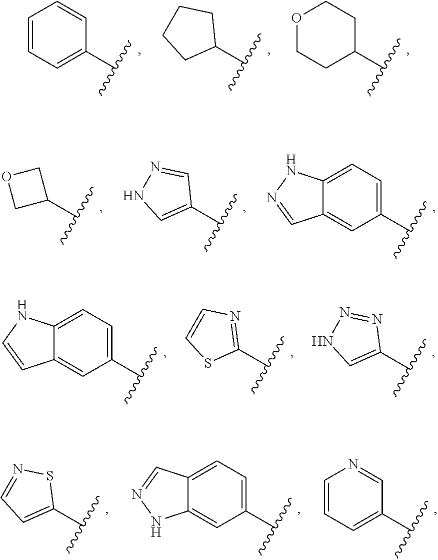

wherein R³ is substituted with 0 to 3 R¹².

In some embodiments, R³ is selected from:

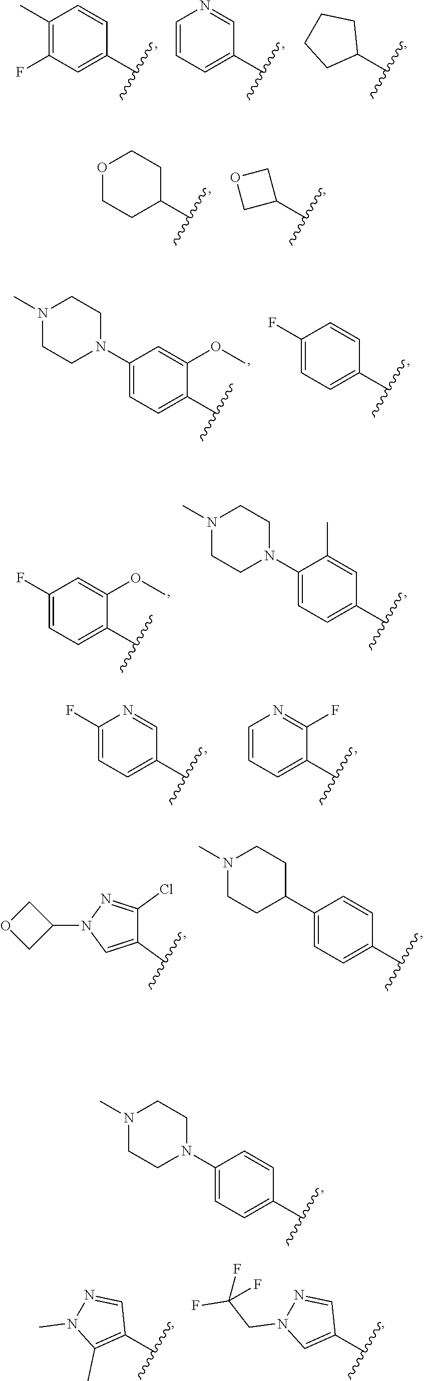

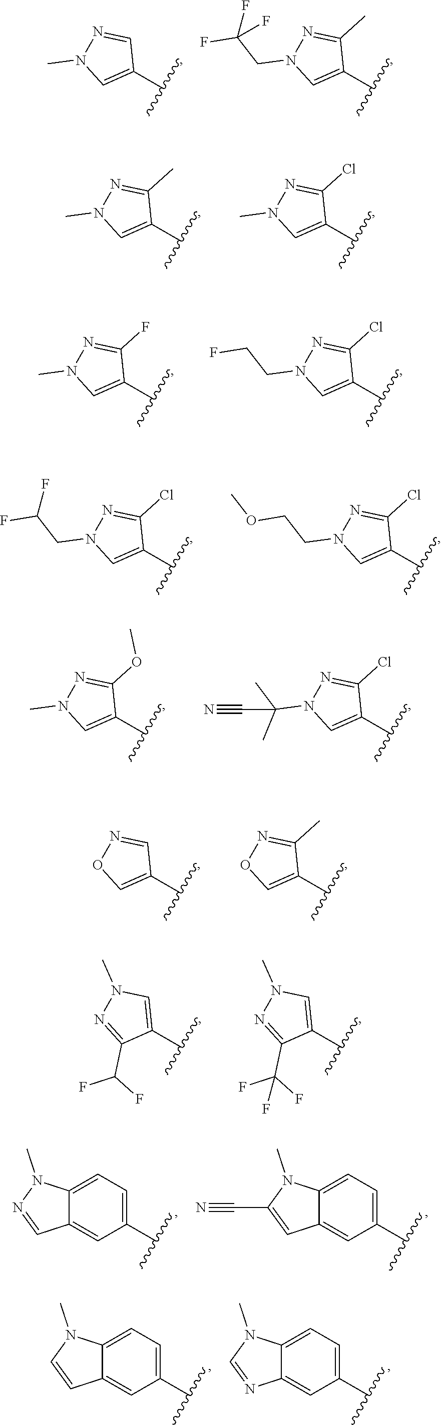

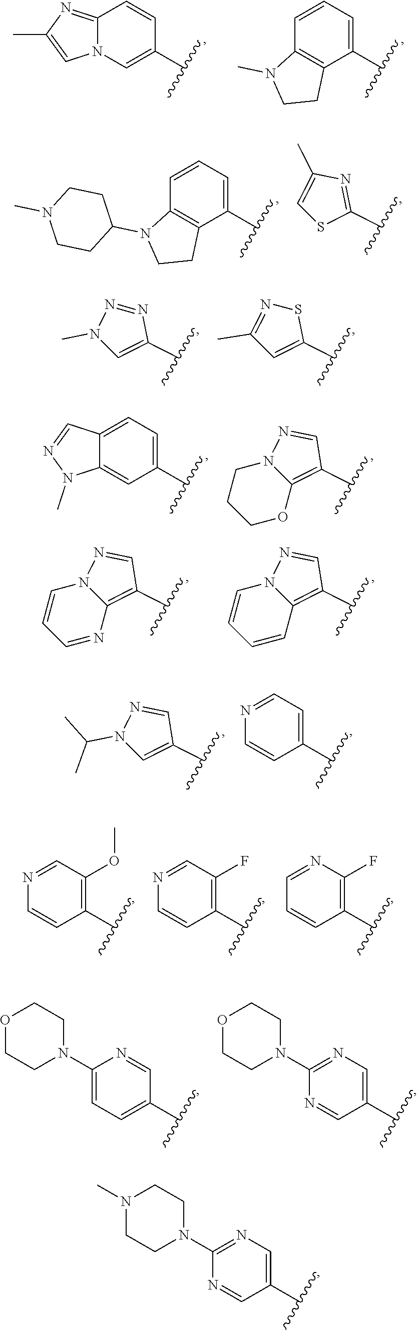

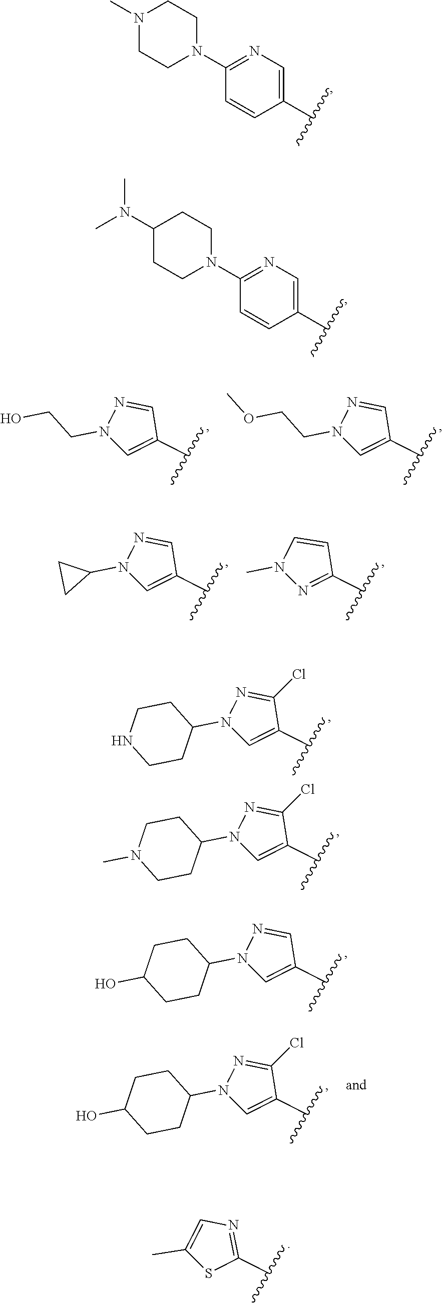

In some embodiments, R³ is selected from:

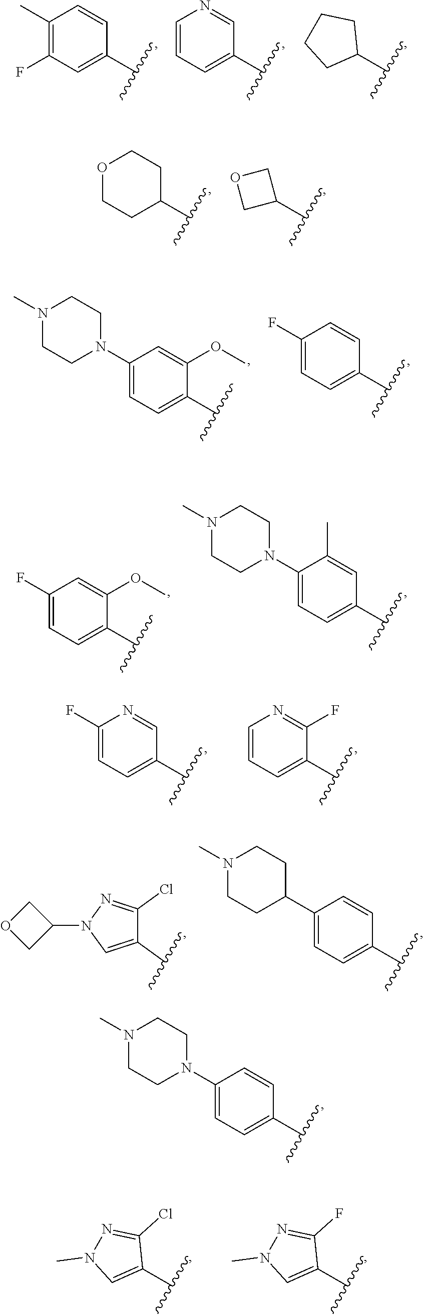

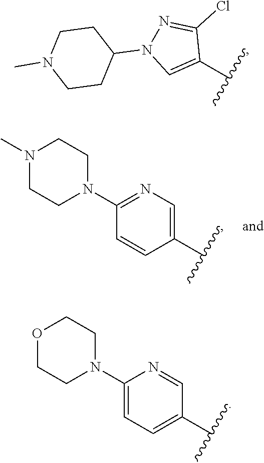 [SMILES: *c1cn(C2CCN(C)CC2)nc1Cl ; *c1ccc(N2CCN(C)CC2)nc1 ; *c1ccc(N2CCOCC2)nc1]

In some embodiments, R³ is unsubstituted. In some embodiments, R³ is substituted with at least 1 R¹². In some embodiments, R³ is substituted with at least 2 R¹².

In some embodiments, each R¹² is independently aryl, heteroaryl, alkyl, heteroalkyl, haloalkyl, halo, cyano, alkoxy, heterocycloalkyl, —N(R¹³)₂, —S(═O)₂NH₂, or cycloalkyl. In some embodiments, each R¹² is independently methyl, ethyl, n-propyl, iso-propyl, n-butyl, iso-butyl, sec-butyl, tert-butyl, hydroxyethyl, methoxyethyl, trifluoromethyl, trifluoroethyl, pentafluoroethyl, fluoro, chloro, cyano, methoxy, azetidinyl, oxetanyl, pyrrolidinyl, imidazolidinyl, tetrahydrofuranyl, piperidinyl, piperazinyl, tetrahydropyranyl, morpholinyl, —N(R¹³)₂, cyclopropyl, cyclobutyl, cyclopentyl, or cyclohexyl. In some embodiments, each R¹² is independently methyl, iso-propyl, tert-butyl, hydroxyethyl, methoxyethyl, trifluoromethyl, trifluoroethyl, fluoro, chloro, cyano, methoxy, oxetanyl, piperidinyl, piperazinyl, morpholinyl, or cyclopropyl. In some embodiments, each R¹² is independently methyl, fluoro, chloro, methoxy, oxetanyl, piperidinyl, piperazinyl, or morpholinyl. In some embodiments, each R¹² is independently methyl or chloro.

In some embodiments, each R¹³ is independently alkyl or cycloalkyl. In some embodiments, each R¹³ is independently methyl, ethyl, n-propyl, iso-propyl, n-butyl, iso-butyl, sec-butyl, tert-butyl, cyclopropyl, cyclobutyl, cyclopentyl, or cyclohexyl. In some embodiments, each R¹³ is independently methyl, ethyl, iso-propyl, tert-butyl, cyclopropyl, cyclopentyl, or cyclohexyl. In some embodiments, each R¹³ is independently methyl, cyclopropyl, or cyclohexyl.

In some embodiments, the aryl, heteroaryl, heterocycloalkyl, or cycloalkyl of R¹² is unsubstituted. In some embodiments, the aryl, heteroaryl, heterocycloalkyl, or cycloalkyl of R¹² is substituted with 1 or 2 R¹⁴.

In some embodiments, each R¹⁴ is independently alkyl, cycloalkyl, heterocycloalkyl, halo, cyano, —N(R¹⁵)₂, or alkoxy. In some embodiments, each R¹⁴ is independently methyl, ethyl, n-propyl, iso-propyl, n-butyl, iso-butyl, sec-butyl, tert-butyl, cyclopropyl, cyclobutyl, cyclopentyl, cyclohexyl, azetidinyl, oxetanyl, pyrrolidinyl, imidazolidinyl, tetrahydrofuranyl, piperidinyl, piperazinyl, tetrahydropyranyl, morpholinyl, fluoro, chloro, cyano, —N(R¹⁵)₂ methoxy, ethoxy, or trifluoromethoxy. In some embodiments, each R¹⁴ is independently methyl, ethyl, iso-propyl, tert-butyl, pyrrolidinyl, piperidinyl, morpholinyl, fluoro, chloro, —N(R¹⁵)₂, or methoxy.

In some embodiments, each R¹⁵ is independently alkyl or cycloalkyl. In some embodiments, each R¹⁵ is methyl, ethyl, n-propyl, iso-propyl, n-butyl, iso-butyl, sec-butyl, tert-butyl, cyclopropyl, cyclobutyl, cyclopentyl, or cyclohexyl. In some embodiments, each R¹⁵ is independently methyl, ethyl, iso-propyl, tert-butyl, cyclopropyl, cyclopentyl, or cyclohexyl. In some embodiments, each R¹⁵ is independently methyl, cyclopropyl, or cyclohexyl.

In some embodiments, the compound of Formula I is selected from:

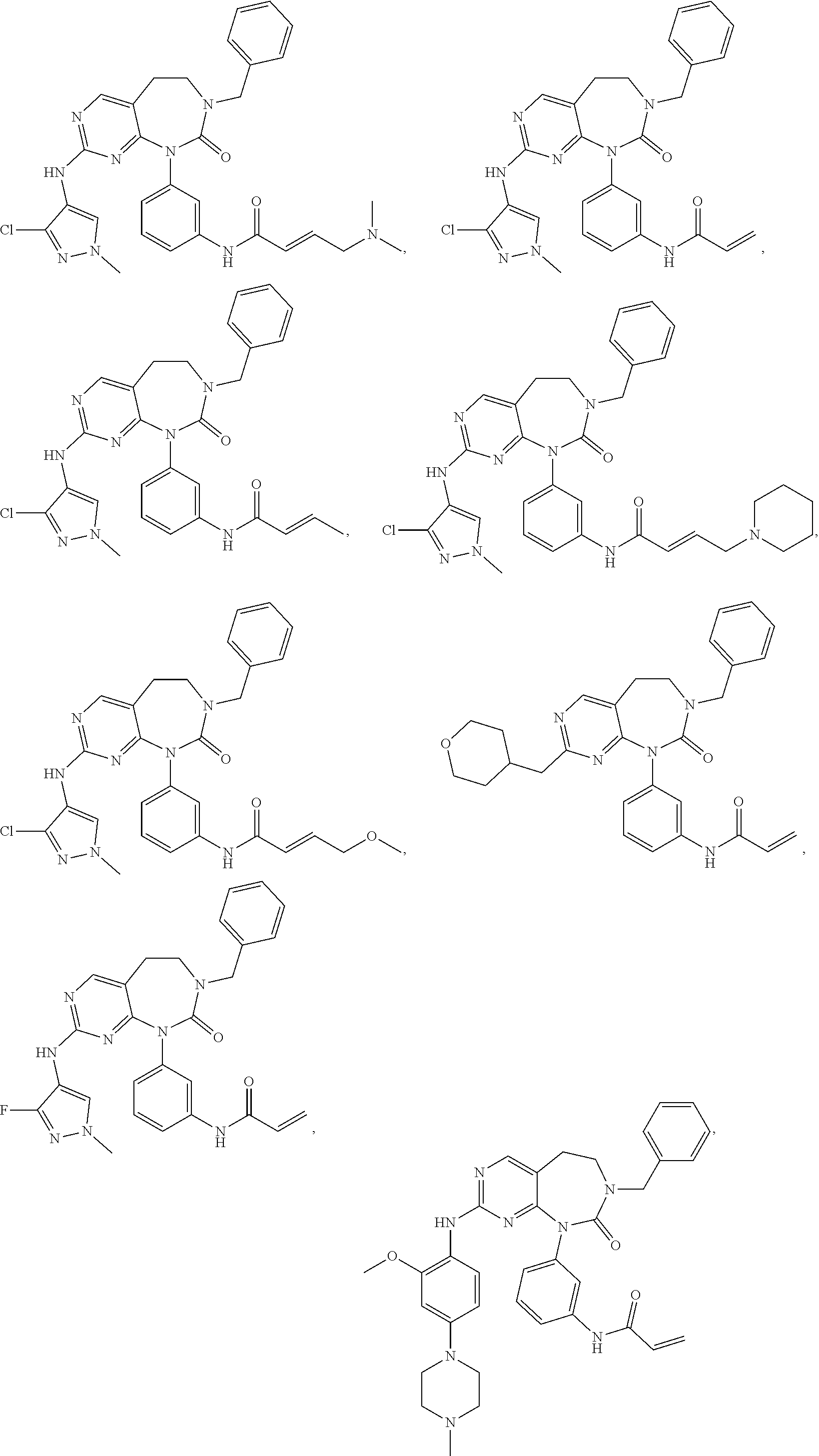

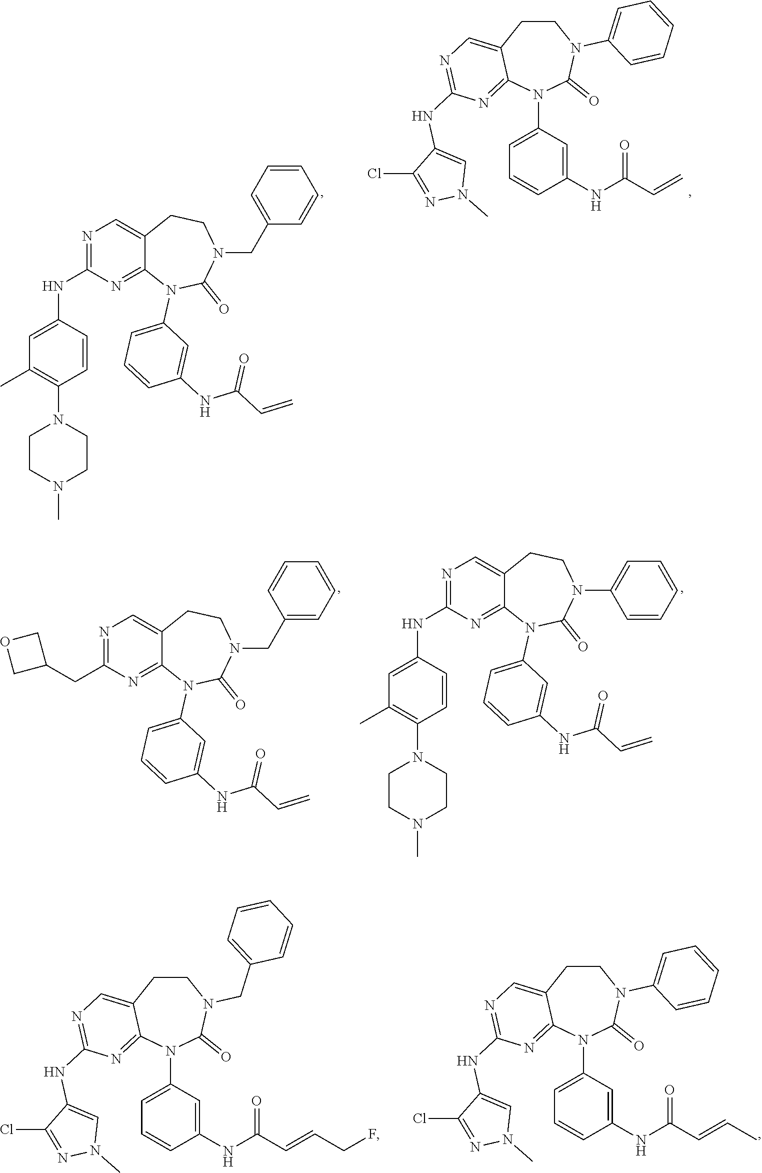 [SMILES: C=CC(=O)Nc1cccc(N2C(=O)N(Cc3ccccc3)CCc3cnc(Nc4ccc(N5CCN(C)CC5)c(C)c4)nc32)c1 ; C=CC(=O)Nc1cccc(N2C(=O)N(c3ccccc3)CCc3cnc(Nc4cn(C)nc4Cl)nc32)c1 ; C=CC(=O)Nc1cccc(N2C(=O)N(Cc3ccccc3)CCc3cnc(CC4COC4)nc32)c1 ; C=CC(=O)Nc1cccc(N2C(=O)N(c3ccccc3)CCc3cnc(Nc4ccc(N5CCN(C)CC5)c(C)c4)nc32)c1 ; Cn1cc(Nc2ncc3c(n2)N(c2cccc(NC(=O)/C=C/CF)c2)C(=O)N(Cc2ccccc2)CC3)c(Cl)n1 ; C/C=C/C(=O)Nc1cccc(N2C(=O)N(c3ccccc3)CCc3cnc(Nc4cn(C)nc4Cl)nc32)c1]

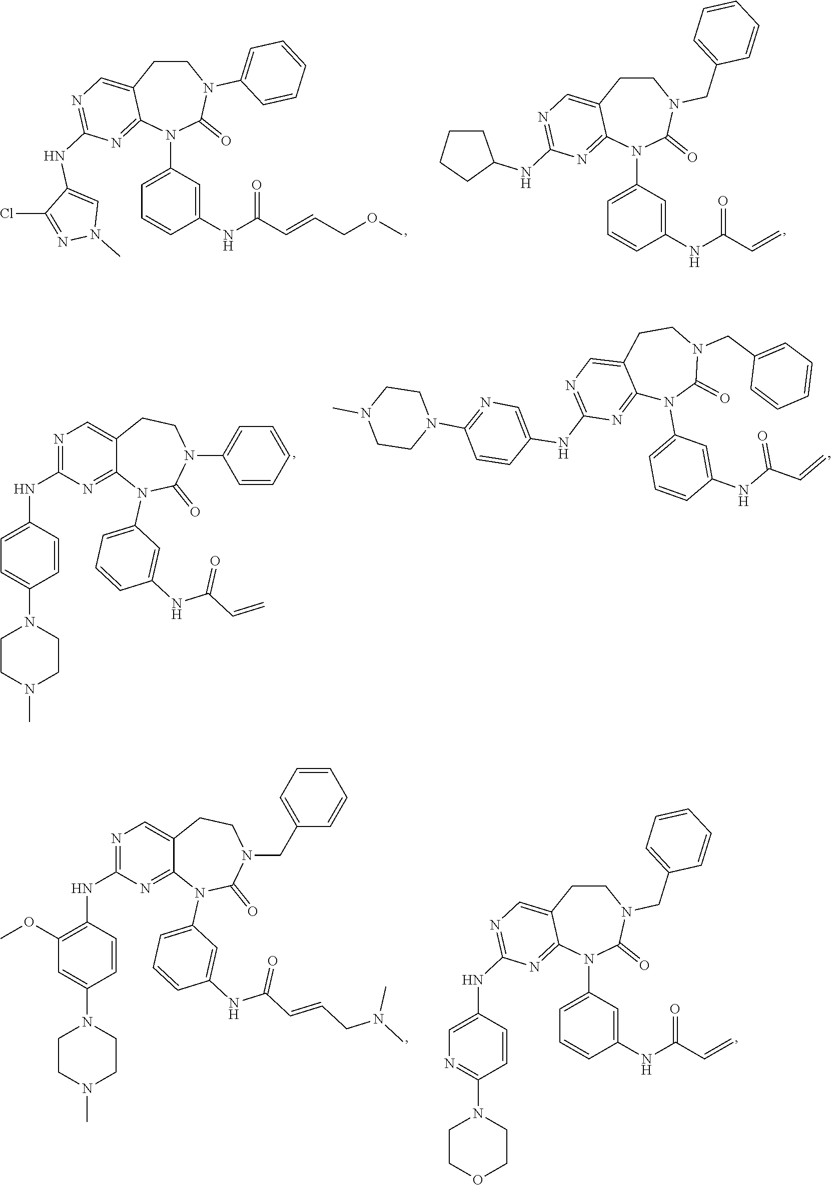

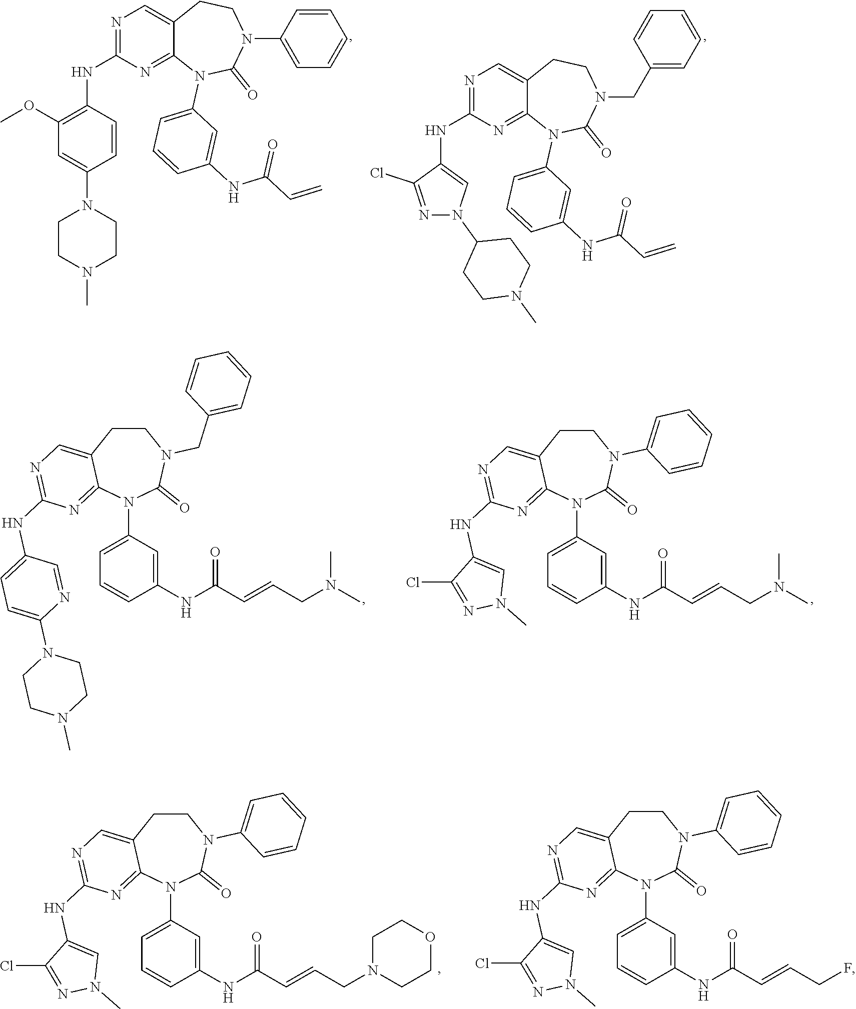 [SMILES: C=CC(=O)Nc1cccc(N2C(=O)N(c3ccccc3)CCc3cnc(Nc4ccc(N5CCN(C)CC5)cc4OC)nc32)c1 ; C=CC(=O)Nc1cccc(N2C(=O)N(Cc3ccccc3)CCc3cnc(Nc4cn(C5CCN(C)CC5)nc4Cl)nc32)c1 ; CN(C)C/C=C/C(=O)Nc1cccc(N2C(=O)N(Cc3ccccc3)CCc3cnc(Nc4ccc(N5CCN(C)CC5)nc4)nc32)c1 ; CN(C)C/C=C/C(=O)Nc1cccc(N2C(=O)N(c3ccccc3)CCc3cnc(Nc4cn(C)nc4Cl)nc32)c1 ; Cn1cc(Nc2ncc3c(n2)N(c2cccc(NC(=O)/C=C/CN4CCOCC4)c2)C(=O)N(c2ccccc2)CC3)c(Cl)n1 ; Cn1cc(Nc2ncc3c(n2)N(c2cccc(NC(=O)/C=C/CF)c2)C(=O)N(c2ccccc2)CC3)c(Cl)n1]

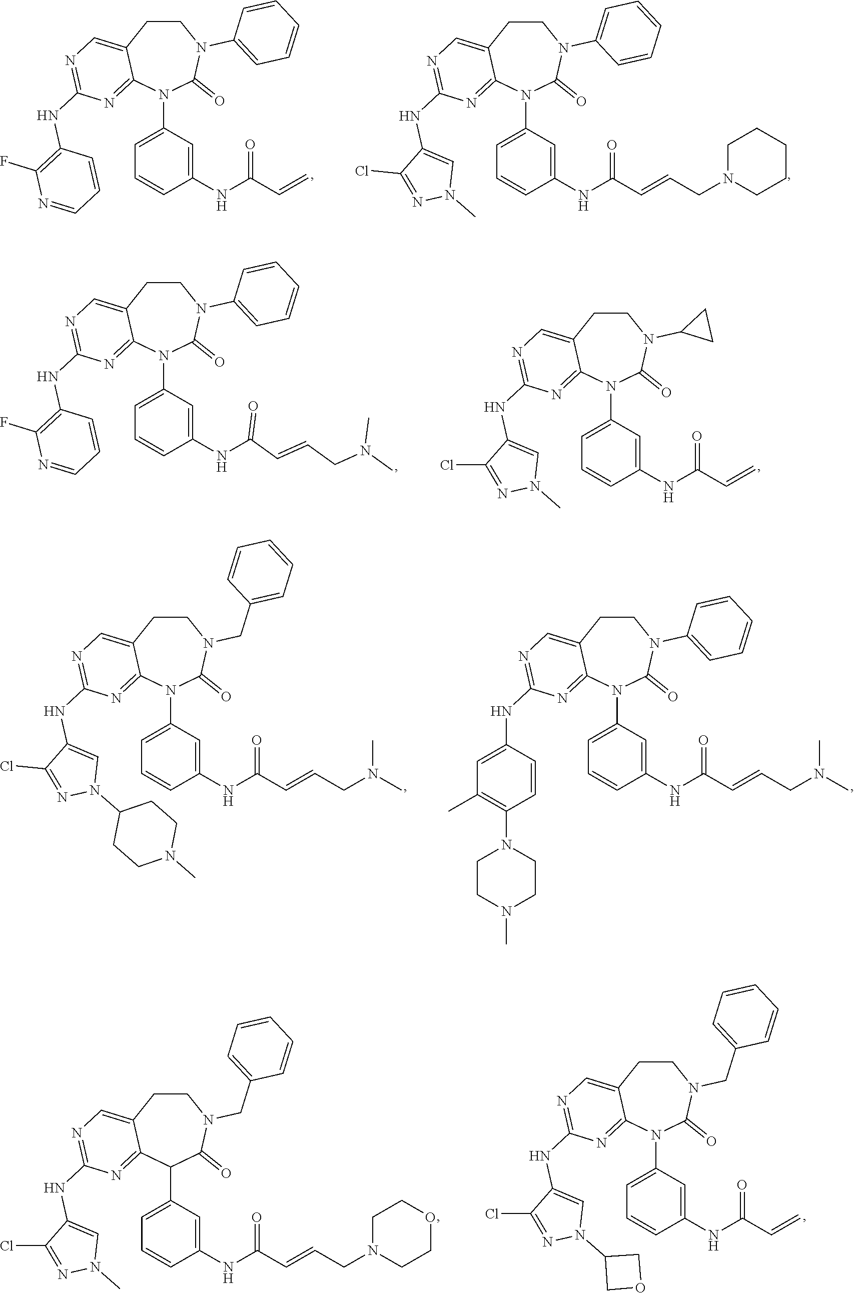

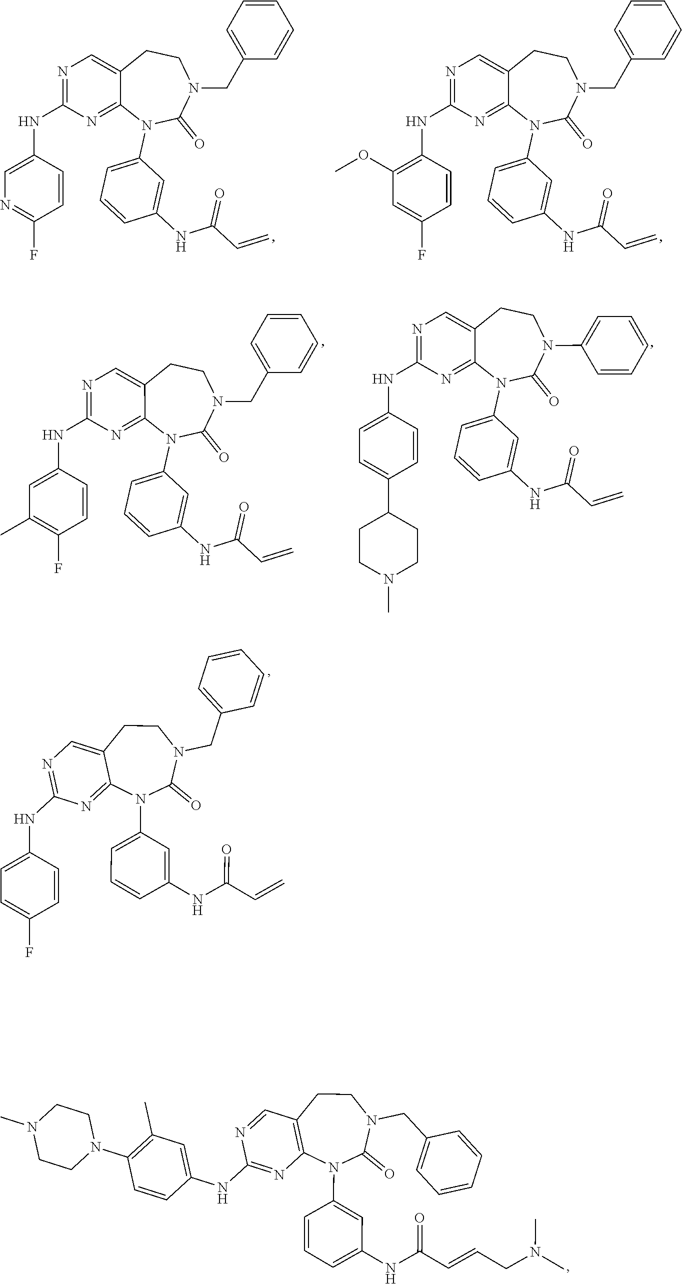

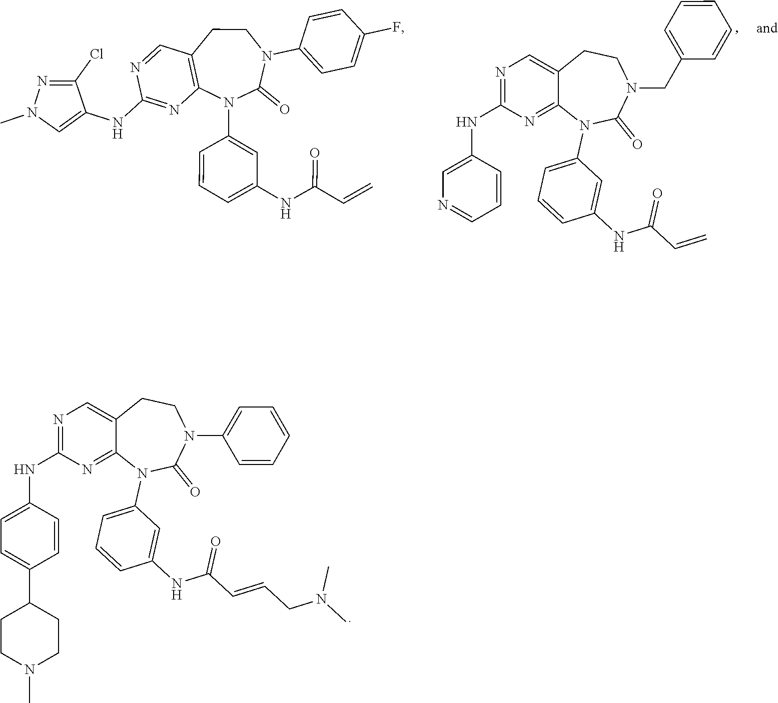 [SMILES: C=CC(=O)Nc1cccc(N2C(=O)N(c3ccc(F)cc3)CCc3cnc(Nc4cn(C)nc4Cl)nc32)c1 ; C=CC(=O)Nc1cccc(N2C(=O)N(Cc3ccccc3)CCc3cnc(Nc4cccnc4)nc32)c1 ; CN(C)C/C=C/C(=O)Nc1cccc(N2C(=O)N(c3ccccc3)CCc3cnc(Nc4ccc(C5CCN(C)CC5)cc4)nc32)c1]

In another aspect, provided herein is a pharmaceutical composition comprising a compound of Formula I, or a pharmaceutically acceptable salt thereof, and a pharmaceutically acceptable carrier.

In another aspect, provided herein is a method of inhibiting an epidermal growth factor receptor (EGFR) family kinase mutant in a subject in need thereof, comprising administering to the subject a therapeutically effective amount of a compound of Formula I, or a pharmaceutically acceptable salt thereof.

In another aspect, provided herein is a method of inhibiting a human epidermal growth factor receptor 2 (HER2) mutant in a subject in need thereof, comprising administering to the subject a therapeutically effective amount of a compound of Formula I, or a pharmaceutically acceptable salt thereof. In some embodiments, the HER2 mutant comprises an insertion in exon 20, an in-frame deletion and insertion in exon 20, a substitution in the extracellular domain, an extracellular truncation, or a substitution in exon 30. In some embodiments, the HER2 mutant is A775ins_G776insYVMA, A775_G776insSVMA, A775_G776insVVMA, G776del insVC, G776del insLC, G776del insAV, G776del insAVGC, S310F, S310Y, p95, V842I, or P780_Y781insGSP.

In another aspect, provided herein is a method of inhibiting an epidermal growth factor receptor (EGFR) mutant in a subject in need thereof, comprising administering to the subject a therapeutically effective amount of a compound of Formula I, or a pharmaceutically acceptable salt thereof.

In another aspect, provided herein is a method of inhibiting a drug-resistant epidermal growth factor receptor (EGFR) mutant in a subject in need thereof, comprising administering to the subject a therapeutically effective amount of a compound of Formula I, or a pharmaceutically acceptable salt thereof. In some embodiments, the drug-resistant EGFR mutant is del19/T790M EGFR or L858R/T790M EGFR.

In another aspect, provided herein is a method of inhibiting human epidermal growth factor receptor 2 (HER2) in a subject in need thereof, comprising administering to the subject a therapeutically effective amount of a compound of Formula I, or a pharmaceutically acceptable salt thereof, wherein the compound exhibits greater inhibition of a HER2 mutant relative to wild-type EGFR. In some embodiments, the HER2 mutant comprises an insertion in exon 20, an in-frame deletion and insertion in exon 20, a substitution in the extracellular domain, an extracellular truncation, or a substitution in exon 30. In some embodiments, the HER2 mutant is A775ins_G776insYVMA, A775_G776insSVMA, A775_G776insVVMA, G776del insVC, G776del insLC, G776del insAV, G776del insAVGC, S310F, S310Y, p95, V842I, or P780_Y781insGSP.

In another aspect, provided herein is a method of inhibiting epidermal growth factor receptor (EGFR) in a subject in need thereof, comprising administering to the subject a therapeutically effective amount of a compound of Formula I, or a pharmaceutically acceptable salt thereof, wherein the compound exhibits greater inhibition of an EGFR mutant relative to wild-type EGFR.

In some embodiments, the EGFR mutant comprises a substitution in exon 18, a deletion in exon 19, a substitution in exon 20, an insertion in exon 20, a mutation in the extracellular domain, or a substitution in exon 21. In some embodiments, the EGFR mutant is del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, D770_N771insSVD EGFR, H773insNPH EGFR, A767_dupASV EGFR, or 773insAH EGFR. In some embodiments, the EGFR mutant is del19/T790M EGFR or L858R/T790M EGFR.

In another aspect, provided herein is a method of treating a disease or disorder associated with an epidermal growth factor receptor (EGFR) family kinase in a subject in need thereof, comprising administering to the subject a therapeutically effective amount of a compound of Formula I, or a pharmaceutically acceptable salt thereof.

In some embodiments, the disease or disorder in the subject comprises a HER2 mutation. In some embodiments, the HER2 mutation comprises an insertion in exon 20, an in-frame deletion and insertion in exon 20, a substitution in the extracellular domain, an extracellular truncation, or a substitution in exon 30. In some embodiments, the HER2 mutation is A775ins_G776insYVMA, A775_G776insSVMA, A775_G776insVVMA, G776del insVC, G776del insLC, G776del insAV, G776del insAVGC, S310F, S310Y, p95, V842I, or P780_Y781insGSP.

In some embodiments, the disease or disorder in the subject comprises an EGFR mutation. In some embodiments, the EGFR mutation comprises a substitution in exon 18, a deletion in exon 19, a substitution in exon 20, an insertion in exon 20, a mutation in the extracellular domain, or a substitution in exon 21. In some embodiments, the EGFR mutation is del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, D770_N771insSVD EGFR, H773insNPH EGFR, A767_dupASV EGFR, or 773insAH EGFR. In some embodiments, the EGFR mutation is del19/T790M EGFR or L858R/T790M EGFR.

In another aspect, provided herein is a method of treating one or more cancer cells in a subject in need thereof, comprising administering to the subject a therapeutically effective amount of a compound of Formula I, or a pharmaceutically acceptable salt thereof.

In another aspect, provided herein is a method of treating cancer in a subject in need thereof, comprising administering to the subject a therapeutically effective amount of a compound of Formula I, or a pharmaceutically acceptable salt thereof.

In some embodiments, the cancer is selected from bladder cancer, prostate cancer, breast cancer, cervical cancer, colorectal cancer, endometrial cancer, gastric cancer, glioblastoma, head and neck cancer, lung cancer, and non-small cell lung cancer. In some embodiments, the cancer is selected from non-small cell lung cancer, prostate cancer, head and neck cancer, breast cancer, colorectal cancer, and glioblastoma.

In some embodiments, the cancer in the subject comprises a HER2 mutation. In some embodiments, the HER2 mutation comprises an insertion in exon 20, an in-frame deletion and insertion in exon 20, a substitution in the extracellular domain, an extracellular truncation, or a substitution in exon 30. In some embodiments, the HER2 mutation is A775ins_G776insYVMA, A775_G776insSVMA, A775_G776insVVMA, G776del insVC, G776del insLC, G776del insAV, G776del insAVGC, S310F, S310Y, p95, V842I, or P780_Y781insGSP.

In some embodiments, the cancer in the subject comprises an EGFR mutation. In some embodiments, the EGFR mutation comprises a substitution in exon 18, a deletion in exon 19, a substitution in exon 20, an insertion in exon 20, a mutation in the extracellular domain, or a substitution in exon 21. In some embodiments, the EGFR mutation is del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, D770_N771insSVD EGFR, H773insNPH EGFR, A767_dupASV EGFR, or 773insAH EGFR. In some embodiments, the EGFR mutation is del19/T790M EGFR or L858R/T790M EGFR.

In another aspect, the present disclosure provides a method of treating an inflammatory disease in a subject in need thereof, comprising administering to the subject a therapeutically effective amount of a compound of Formula I, or a pharmaceutically acceptable salt thereof.

In some embodiments, the inflammatory disease is selected from psoriasis, eczema, and atherosclerosis.

In some embodiments, the inflammatory disease in the subject comprises a HER2 mutation. In some embodiments, the HER2 mutation comprises an insertion in exon 20, an in-frame deletion and insertion in exon 20, a substitution in the extracellular domain, an extracellular truncation, or a substitution in exon 30. In some embodiments, the HER2 mutation is A775ins_G776insYVMA, A775_G776insSVMA, A775_G776insVVMA, G776del insVC, G776del insLC, G776del insAV, G776del insAVGC, S310F, S310Y, p95, V842I, or P780_Y781insGSP.

In some embodiments, the inflammatory disease in the subject comprises an EGFR mutation. In some embodiments, the EGFR mutation comprises a substitution in exon 18, a deletion in exon 19, a substitution in exon 20, an insertion in exon 20, a mutation in the extracellular domain, or a substitution in exon 21. In some embodiments, the EGFR mutation is del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, D770_N771insSVD EGFR, H773insNPH EGFR, A767_dupASV EGFR, or 773insAH EGFR. In some embodiments, the EGFR mutation is del19/T790M EGFR or L858R/T790M EGFR.

The present disclosure discloses a process of preparation of compounds of Formula I, or its stereoisomers, tautomers, pharmaceutically acceptable salts, polymorphs, solvates and hydrates thereof, and to pharmaceutical compositions containing them.

The compounds of the present invention are useful in the treatment, prevention or suppression of diseases and disorders mediated by epidermal growth factor receptor (EGFR).

These and other features, aspects, and advantages of the present disclosure will become better understood with reference to the following description. This statement is provided to introduce a selection of concepts in simplified form. This statement is not intended to identify key features or essential features of the subject matter, nor is it intended to be used to limit the scope of the subject matter.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

### Definitions

In the structural formulae given herein and throughout the present disclosure, the following terms have the indicated meaning, unless specifically stated otherwise.

The term “optionally substituted” as used herein means that the group in question is either unsubstituted or substituted with one or more of the substituents specified. In some embodiments, when the group in question is substituted with more than one substituent, the substituent is the same. In some embodiments, when the group in question is substituted with more than one substituent, the substituent is different.

The term “alkyl” refers to a monoradical branched or unbranched saturated hydrocarbon chain having 1, 2, 3, 4, 5, or 6 carbon atoms. This term is exemplified by groups such as methyl, ethyl, n-propyl, iso-propyl, n-butyl, iso-butyl, t-butyl, n-hexyl, and the like.

The term “cycloalkyl” refers to unless otherwise mentioned, carbocyclic groups of from 3 to 6 carbon atoms having a single cyclic ring or multiple condensed rings or spirocyclic rings or bridged rings. This definition encompasses rings that are saturated or partially unsaturated. Such cycloalkyl groups include, by way of example, single ring structures such as cyclopropyl, cyclobutyl, cyclopentyl, cyclopentenyl, cyclohexyl, cyclohexenyl, and the like.

“Halo” or “Halogen”, alone or in combination with any other term means halogens such as chloro (Cl), fluoro (F), bromo (Br) and iodo (I).

The term “aryl” refers to a radical derived from a hydrocarbon ring system comprising hydrogen, 6 to 30 carbon atoms and at least one aromatic ring. This definition encompasses monocyclic, bicyclic, tricyclic or tetracyclic ring system, as well as fused or bridged ring systems. Aryl radicals include, but are not limited to, aryl radicals derived from the hydrocarbon ring systems of aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

The term “phenyl” refers to an aromatic carbocyclic group of 6 carbon atoms having a single ring.

The term “phenyl alkyl” refers to a monoradical branched or unbranched saturated hydrocarbon chain having 1, 2, 3, 4, 5, or 6 carbon atoms substituted with an aromatic carbocyclic group of 6 carbon atoms having a single ring.

The term “heteroaryl” refers to an aromatic cyclic group having 5, or 6 carbon atoms and 1, 2, or 3 heteroatoms selected from oxygen, nitrogen and sulfur within at least one ring. An “X-linked heteroaryl” refers to a heteroaryl connected to the rest of the molecule via an X atom. For example,

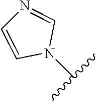 [SMILES: *n1ccnc1]

is an N-linked imidazolyl, while

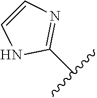 [SMILES: *c1ncc[nH]1]

is a C-linked imidazolyl.

The term “heterocycloalkyl” refers to a saturated, partially unsaturated, or unsaturated group having a single ring or multiple condensed rings or spirocyclic rings, or bridged rings unless otherwise mentioned, having from 2 to 10 carbon atoms and from 1 to 3 hetero atoms, selected from nitrogen, sulfur, phosphorus, and/or oxygen within the ring.

The term “alkenyl” refers to unsaturated aliphatic groups having at least one double bond.

The term “alkynyl” refers to unsaturated aliphatic groups having at least one triple bond.

The term “amino” refers to the —NH₂ radical.

The term “cyano” refers to the —CN radical.

The term “hydroxy” or “hydroxyl” refers to the —OH radical.

The term “heteroalkyl” refers to an alkyl radical as described above where one or more carbon atoms of the alkyl is replaced with an O, N or S atom. Unless stated otherwise specifically in the specification, the heteroalkyl group is optionally substituted as described below. Representative heteroalkyl groups include, but are not limited to —OCH₂CH₂OMe, —OCH₂CH₂OCH₂CH₂NH₂, and —OCH₂CH₂OCH₂CH₂OCH₂CH₂N(Me)₂.

The term “haloalkyl” refers to an alkyl radical as described above where one or more carbon atoms of the alkyl is replaced with a halogen atom. In some embodiments, the haloalkyl group is optionally substituted as described below. Representative haloalkyl groups include, but are not limited to, fluoromethyl, difluoromethyl, trifluoromethyl, difluoroethyl, and trifluoroethyl.

The term “aminoalkyl” refers to an alkyl group substituted with an amino (NH2) group.

The term “alkoxy” refers to the group R—O—, where R is optionally substituted alkyl or optionally substituted cycloalkyl, or optionally substituted alkenyl or optionally substituted alkynyl; or optionally substituted cycloalkenyl, where alkyl, alkenyl, alkynyl, cycloalkyl and cycloalkenyl are as defined herein. Representative examples of alkoxy groups include but are not limited to methoxy, ethoxy, n-propoxy, iso-propoxy, n-butoxy, tert-butoxy, sec-butoxy, n-pentoxy, n-hexoxy, 1,2-dimethylbutoxy, trifluoromethoxy, and the like.

In some embodiments, the compounds of the present disclosure have the ability to crystallize in more than one form, a characteristic known as polymorphism, and all such polymorphic forms (“polymorphs”) are encompassed within the scope of the disclosure. Polymorphism generally can occur as a response to changes in temperature or pressure or both, and can also result from variations in the crystallization process. Polymorphs can be distinguished by various physical characteristics, and typically the X-ray diffraction patterns, solubility behavior, and melting point of the compound are used to distinguish polymorphs.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine chlorine, iodine, phosphorus, such as, for example, ²H, ³H, ¹³C, ¹⁴C, ¹⁵N, ¹⁸O, ¹⁷O, ³⁵S, ¹⁸F, ³⁶Cl, ¹²³I, ¹²⁴I, ¹²⁵I, ¹³¹I, ³²P and ³³P. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements. In some embodiments, the compounds described herein can exist as isotopic variants. In some embodiments, an isotopic variant of a compound described herein has one or more hydrogen atoms replaced by deuterium.

In some embodiments, the compounds described herein contain one or more chiral centers and/or double bonds and therefore, exist as stereoisomers, such as double-bond isomers (i.e., geometric isomers), regioisomers, enantiomers or diastereomers. Accordingly, the chemical structures depicted herein encompass all possible enantiomers and stereoisomers of the illustrated or identified compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the person skilled in the art. In some embodiments, the compounds also exist in several tautomeric forms including the enol form, the keto form and mixtures thereof. Accordingly, the chemical structures depicted herein encompass all possible tautomeric forms of the illustrated or identified compounds.

In some embodiments, compounds exist in unsolvated forms as well as solvated forms, including hydrated forms and as N-oxides. In some embodiments, compounds are hydrated, solvated or N-oxides. In some embodiments, certain compounds exist in multiple crystalline or amorphous forms. Also contemplated within the scope of the disclosure are congeners, analogs, hydrolysis products, metabolites and precursor or prodrugs of the compound. In general, unless otherwise indicated, all physical forms are equivalent for the uses contemplated herein and are intended to be within the scope of the present disclosure.

“Pharmaceutically acceptable salt” embraces salts with a pharmaceutically acceptable acid or base. Pharmaceutically acceptable acids include both inorganic acids, for example hydrochloric, sulfuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methanesulfonic, ethanesulfonic, benzenesulfonic or p-toluenesulfonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines.

“Pharmaceutical composition” refers to one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition comprising a compound of the present disclosure and a pharmaceutically acceptable carrier.

“Carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water is a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient. The formulation should suit the mode of administration.

“Combined” or “in combination” or “combination” should be understood as a functional coadministration, encompassing scenarios wherein compounds are administered separately, in different formulations, different modes of administration (for example subcutaneous, intravenous or oral) and different times of administration. In some embodiments, the individual compounds of such combinations are administered sequentially in separate pharmaceutical compositions. In some embodiments, the individual compounds of such combinations are administered simultaneously in combined pharmaceutical compositions.

### Compounds

In one aspect, provided herein is a compound of Formula I:

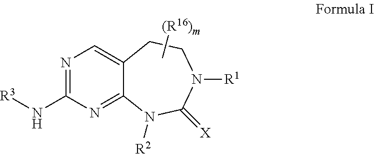

or a pharmaceutically acceptable salt thereof, wherein:
X is O or S;
R¹ is —(C(R⁴)₂)ₙR⁵, wherein R⁵ is substituted with 0, 1, or 2 R^{5′};
   n is 0, 1, 2, or 3;
   each R⁴ is independently hydrogen, alkyl, halo, haloalkyl, hydroxy, alkoxy, or heteroalkyl;
   R⁵ is cycloalkyl, heterocycloalkyl, aryl, heteroaryl;
   each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy;
      each R⁶ is independently alkyl, cycloalkyl, aryl, or heteroaryl;
R² is aryl, heteroaryl, cycloalkyl, or heterocycloalkyl, wherein the aryl, heteroaryl, cycloalkyl, or heterocycloalkyl is substituted with at least one R⁷ and 0, 1, or 2 R⁸;
   each R⁷ is independently

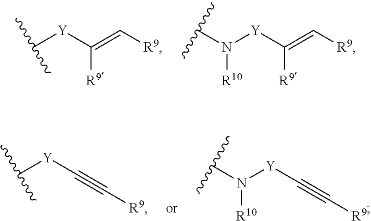

Y is —C(═O)—, —S(═O)—, or —S(═O)₂—;
R⁹ and R^{9′} are independently hydrogen, halo, alkyl, haloalkyl, cycloalkyl, heteroalkyl, or (alkyl)heterocycloalkyl;
R¹⁰ is hydrogen, alkyl, haloalkyl, or cycloalkyl;
each R⁸ is independently aryl, heteroaryl, alkyl, cycloalkyl, heterocycloalkyl, halo, heteroalkyl, haloalkyl, cyano, hydroxy, amino, —N(R¹¹)₂, —S(═O)₂alkyl, —S(═O)₂aryl, —S(═O)₂heteroaryl, or alkoxy;
   each R¹¹ is independently alkyl, cycloalkyl, aryl, or heteroaryl;
R³ is alkyl, aryl, heteroaryl, cycloalkyl, or heterocycloalkyl substituted with 0, 1, 2, or 3 R¹²;
   each R¹² is independently aryl, heteroaryl, alkyl, heteroalkyl, haloalkyl, halo, cyano, alkoxy, heterocycloalkyl, —N(R¹³)₂, —S(═O)₂NH₂, —S(═O)₂NMe₂, —S(═O)₂alkyl, —S(═O)₂aryl, —S(═O)₂heteroaryl, or cycloalkyl, wherein the aryl, heteroaryl, heterocycloalkyl, or cycloalkyl are each independently substituted with 0, 1, or 2 R¹⁴;
      each R¹³ is independently alkyl, cycloalkyl, aryl, or heteroaryl
      each R¹⁴ is independently aryl, heteroaryl, alkyl, cycloalkyl, heterocycloalkyl, halo, heteroalkyl, haloalkyl, cyano, hydroxy, amino, —N(R¹⁵)₂, —S(═O)₂alkyl, —S(═O)₂aryl, —S(═O)₂heteroaryl, or alkoxy;
         each R¹⁵ is independently alkyl, cycloalkyl, aryl, or heteroaryl;
each R¹⁶ is independently aryl, heteroaryl, alkyl, heteroalkyl, haloalkyl, halo, cyano, hydroxy, amino, alkoxy, heterocycloalkyl, —N(R¹⁷)₂, —S(═O)₂NH₂, —S(═O)₂NMe₂, —S(═O)₂alkyl, —S(═O)₂aryl, —S(═O)₂heteroaryl, or cycloalkyl, wherein the aryl, heteroaryl, heterocycloalkyl, or cycloalkyl are each independently substituted with 0, 1, or 2 R¹⁸;
   each R¹⁷ is independently alkyl, cycloalkyl, aryl, or heteroaryl
   each R¹⁸ is independently aryl, heteroaryl, alkyl, cycloalkyl, heterocycloalkyl, halo, heteroalkyl, haloalkyl, cyano, hydroxy, amino, —N(R¹⁹)₂, —S(═O)₂alkyl, —S(═O)₂aryl, —S(═O)₂heteroaryl, or alkoxy;
      each R¹⁹ is independently alkyl, cycloalkyl, aryl, or heteroaryl; and
m is 0, 1, 2, 3, or 4.

Some embodiments provided herein describe a compound of Formula I or a pharmaceutically acceptable salt thereof, wherein:
X is O;
R¹ is —(C(R⁴)₂)ₙR⁵, wherein R⁵ is substituted with 0 or 1 R^{5′};
   n is 1;
   each R⁴ is independently hydrogen or alkyl;
   R⁵ is aryl;
   each R^{5′} is halo;
R² is aryl substituted with one R⁷;
   R⁷ is

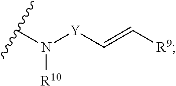

Y is —C(═O)—, or —S(═O)₂—;
R⁹ is hydrogen or heteroalkyl;
R¹⁰ is hydrogen or alkyl;
R³ is heteroaryl substituted with 1 or 2 R¹²; and
   each R¹² is independently alkyl or halo.

Some embodiments provided herein describe a compound of Formula I or a pharmaceutically acceptable salt thereof, wherein:
X is O;
R¹ is —(C(R⁴)₂)ₙR⁵, wherein R⁵ is substituted with 0 or 1 R^{5′};
   n is 0;
   R⁵ is aryl;
   each R^{5′} is halo;
R² is aryl substituted with one R⁷;
   R⁷ is

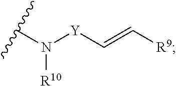

Y is —C(═O)—;
R⁹ is hydrogen or heteroalkyl;
R¹⁰ is hydrogen or alkyl;
R³ is heteroaryl substituted with 1 or 2 R¹²; and
   each R¹² is independently alkyl or halo.

Some embodiments provided herein describe a compound of Formula I or a pharmaceutically acceptable salt thereof, wherein:
X is O;
R¹ is —(C(R⁴)₂)ₙR⁵, wherein R⁵ is substituted with 0 or 1 R^{5′};
   n is 1;
   each R⁴ is hydrogen;
   R⁵ is aryl;
   each R^{5′} is halo;
R² is cycloalkyl substituted with one R⁷;
   R⁷ is

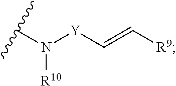

Y is —C(═O)—;
R⁹ is hydrogen or heteroalkyl;
R¹⁰ is hydrogen;
R³ is heteroaryl substituted with 1 or 2 R¹²; and
   each R¹² is independently alkyl or halo.

Some embodiments provided herein describe a compound of Formula I or a pharmaceutically acceptable salt thereof, wherein:
X is O;
R¹ is —(C(R⁴)₂)ₙR⁵, wherein R⁵ is substituted with 0 or 1 R^{5′};
   n is 0;
   R⁵ is aryl;
   each R^{5′} is halo;
R² is cycloalkyl substituted with one R⁷;
   R⁷ is

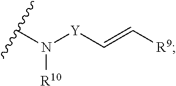

Y is —C(═O)—;
R⁹ is hydrogen;
R¹⁰ is hydrogen;
R³ is heteroaryl substituted with 1 or 2 R¹²; and
   each R¹² is independently alkyl or halo.

Some embodiments provided herein describe a compound of Formula I or a pharmaceutically acceptable salt thereof, wherein:
X is O;
R¹ is —(C(R⁴)₂)ₙR⁵, wherein R⁵ is substituted with 0 or 1 R^{5′};
   n is 1;
   each R⁴ is hydrogen;
   R⁵ is aryl;
   each R^{5′} is halo;
R² is heterocycloalkyl substituted with one R⁷;
   R⁷ is

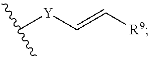

Y is —C(═O)—;
R⁹ is hydrogen or heteroalkyl;
R³ is heteroaryl substituted with 1 or 2 R¹²; and
   each R¹² is independently alkyl or halo.

Some embodiments provided herein describe a compound of Formula I or a pharmaceutically acceptable salt thereof, wherein:
X is O;
R¹ is —(C(R⁴)₂)ₙR⁵, wherein R⁵ is substituted with 0 or 1 R^{5′};
   n is 0;
   R⁵ is aryl;
   each R^{5′} is halo;
R² is heterocycloalkyl substituted with one R⁷;
   R⁷ is

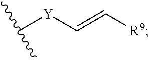

Y is —C(═O)—;
R⁹ is hydrogen;
R³ is heteroaryl substituted with 1 or 2 R¹²; and
   each R¹² is independently alkyl or halo.

Some embodiments provided herein describe a compound of Formula I-A

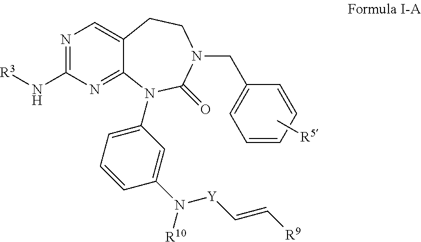

or a pharmaceutically acceptable salt thereof, wherein:
   each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy;
      each R⁶ is independently alkyl, cycloalkyl, aryl, or heteroaryl; Y is —C(═O)—, —S(═O)—, or —S(═O)₂—;
      R⁹ is hydrogen, halo, alkyl, cycloalkyl, or heteroalkyl;
      R¹⁰ is hydrogen or alkyl;
R³ is heteroaryl substituted with 0, 1, 2, or 3 R¹²;
   each R¹² is independently alkyl, heteroalkyl, haloalkyl, halo, cyano, alkoxy, heterocycloalkyl, —N(R¹³)₂, or cycloalkyl, wherein the heterocycloalkyl or cycloalkyl are each independently substituted with 0, 1, or 2 R¹⁴;
      each R¹³ is independently alkyl, cycloalkyl, aryl, or heteroaryl
      each R¹⁴ is independently aryl, heteroaryl, alkyl, cycloalkyl, heterocycloalkyl, halo, heteroalkyl, haloalkyl, cyano, hydroxy, amino, —N(R¹⁵)₂, —S(═O)₂alkyl, —S(═O)₂aryl, —S(═O)₂heteroaryl, or alkoxy; and each R¹⁵ is independently alkyl, cycloalkyl, aryl, or heteroaryl.

Some embodiments provided herein describe a compound of Formula I-B

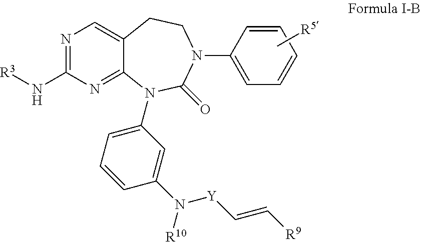

or a pharmaceutically acceptable salt thereof, wherein:
   each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy;
      each R⁶ is independently alkyl, cycloalkyl, aryl, or heteroaryl; Y is —C(═O)—, —S(═O)—, or —S(═O)₂—;
      R⁹ is hydrogen, halo, alkyl, cycloalkyl, or heteroalkyl;
      R¹⁰ is hydrogen or alkyl;
R³ is heteroaryl substituted with 0, 1, 2, or 3 R¹²;
   each R¹² is independently alkyl, heteroalkyl, haloalkyl, halo, cyano, alkoxy, heterocycloalkyl, —N(R¹³)₂, or cycloalkyl, wherein the heterocycloalkyl or cycloalkyl are each independently substituted with 0, 1, or 2 R¹⁴;
      each R¹³ is independently alkyl, cycloalkyl, aryl, or heteroaryl
      each R¹⁴ is independently aryl, heteroaryl, alkyl, cycloalkyl, heterocycloalkyl, halo, heteroalkyl, haloalkyl, cyano, hydroxy, amino, —N(R¹⁵)₂, —S(═O)₂alkyl, —S(═O)₂aryl, —S(═O)₂heteroaryl, or alkoxy; and
         each R¹⁵ is independently alkyl, cycloalkyl, aryl, or heteroaryl.

Some embodiments provided herein describe a compound of Formula I-C

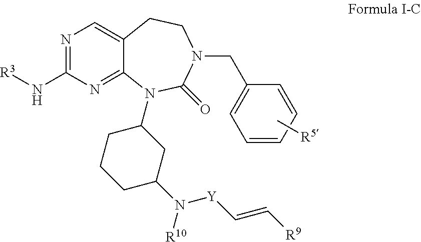

or a pharmaceutically acceptable salt thereof, wherein:
   each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy;
      each R⁶ is independently alkyl, cycloalkyl, aryl, or heteroaryl; Y is —C(═O)—, —S(═O)—, or —S(═O)₂—;
      R⁹ is hydrogen, halo, alkyl, cycloalkyl, or heteroalkyl;
      R¹⁰ is hydrogen or alkyl;
R³ is heteroaryl substituted with 0, 1, 2, or 3 R¹²;
   each R¹² is independently alkyl, heteroalkyl, haloalkyl, halo, cyano, alkoxy, heterocycloalkyl, —N(R¹³)₂, or cycloalkyl, wherein the heterocycloalkyl or cycloalkyl are each independently substituted with 0, 1, or 2 R¹⁴;
      each R¹³ is independently alkyl, cycloalkyl, aryl, or heteroaryl
      each R¹⁴ is independently aryl, heteroaryl, alkyl, cycloalkyl, heterocycloalkyl, halo, heteroalkyl, haloalkyl, cyano, hydroxy, amino, —N(R¹⁵)₂, —S(═O)₂alkyl, —S(═O)₂aryl, —S(═O)₂heteroaryl, or alkoxy; and
         each R¹⁵ is independently alkyl, cycloalkyl, aryl, or heteroaryl.

Some embodiments provided herein describe a compound of Formula I-D

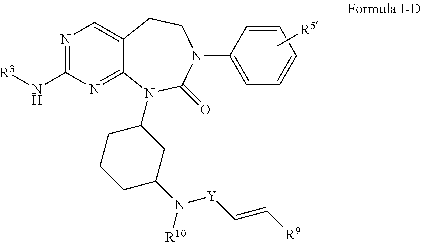

or a pharmaceutically acceptable salt thereof, wherein:
   each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy;
      each R⁶ is independently alkyl, cycloalkyl, aryl, or heteroaryl; Y is —C(═O)—, —S(═O)—, or —S(═O)₂—;
      R⁹ is hydrogen, halo, alkyl, cycloalkyl, or heteroalkyl;
      R¹⁰ is hydrogen or alkyl;
R³ is heteroaryl substituted with 0, 1, 2, or 3 R¹²;
   each R¹² is independently alkyl, heteroalkyl, haloalkyl, halo, cyano, alkoxy, heterocycloalkyl, —N(R¹³)₂, or cycloalkyl, wherein the heterocycloalkyl or cycloalkyl are each independently substituted with 0, 1, or 2 R¹⁴;
      each R¹³ is independently alkyl, cycloalkyl, aryl, or heteroaryl
      each R¹⁴ is independently aryl, heteroaryl, alkyl, cycloalkyl, heterocycloalkyl, halo, heteroalkyl, haloalkyl, cyano, hydroxy, amino, —N(R¹⁵)₂, —S(═O)₂alkyl, —S(═O)₂aryl, —S(═O)₂heteroaryl, or alkoxy; and
         each R¹⁵ is independently alkyl, cycloalkyl, aryl, or heteroaryl.

Some embodiments provided herein describe a compound of Formula I-E

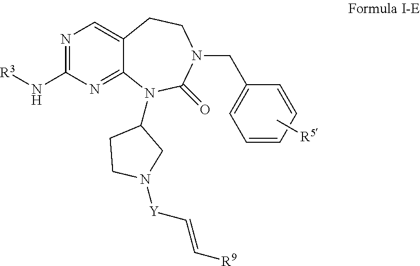

or a pharmaceutically acceptable salt thereof, wherein:
   each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy;
      each R⁶ is independently alkyl, cycloalkyl, aryl, or heteroaryl;
      Y is —C(═O)—, —S(═O)—, or —S(═O)₂—;
      R⁹ is hydrogen, halo, alkyl, cycloalkyl, or heteroalkyl;
R³ is heteroaryl substituted with 0, 1, 2, or 3 R¹²;
   each R¹² is independently alkyl, heteroalkyl, haloalkyl, halo, cyano, alkoxy, heterocycloalkyl, —N(R¹³)₂, or cycloalkyl, wherein the heterocycloalkyl or cycloalkyl are each independently substituted with 0, 1, or 2 R¹⁴;
      each R¹³ is independently alkyl, cycloalkyl, aryl, or heteroaryl
      each R¹⁴ is independently aryl, heteroaryl, alkyl, cycloalkyl, heterocycloalkyl, halo, heteroalkyl, haloalkyl, cyano, hydroxy, amino, —N(R¹⁵)₂, —S(═O)₂alkyl, —S(═O)₂aryl, —S(═O)₂heteroaryl, or alkoxy; and
         each R¹⁵ is independently alkyl, cycloalkyl, aryl, or heteroaryl.

Some embodiments provided herein describe a compound of Formula I-F

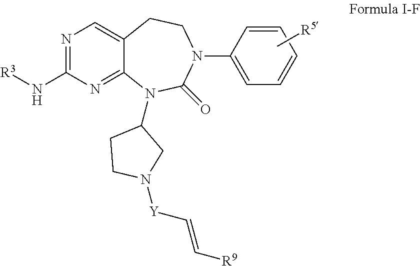

or a pharmaceutically acceptable salt thereof, wherein:
   each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy;
      each R⁶ is independently alkyl, cycloalkyl, aryl, or heteroaryl;
      Y is —C(═O)—, —S(═O)—, or —S(═O)₂—;
      R⁹ is hydrogen, halo, alkyl, cycloalkyl, or heteroalkyl;
R³ is heteroaryl substituted with 0, 1, 2, or 3 R¹²;
   each R¹² is independently alkyl, heteroalkyl, haloalkyl, halo, cyano, alkoxy, heterocycloalkyl, —N(R¹³)₂, or cycloalkyl, wherein the heterocycloalkyl or cycloalkyl are each independently substituted with 0, 1, or 2 R¹⁴;
      each R¹³ is independently alkyl, cycloalkyl, aryl, or heteroaryl
      each R¹⁴ is independently aryl, heteroaryl, alkyl, cycloalkyl, heterocycloalkyl, halo, heteroalkyl, haloalkyl, cyano, hydroxy, amino, —N(R¹⁵)₂, —S(═O)₂alkyl, —S(═O)₂aryl, —S(═O)₂heteroaryl, or alkoxy; and
         each R¹⁵ is independently alkyl, cycloalkyl, aryl, or heteroaryl.

Some embodiments provided herein describe a compound of Formula I-G

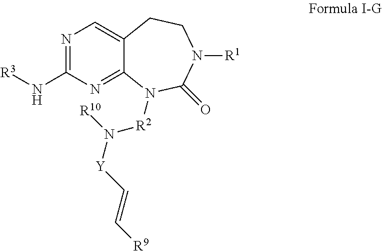

or a pharmaceutically acceptable salt thereof, wherein:
R¹ is —(C(R⁴)₂)ₙR⁵, wherein R⁵ is substituted with 0, 1, or 2 R^{5′};
   n is 0, 1, 2, or 3;
   each R⁴ is independently hydrogen, alkyl, halo, haloalkyl, hydroxy, alkoxy, or heteroalkyl;
   R⁵ is aryl or C-linked heteroaryl;
   each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy;
      each R⁶ is independently alkyl, cycloalkyl, aryl, or heteroaryl;
R² is aryl, heteroaryl, cycloalkyl, or heterocycloalkyl, wherein the aryl, cycloalkyl, or heterocycloalkyl is substituted with 0, 1, or 2 R⁸;
   Y is —C(═O)—, —S(═O)—, or —S(═O)₂—;
   R⁹ is hydrogen, halo, alkyl, cycloalkyl, or heteroalkyl;
   R¹⁰ is hydrogen, alkyl, haloalkyl, or cycloalkyl;
   each R⁸ is independently aryl, heteroaryl, alkyl, cycloalkyl, heterocycloalkyl, halo, heteroalkyl, haloalkyl, cyano, hydroxy, amino, —N(R¹¹)₂, —S(═O)₂alkyl, —S(═O)₂aryl, —S(═O)₂heteroaryl, or alkoxy;
   each R¹¹ is independently alkyl, cycloalkyl, aryl, or heteroaryl;
R³ is heteroaryl substituted with 0, 1, 2, or 3 R¹²;
   each R¹² is independently aryl, heteroaryl, alkyl, heteroalkyl, haloalkyl, halo, cyano, alkoxy, heterocycloalkyl, —N(R¹³)₂, —S(═O)₂NH₂, —S(═O)₂alkyl, —S(═O)₂aryl, —S(═O)₂heteroaryl, or cycloalkyl, wherein the aryl, heteroaryl, heterocycloalkyl, or cycloalkyl are each independently substituted with 0, 1, or 2 R¹⁴;
      each R¹³ is independently alkyl, cycloalkyl, aryl, or heteroaryl;
      each R¹⁴ is independently aryl, heteroaryl, alkyl, cycloalkyl, heterocycloalkyl, halo, heteroalkyl, haloalkyl, cyano, hydroxy, amino, —N(R¹⁵)₂, —S(═O)₂alkyl, —S(═O)₂aryl, —S(═O)₂heteroaryl, or alkoxy; and
each R¹⁵ is independently alkyl, cycloalkyl, aryl, or heteroaryl.

Some embodiments provided herein describe a compound of Formula I-H

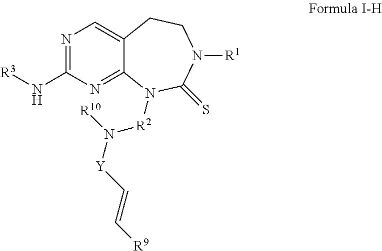

or a pharmaceutically acceptable salt thereof, wherein:
R¹ is —(C(R⁴)₂)ₙR⁵, wherein R⁵ is substituted with 0, 1, or 2 R^{5′};
   n is 0, 1, 2, or 3;
   each R⁴ is independently hydrogen, alkyl, halo, haloalkyl, hydroxy, alkoxy, or heteroalkyl;
   R⁵ is aryl or C-linked heteroaryl;
      each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy;
         each R⁶ is independently alkyl, cycloalkyl, aryl, or heteroaryl;
R² is aryl, heteroaryl, cycloalkyl, or heterocycloalkyl, wherein the aryl, cycloalkyl, or heterocycloalkyl is substituted with 0, 1, or 2 R⁸;
   Y is —C(═O)—, —S(═O)—, or —S(═O)₂—;
   R⁹ is hydrogen, halo, alkyl, cycloalkyl, or heteroalkyl;
   R¹⁰ is hydrogen, alkyl, haloalkyl, or cycloalkyl;
   each R⁸ is independently aryl, heteroaryl, alkyl, cycloalkyl, heterocycloalkyl, halo, heteroalkyl, haloalkyl, cyano, hydroxy, amino, —N(R¹¹)₂, —S(═O)₂alkyl, —S(═O)₂aryl, —S(═O)₂heteroaryl, or alkoxy;
   each R¹¹ is independently alkyl, cycloalkyl, aryl, or heteroaryl;
R³ is heteroaryl substituted with 0, 1, 2, or 3 R¹²;
   each R¹² is independently aryl, heteroaryl, alkyl, heteroalkyl, haloalkyl, halo, cyano, alkoxy, heterocycloalkyl, —N(R¹³)₂, —S(═O)₂NH₂, —S(═O)₂alkyl, —S(═O)₂aryl, —S(═O)₂heteroaryl, or cycloalkyl, wherein the aryl, heteroaryl, heterocycloalkyl, or cycloalkyl are each independently substituted with 0, 1, or 2 R¹⁴;
      each R¹³ is independently alkyl, cycloalkyl, aryl, or heteroaryl;
      each R¹⁴ is independently aryl, heteroaryl, alkyl, cycloalkyl, heterocycloalkyl, halo, heteroalkyl, haloalkyl, cyano, hydroxy, amino, —N(R¹⁵)₂, —S(═O)₂alkyl, —S(═O)₂aryl, —S(═O)₂heteroaryl, or alkoxy; and
each R¹⁵ is independently alkyl, cycloalkyl, aryl, or heteroaryl.

For any and all of the embodiments, substituents are selected from among a subset of the listed alternatives. For example, in some embodiments, n is 0, 1, 2, or 3. In some embodiments, n is 0, 1, or 2. In some embodiments, n is 0, 1, or 3. In some embodiments, n is 0, 2, or 3. In some embodiments, n is 1, 2, or 3. In some embodiments, n is 0 or 1. In some embodiments, n is 1 or 2. In some embodiments, n is 2 or 3. In some embodiments, n is 0 or 2. In some embodiments, n is 0 or 3. In some embodiments, n is 1 or 3. In some embodiments, n is 0. In some embodiments, n is 1. In some embodiments, n is 2. In some embodiments, n is 3.

In some embodiments, R⁵ is cyclopropyl, phenyl, naphthyl, anthracenyl, phenanthrenyl, chrysenyl, pyrenyl, pyridyl, pyrimidinyl, pyrazolyl, or imidazolyl. In some embodiments, R⁵ is phenyl, naphthyl, anthracenyl, phenanthrenyl, pyridyl, pyrimidinyl, pyrazolyl, or imidazolyl. In some embodiments, R⁵ is cyclopropyl. In some embodiments, R⁵ is phenyl. In some embodiments, R⁵ is naphthyl. In some embodiments, R⁵ is anthracenyl. In some embodiments, R⁵ is phenanthrenyl. In some embodiments, R⁵ is chrysenyl. In some embodiments, R⁵ is pyrenyl. In some embodiments, R⁵ is pyridyl. In some embodiments, R⁵ is pyrimidinyl. In some embodiments, R⁵ is pyrazolyl. In some embodiments, R⁵ is imidazolyl.

In some embodiments, R⁵ is unsubstituted. In some embodiments, R⁵ is substituted with 0, 1, or 2 R^{5′}. In some embodiments, R⁵ is substituted with 0 or 1 R^{5′}. In some embodiments, R⁵ is substituted with 0 or 2 R^{5′}. In some embodiments, R⁵ is substituted with 1 or 2 R^{5′}. In some embodiments, R⁵ is substituted with 1 R^{5′}. In some embodiments, R⁵ is substituted with 2 R^{5′}.

In some embodiments, each R⁴ is independently hydrogen, alkyl, halo, haloalkyl, hydroxy, alkoxy, or heteroalkyl. In some embodiments, each R⁴ is independently hydrogen, alkyl, halo, haloalkyl, or alkoxy. In some embodiments, each R⁴ is independently hydrogen, methyl, ethyl, n-propyl, iso-propyl, n-butyl, iso-butyl, sec-butyl, tert-butyl, fluoro, chloro, trifluoromethyl, trifluoroethyl, pentafluoroethyl, methoxy, ethoxy, or trifluoromethoxy. In some embodiments, each R⁴ is independently hydrogen, methyl, fluoro, trifluoromethyl, methoxy, or trifluoromethoxy. In some embodiments, each R⁴ is hydrogen. In some embodiments, each R⁴ is independently alkyl. In some embodiments, each R⁴ is independently halo. In some embodiments, each R⁴ is independently haloalkyl. In some embodiments, each R⁴ is hydroxy. In some embodiments, each R⁴ is independently alkoxy. In some embodiments, each R⁴ is independently heteroalkyl. In some embodiments, each R⁴ is methyl. In some embodiments, each R⁴ is ethyl. In some embodiments, each R⁴ is n-propyl. In some embodiments, each R⁴ is iso-propyl. In some embodiments, each R⁴ is n-butyl. In some embodiments, each R⁴ is iso-butyl. In some embodiments, each R⁴ is sec-butyl. In some embodiments, each R⁴ is tert-butyl. In some embodiments, each R⁴ is fluoro. In some embodiments, each R⁴ is chloro. In some embodiments, each R⁴ is trifluoromethyl. In some embodiments, each R⁴ is trifluoroethyl. In some embodiments, each R⁴ is pentafluoroethyl. In some embodiments, each R⁴ is methoxy. In some embodiments, each R⁴ is ethoxy. In some embodiments, each R⁴ is trifluoromethoxy.

In some embodiments, each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy. In some embodiments, each R^{5′} is independently aryl, heteroaryl, alkyl, heterocycloalkyl, halo, cyano, hydroxy, —N(R⁶)₂, or alkoxy. In some embodiments, each R^{5′} is independently aryl. In some embodiments, each R^{5′} is independently heteroaryl. In some embodiments, each R^{5′} is independently alkyl. In some embodiments, each R^{5′} is independently cycloalkyl. In some embodiments, each R^{5′} is independently heterocycloalkyl. In some embodiments, each R^{5′} is independently halo. In some embodiments, each R^{5′} is independently heteroalkyl. In some embodiments, each R^{5′} is independently haloalkyl. In some embodiments, each R^{5′} is cyano. In some embodiments, each R^{5′} is hydroxy. In some embodiments, each R^{5′} is amino. In some embodiments, each R^{5′} is independently —N(R⁶)₂. In some embodiments, each R^{5′} is independently —S(═O)₂alkyl. In some embodiments, each R^{5′} is independently —S(═O)₂aryl. In some embodiments, each R^{5′} is independently-S(═O)₂heteroaryl. In some embodiments, each R^{5′} is independently alkoxy. In some embodiments, each R^{5′} is independently phenyl, naphthyl, anthracenyl, phenanthrenyl, chrysenyl, pyrenyl, pyrrolyl, imidazolyl, pyrazolyl, triazolyl, tetrazolyl, indolyl, indazolyl, benzimidazolyl, azaindolyl, thiazolyl, isothiazolyl, oxazolyl, isoxazolyl, pyridinyl, pyrimidinyl, pyridazinyl, pyrazinyl, triazinyl, quinolinyl, isoquinolinyl, quinoxalinyl, quinazolinyl, cinnolinyl, naphthyridinyl, methyl, ethyl, n-propyl, iso-propyl, n-butyl, iso-butyl, sec-butyl, tert-butyl, azetidinyl, oxetanyl, pyrrolidinyl, imidazolidinyl, tetrahydrofuranyl, piperidinyl, piperazinyl, tetrahydropyranyl, morpholinyl, fluoro, chloro, cyano, hydroxy, —N(R⁶)₂, methoxy, ethoxy, or trifluoromethoxy. In some embodiments, each R^{5′} is independently phenyl, pyrrolyl, imidazolyl, thiazolyl, isothiazolyl, oxazolyl, isoxazolyl, pyridinyl, pyrimidinyl, methyl, ethyl, tert-butyl, pyrrolidinyl, piperidinyl, piperazinyl, morpholinyl, fluoro, chloro, cyano, hydroxy, —N(R⁶)₂, methoxy, ethoxy, or trifluoromethoxy. In some embodiments, each R^{5′} is independently phenyl, imidazolyl, pyridinyl, methyl, tert-butyl, pyrrolidinyl, morpholinyl, fluoro, cyano, hydroxy, —N(R⁶)₂, or methoxy. In some embodiments, each R^{5′} is phenyl. In some embodiments, each R^{5′} is naphthyl. In some embodiments, each R^{5′} is anthracenyl. In some embodiments, each R^{5′} is phenanthrenyl. In some embodiments, each R^{5′} is chrysenyl. In some embodiments, each R^{5′} is pyrenyl. In some embodiments, each R^{5′} is pyrrolyl. In some embodiments, each R^{5′} is imidazolyl. In some embodiments, each R^{5′} is pyrazolyl. In some embodiments, each R^{5′} is triazolyl. In some embodiments, each R^{5′} is tetrazolyl. In some embodiments, each R^{5′} is indolyl. In some embodiments, each R^{5′} is indazolyl. In some embodiments, each R^{5′} is benzimidazolyl. In some embodiments, each R^{5′} is azaindolyl. In some embodiments, each R^{5′} is thiazolyl. In some embodiments, each R^{5′} is isothiazolyl. In some embodiments, each R^{5′} is oxazolyl. In some embodiments, each R^{5′} is isoxazolyl. In some embodiments, each R^{5′} is pyridinyl. In some embodiments, each R^{5′} is pyrimidinyl. In some embodiments, each R^{5′} is pyridazinyl. In some embodiments, each R^{5′} is pyrazinyl. In some embodiments, each R^{5′} is triazinyl. In some embodiments, each R^{5′} is quinolinyl. In some embodiments, each R^{5′} is isoquinolinyl. In some embodiments, each R^{5′} is quinoxalinyl. In some embodiments, each R^{5′} is quinazolinyl. In some embodiments, each R^{5′} is cinnolinyl. In some embodiments, each R^{5′} is naphthyridinyl. In some embodiments, each R^{5′} is methyl. In some embodiments, each R^{5′} is ethyl. In some embodiments, each R^{5′} is n-propyl. In some embodiments, each R^{5′} is iso-propyl. In some embodiments, each R^{5′} is n-butyl. In some embodiments, each R^{5′} is iso-butyl. In some embodiments, each R^{5′} is sec-butyl. In some embodiments, each R^{5′} is tert-butyl. In some embodiments, each R^{5′} is azetidinyl. In some embodiments, each R^{5′} is oxetanyl. In some embodiments, each R^{5′} is pyrrolidinyl. In some embodiments, each R^{5′} is imidazolidinyl. In some embodiments, each R^{5′} is tetrahydrofuranyl. In some embodiments, each R^{5′} is piperidinyl. In some embodiments, each R^{5′} is piperazinyl. In some embodiments, each R^{5′} is tetrahydropyranyl. In some embodiments, each R^{5′} is morpholinyl. In some embodiments, each R^{5′} is fluoro. In some embodiments, each R^{5′} is chloro. In some embodiments, each R^{5′} is methoxy. In some embodiments, each R^{5′} is ethoxy. In some embodiments, each R^{5′} is trifluoromethoxy.

In some embodiments, each R⁶ is independently alkyl, cycloalkyl, aryl, or heteroaryl. In some embodiments, each R⁶ is independently alkyl or aryl. In some embodiments, each R⁶ is independently methyl, ethyl, n-propyl, iso-propyl, n-butyl, iso-butyl, sec-butyl, tert-butyl, phenyl, naphthyl, anthracenyl, phenanthrenyl, chrysenyl, or pyrenyl. In some embodiments, each R⁶ is independently methyl, ethyl, iso-propyl, tert-butyl, phenyl, or naphthyl. In some embodiments, each R⁶ is independently methyl or phenyl. In some embodiments, each R⁶ is methyl. In some embodiments, each R⁶ is ethyl. In some embodiments, each R⁶ is n-propyl. In some embodiments, each R⁶ is iso-propyl. In some embodiments, each R⁶ is n-butyl. In some embodiments, each R⁶ is iso-butyl. In some embodiments, each R⁶ is sec-butyl. In some embodiments, each R⁶ is tert-butyl. In some embodiments, each R⁶ is phenyl. In some embodiments, each R⁶ is naphthyl. In some embodiments, each R⁶ is anthracenyl. In some embodiments, each R⁶ is phenanthrenyl. In some embodiments, each R⁶ is chrysenyl. In some embodiments, each R⁶ is pyrenyl.

In some embodiments, X is S. In some embodiments, X is O.

In some embodiments, R² is aryl, heteroaryl, cycloalkyl, or heterocycloalkyl. In some embodiments, R² is aryl. In some embodiments, R² is heteroaryl. In some embodiments, R² is cycloalkyl. In some embodiments, R² is heterocycloalkyl. In some embodiments, R² is monocyclic. In some embodiments, R² is phenyl, cyclopropyl, cyclobutyl, cyclopentyl, cyclohexyl, pyrrolyl, imidazolyl, pyrazolyl, triazolyl, tetrazolyl, thiazolyl, isothiazolyl, oxazolyl, isoxazolyl, pyridinyl, pyrimidinyl, pyridazinyl, pyrazinyl, or triazinyl. In some embodiments, R² is phenyl, cyclohexyl, or pyrrolyl. In some embodiments, R² is phenyl. In some embodiments, R² is cyclopropyl. In some embodiments, R² is cyclobutyl. In some embodiments, R² is cyclopentyl. In some embodiments, R² is cyclohexyl. In some embodiments, R² is pyrrolyl. In some embodiments, R² is imidazolyl. In some embodiments, R² is pyrazolyl. In some embodiments, R² is triazolyl. In some embodiments, R² is tetrazolyl. In some embodiments, R² is thiazolyl. In some embodiments, R² is isothiazolyl. In some embodiments, R² is oxazolyl. In some embodiments, R² is isoxazolyl. In some embodiments, R² is pyridinyl. In some embodiments, R² is pyrimidinyl. In some embodiments, R² is pyridazinyl. In some embodiments, R² is pyrazinyl. In some embodiments, R² is triazinyl.

In some embodiments, R⁷ is

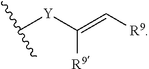

In some embodiments, R⁷ is

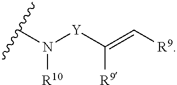

In some embodiments, R⁷ is

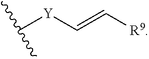

In some embodiments, R⁷ is

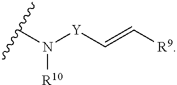

In some embodiments, R⁷ is

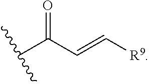

In some embodiments, R⁷ is

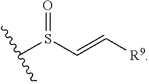

In some embodiments, R⁷ is

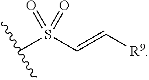

In some embodiments, R⁷ is

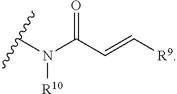

In some embodiments, R⁷ is

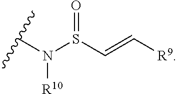

In some embodiments, R⁷ is

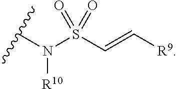

In some embodiments, R⁷ is

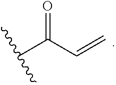 [SMILES: *C(=O)C=C]

In some embodiments, R⁷ is

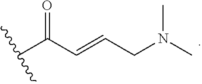 [SMILES: *C(=O)/C=C/CN(C)C]

In some embodiments, R⁷ is

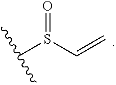 [SMILES: *S(=O)C=C]

In some embodiments, R⁷ is

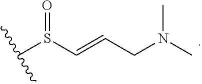 [SMILES: *S(=O)/C=C/CN(C)C]

In some embodiments, R⁷ is

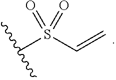 [SMILES: *S(=O)(=O)C=C]

In some embodiments, R⁷ is

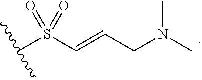 [SMILES: *S(=O)(=O)/C=C/CN(C)C]

In some embodiments, R⁷ is

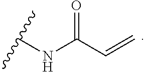 [SMILES: *NC(=O)C=C]

In some embodiments, R⁷ is

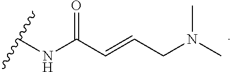 [SMILES: *NC(=O)/C=C/CN(C)C]

In some embodiments, R⁷ is

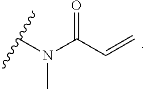 [SMILES: *N(C)C(=O)C=C]

In some embodiments, R⁷ is

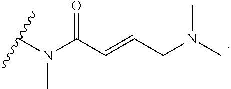 [SMILES: *N(C)C(=O)/C=C/CN(C)C]

In some embodiments, R⁷ is

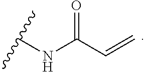 [SMILES: *NC(=O)C=C]

In some embodiments, R⁷ is

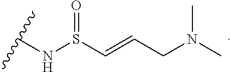 [SMILES: *NS(=O)/C=C/CN(C)C]

In some embodiments, R⁷ is

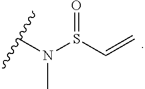 [SMILES: *N(C)S(=O)C=C]

In some embodiments, R⁷ is

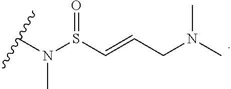 [SMILES: *N(C)S(=O)/C=C/CN(C)C]

In some embodiments, R⁷ is

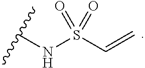 [SMILES: *NS(=O)(=O)C=C]

In some embodiments, R⁷ is

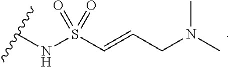 [SMILES: *NS(=O)(=O)/C=C/CN(C)C]

In some embodiments, R⁷ is

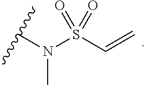 [SMILES: *N(C)S(=O)(=O)C=C]

In some embodiments, R⁷ is

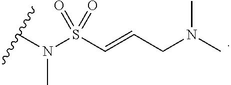 [SMILES: *N(C)S(=O)(=O)/C=C/CN(C)C]

In some embodiments, Y is —C(═O)—. In some embodiments, Y is —S(═O)—. In some embodiments, Y is —S(═O)₂—.

In some embodiments, R⁹ and R^{9′} are independently hydrogen, halo, alkyl, heteroalkyl, haloalkyl, or (alkyl)heterocycloalkyl. In some embodiments, R⁹ is hydrogen, halo, alkyl, cycloalkyl, or heteroalkyl. In some embodiments, R⁹ is hydrogen, halo, or heteroalkyl. In some embodiments, R⁹ and R^{9′} are independently hydrogen, fluoro, chloro, methyl, hydroxyethyl, methoxyethyl, methoxymethyl, hydroxymethyl, dimethylaminomethyl, 1-piperidinylmethyl, 1-morpholinylmethyl, or fluoromethyl. In some embodiments, R⁹ and R^{9′} are independently hydrogen, fluoro, chloro, hydroxyethyl, or methoxymethyl. In some embodiments, R⁹ is hydrogen, fluoro, chloro, hydroxyethyl, or methoxyethyl. In some embodiments, R⁹ is hydrogen. In some embodiments, R⁹ is fluoro. In some embodiments, R⁹ is chloro. In some embodiments, R⁹ is hydroxyethyl. In some embodiments, R⁹ is methoxyethyl. In some embodiments, R⁹ is methyl. In some embodiments, R⁹ is methoxymethyl. In some embodiments, R⁹ is dimethylaminomethyl. In some embodiments, R⁹ is 1-piperidinylmethyl. In some embodiments, R⁹ is 1-morpholinomethyl. In some embodiments, R⁹ is fluoromethyl. In some embodiments, R^{9′} is hydrogen. In some embodiments, R^{9′} is fluoro. In some embodiments, R^{9′} is chloro. In some embodiments, R^{9′} is hydroxyethyl. In some embodiments, R^{9′} is methoxyethyl. In some embodiments, R^{9′} is methyl. In some embodiments, R^{9′} is methoxymethyl. In some embodiments, R^{9′} is dimethylaminomethyl. In some embodiments, R^{9′} is 1-piperidinylmethyl. In some embodiments, R^{9′} is 1-morpholinomethyl. In some embodiments, R^{9′} is fluoromethyl.

In some embodiments, R¹⁰ is hydrogen, alkyl, haloalkyl, or cycloalkyl. In some embodiments, R¹⁰ is hydrogen, methyl, ethyl n-propyl, iso-propyl, n-butyl, sec-butyl, tert-butyl, trifluoromethyl, or cyclopropyl. In some embodiments, R¹⁰ is hydrogen or alkyl. In some embodiments, R¹⁰ is hydrogen, methyl, ethyl, n-propyl, iso-propyl, n-butyl, iso-butyl, sec-butyl, or tert-butyl. In some embodiments, R¹⁰ is hydrogen or methyl. In some embodiments, R¹⁰ is hydrogen. In some embodiments, R¹⁰ is methyl. In some embodiments, R¹⁰ is ethyl. In some embodiments, R¹⁰ is n-propyl. In some embodiments, R¹⁰ is iso-propyl. In some embodiments, R¹⁰ is n-butyl. In some embodiments, R¹⁰ is iso-butyl. In some embodiments, R¹⁰ is sec-butyl. In some embodiments, R¹⁰ is tert-butyl.

In some embodiments, R² is unsubstituted. In some embodiments, R² is substituted with 1 or 2 R⁸. In some embodiments, R² is substituted with 1 R⁸. In some embodiments, R² is substituted with 2 R⁸.

In some embodiments, each R⁸ is independently methyl, ethyl, n-propyl, iso-propyl, n-butyl, iso-butyl, sec-butyl, tert-butyl, fluoro, chloro, heteroalkyl, cyano, hydroxy, amino, —N(R¹¹)₂, methoxy, ethoxy, or trifluoromethoxy. In some embodiments, each R⁸ is independently methyl, ethyl, iso-propyl, tert-butyl, fluoro, chloro, —N(R¹¹)₂, hydroxyethyl, methoxyethyl, or cyano. In some embodiments, each R⁸ is methyl. In some embodiments, each R⁸ is ethyl. In some embodiments, each R⁸ is n-propyl. In some embodiments, each R⁸ is iso-propyl. In some embodiments, each R⁸ is n-butyl. In some embodiments, each R⁸ is iso-butyl. In some embodiments, each R⁸ is sec-butyl. In some embodiments, each R⁸ is tert-butyl. In some embodiments, each R⁸ is fluoro. In some embodiments, each R⁸ is chloro. In some embodiments, each R⁸ is independently —N(R¹¹)₂. In some embodiments, each R⁸ is hydroxyethyl. In some embodiments, each R⁸ is methoxyethyl. In some embodiments, each R⁸ is cyano.

In some embodiments, each R¹¹ is independently alkyl, cycloalkyl, aryl, or heteroaryl. In some embodiments, each R¹¹ is independently alkyl or aryl. In some embodiments, each R¹¹ is independently alkyl. In some embodiments, each R¹¹ is independently cycloalkyl. In some embodiments, each R¹¹ is independently aryl. In some embodiments, each R¹¹ is independently heteroaryl. In some embodiments, each R¹¹ is independently methyl, ethyl, n-propyl, iso-propyl, n-butyl, iso-butyl, sec-butyl, tert-butyl, phenyl, naphthyl, anthracenyl, phenanthrenyl, chrysenyl, or pyrenyl. In some embodiments, each R¹¹ is independently methyl, ethyl, n-propyl, iso-propyl, n-butyl, iso-butyl, sec-butyl, tert-butyl, phenyl, naphthyl, anthracenyl, or phenanthrenyl. In some embodiments, each R¹¹ is independently methyl, ethyl, iso-propyl, tert-butyl, phenyl, or naphthyl. In some embodiments, each R¹¹ is independently methyl or phenyl. In some embodiments, each R¹¹ is methyl. In some embodiments, each R¹¹ is ethyl. In some embodiments, each R¹¹ is n-propyl. In some embodiments, each R¹¹ is iso-propyl. In some embodiments, each R¹¹ is n-butyl. In some embodiments, each R¹¹ is iso-butyl. In some embodiments, each R¹¹ is sec-butyl. In some embodiments, each R¹¹ is tert-butyl. In some embodiments, each R¹¹ is phenyl. In some embodiments, each R¹¹ is naphthyl. In some embodiments, each R¹¹ is anthracenyl. In some embodiments, each R¹¹ is phenanthrenyl. In some embodiments, each R¹¹ is chrysenyl. In some embodiments, each R¹¹ is pyrenyl.

In some embodiments, R³ is phenyl, cyclopentyl, tetrahydropyranyl, oxetanyl, pyrrolyl, imidazolyl, pyrazolyl, triazolyl, tetrazolyl, indolyl, indazolyl, benzimidazolyl, azaindolyl, thiazolyl, isothiazolyl, oxazolyl, isoxazolyl, pyridinyl, pyrimidinyl, pyridazinyl, pyrazinyl, triazinyl, quinolinyl, isoquinolinyl, quinoxalinyl, quinazolinyl, cinnolinyl, or naphthyridinyl. In some embodiments, R³ is pyrrolyl, imidazolyl, pyrazolyl, triazolyl, tetrazolyl, indolyl, indazolyl, benzimidazolyl, azaindolyl, thiazolyl, isothiazolyl, oxazolyl, isoxazolyl, pyridinyl, pyrimidinyl, pyridazinyl, pyrazinyl, triazinyl, quinolinyl, isoquinolinyl, quinoxalinyl, quinazolinyl, cinnolinyl, or naphthyridinyl. In some embodiments, R³ is pyrazolyl, triazolyl, indolyl, indazolyl, thiazolyl, isothiazolyl, or pyridinyl. In some embodiments, R³ is phenyl, cyclopentyl, tetrahydropyranyl, oxetanyl, imidazolyl, triazolyl, indolyl, indazolyl, thiazolyl, isothiazolyl, or pyridinyl. In some embodiments, R³ is phenyl. In some embodiments, R³ is cyclopentyl. In some embodiments, R³ is tetrahydropyranyl. In some embodiments, R³ is oxetanyl. In some embodiments, R³ is pyrrolyl. In some embodiments, R³ is imidazolyl. In some embodiments, R³ is pyrazolyl. In some embodiments, R³ is triazolyl. In some embodiments, R³ is tetrazolyl. In some embodiments, R³ is indolyl. In some embodiments, R³ is indazolyl. In some embodiments, R³ is benzimidazolyl. In some embodiments, R³ is azaindolyl. In some embodiments, R³ is thiazolyl. In some embodiments, R³ is isothiazolyl. In some embodiments, R³ is oxazolyl. In some embodiments, R³ is isoxazolyl. In some embodiments, R³ is pyridinyl. In some embodiments, R³ is pyrimidinyl. In some embodiments, R³ is pyridazinyl. In some embodiments, R³ is pyrazinyl. In some embodiments, R³ is triazinyl. In some embodiments, R³ is quinolinyl. In some embodiments, R³ is isoquinolinyl. In some embodiments, R³ is quinoxalinyl. In some embodiments, R³ is quinazolinyl. In some embodiments, R³ is cinnolinyl. In some embodiments, R³ is naphthyridinyl.

In some embodiments, R³ is unsubstituted. In some embodiments, R³ is substituted with at least 1 R¹². In some embodiments, R³ is substituted with at least 2 R¹². In some embodiments, R³ is substituted with 1 R¹². In some embodiments, R³ is substituted with 2 R¹². In some embodiments, R³ is substituted with 3 R¹².

In some embodiments, R³ is

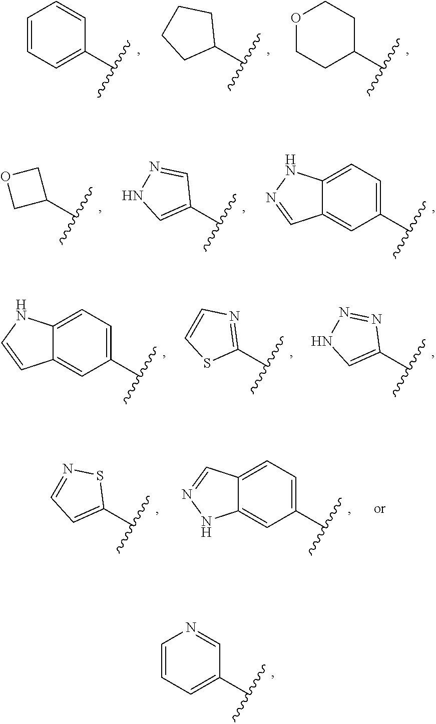

wherein R³ is substituted with 0 to 3 R¹². In some embodiments, R³ is

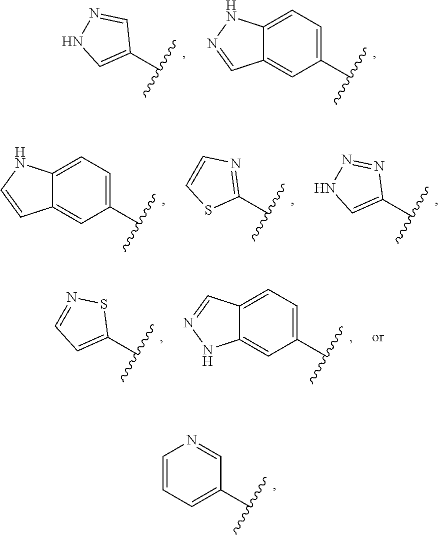

wherein R³ is substituted with 0 to 3 R¹². In some embodiments, R³ is

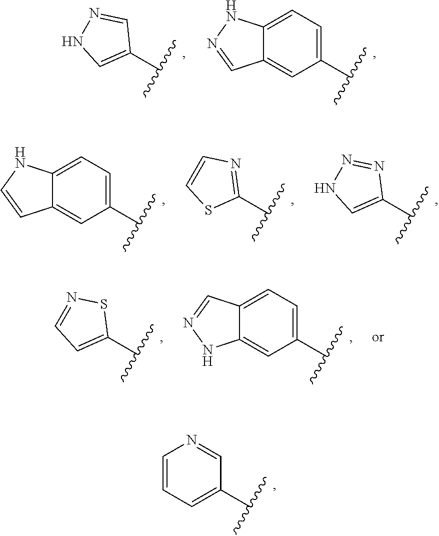

wherein R³ is substituted with 1 or 2 R¹².

In some embodiments, R³ is selected from:

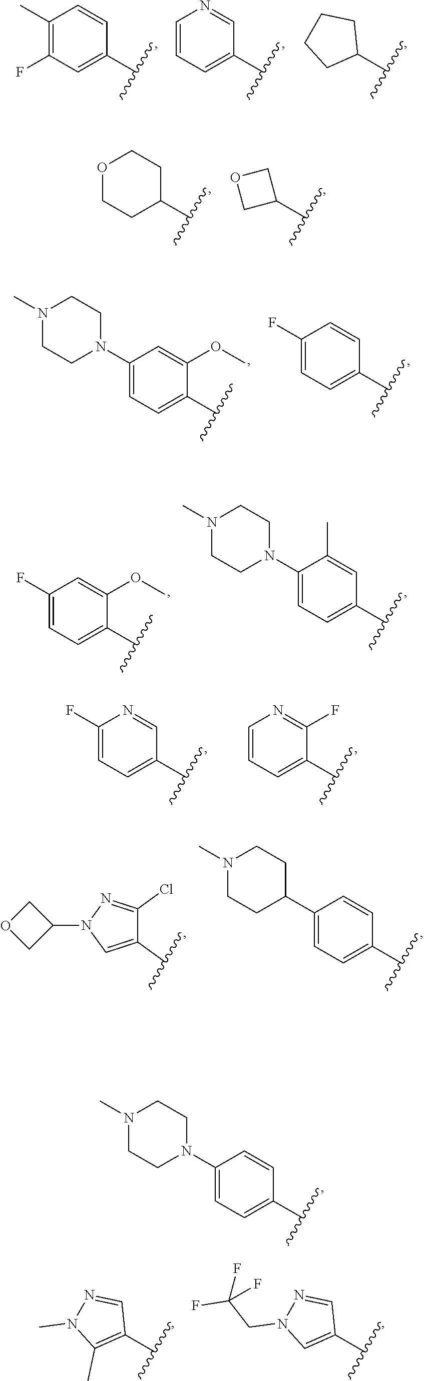

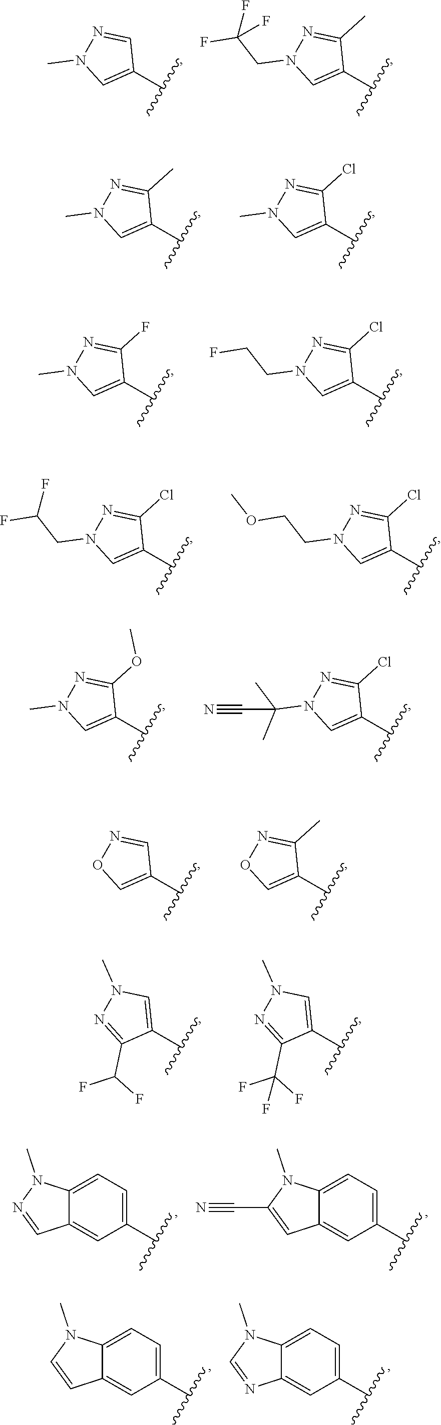

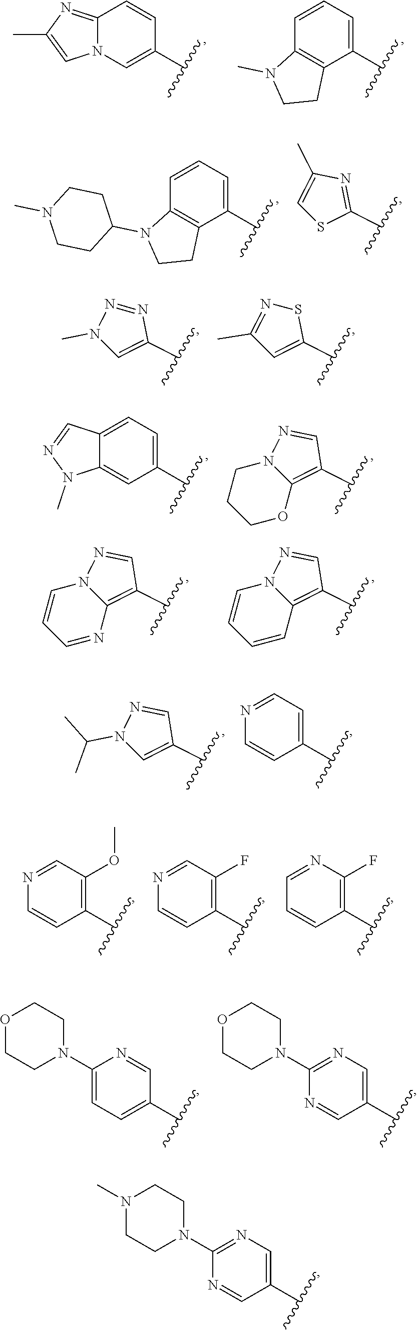

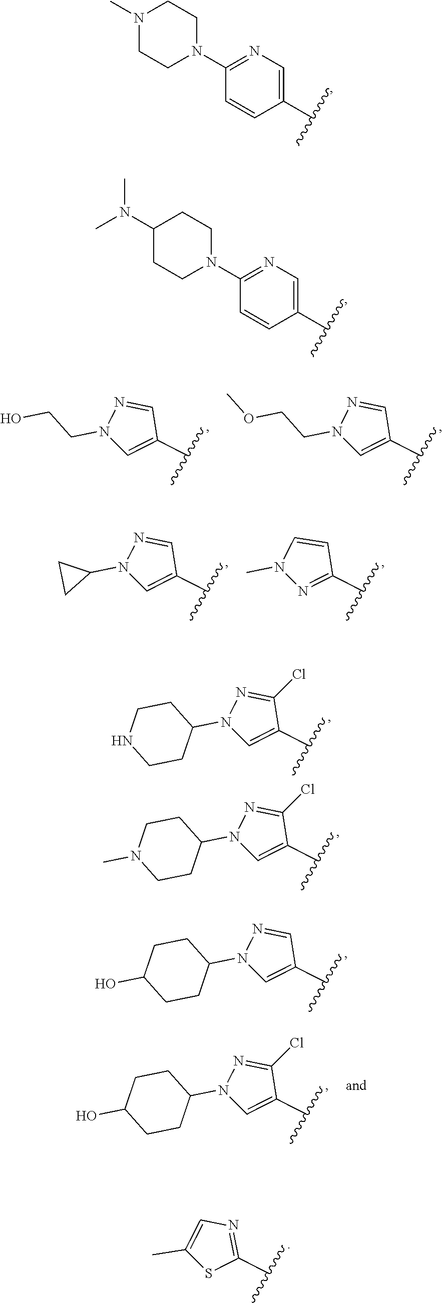

In some embodiments, R³ is selected from:

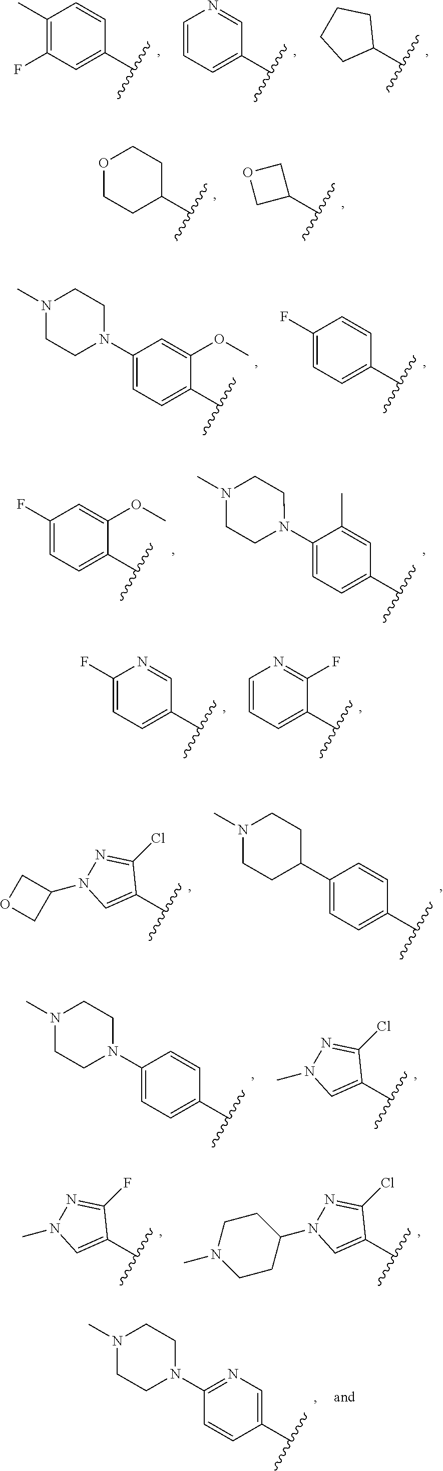

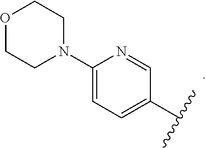 [SMILES: *c1ccc(N2CCOCC2)nc1]

In some embodiments, R³ is

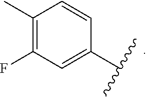 [SMILES: *c1ccc(C)c(F)c1]

In some embodiments, R³ is

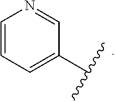 [SMILES: *c1cccnc1]

In some embodiments, R³ is

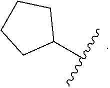 [SMILES: *C1CCCC1]

In some embodiments, R³ is

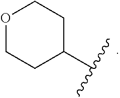 [SMILES: *C1CCOCC1]

In some embodiments, R³ is

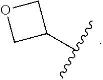 [SMILES: *C1COC1]

In some embodiments, R³ is

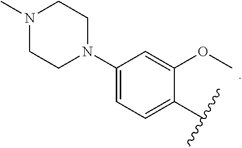

In some embodiments, R³ is

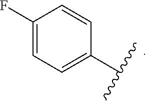 [SMILES: *c1ccc(F)cc1]

In some embodiments, R³ is

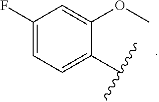 [SMILES: *c1ccc(F)cc1OC]

In some embodiments, R³ is

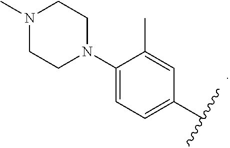 [SMILES: *c1ccc(N2CCN(C)CC2)c(C)c1]

In some embodiments, R³ is

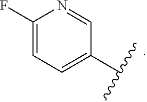 [SMILES: *c1ccc(F)nc1]

In some embodiments, R³ is

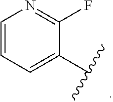 [SMILES: *c1cccnc1F]

In some embodiments, R³ is

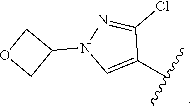 [SMILES: *c1cn(C2COC2)nc1Cl]

In some embodiments, R³ is

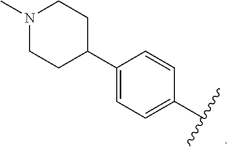 [SMILES: *c1ccc(C2CCN(C)CC2)cc1]

In some embodiments, R³ is

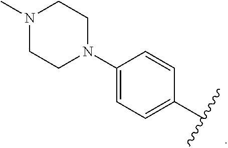 [SMILES: *c1ccc(N2CCN(C)CC2)cc1]

In some embodiments, R³ is

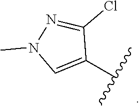 [SMILES: *c1cn(C)nc1Cl]

In some embodiments, R³ is

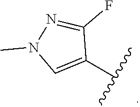 [SMILES: *c1cn(C)nc1F]

In some embodiments, R³ is

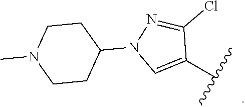 [SMILES: *c1cn(C2CCN(C)CC2)nc1Cl]

In some embodiments, R³ is

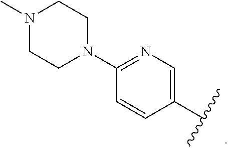 [SMILES: *c1ccc(N2CCN(C)CC2)nc1]

In some embodiments, R³ is

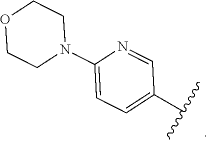 [SMILES: *c1ccc(N2CCOCC2)nc1]

In some embodiments, R³ is selected from:

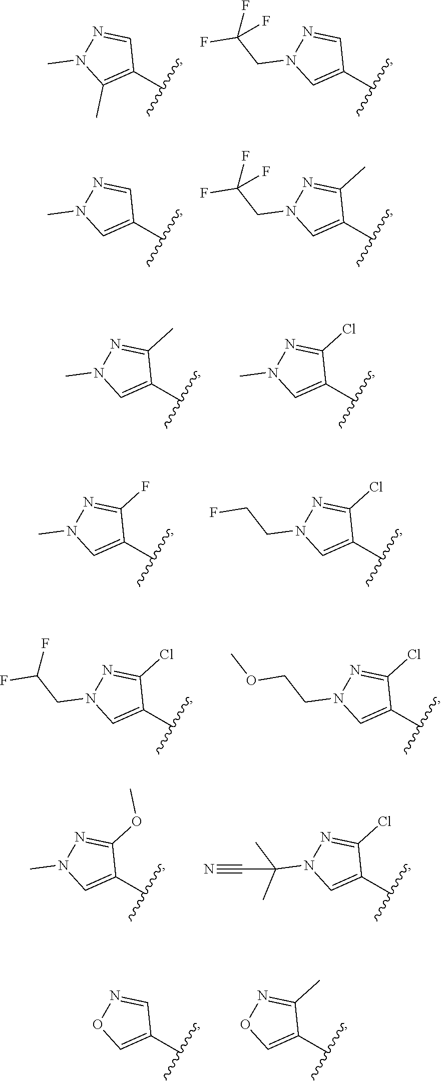

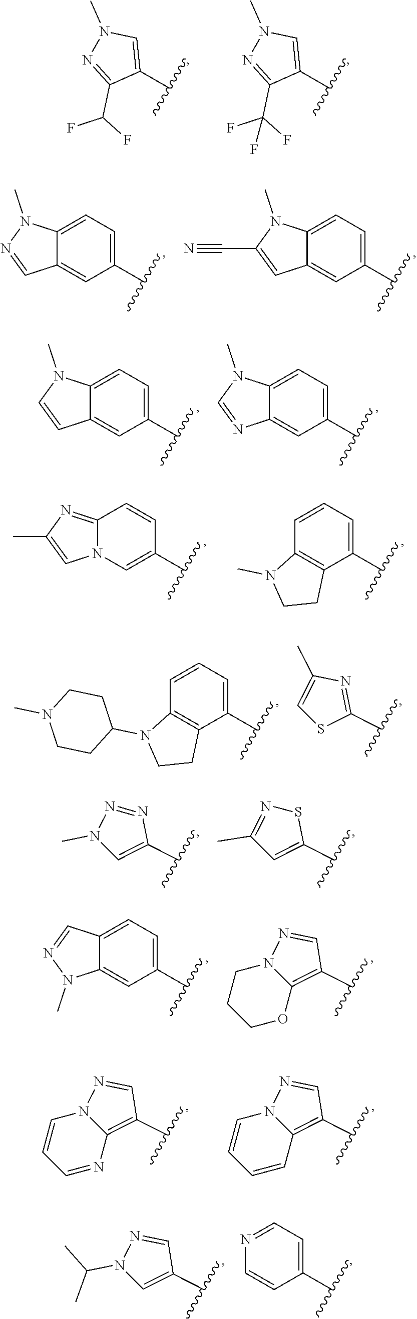

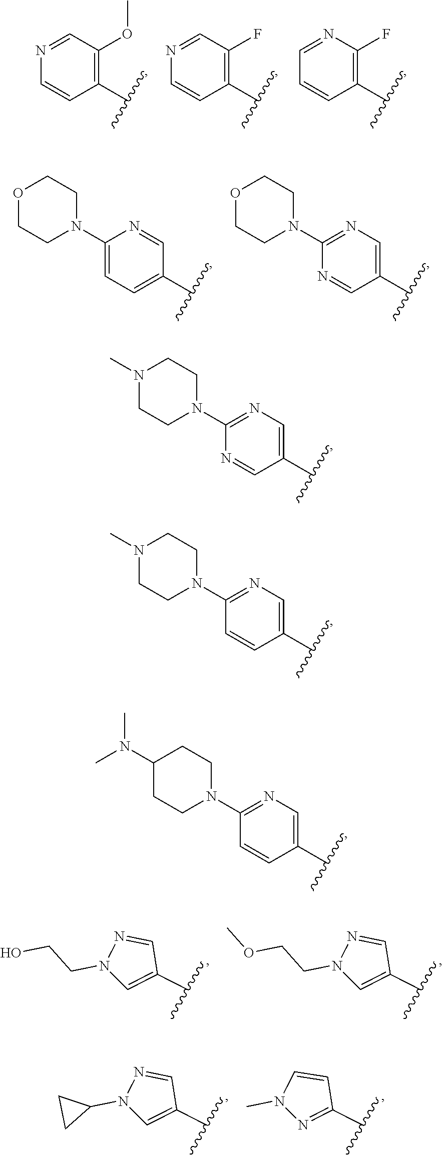

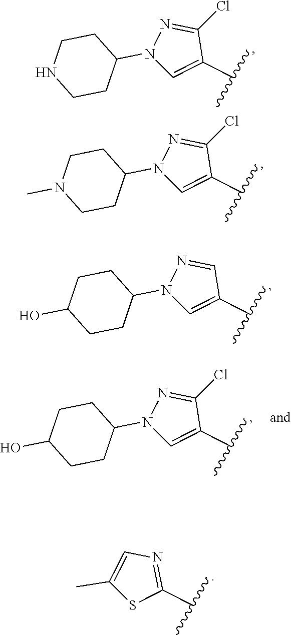

In some embodiments, R³ is selected from:

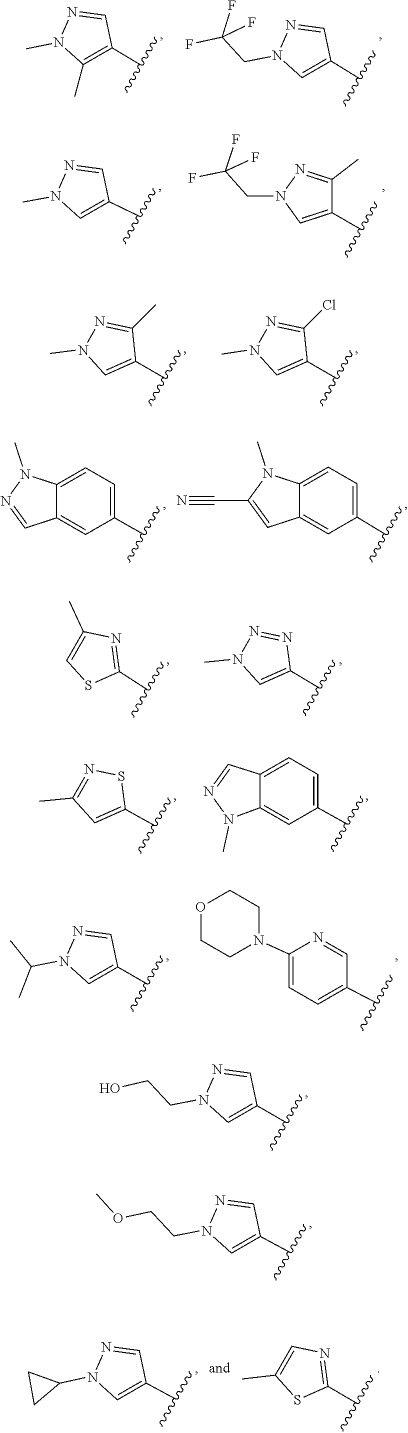

In some embodiments, R³ is selected from:

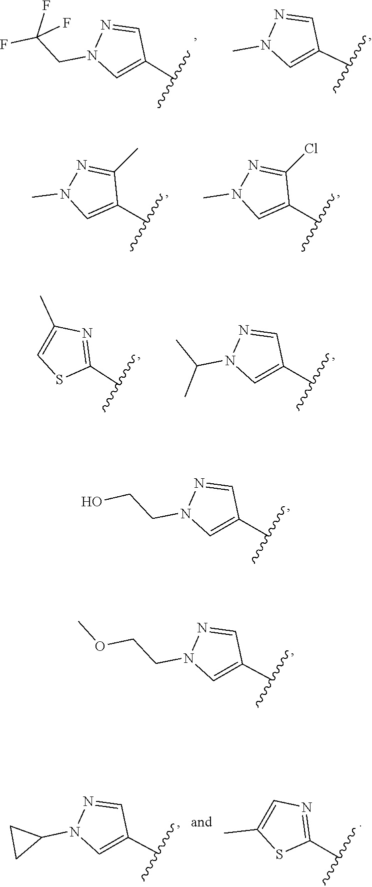

In some embodiments, R³ is

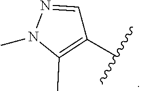 [SMILES: *c1cnn(C)c1C]

In some embodiments, R³ is

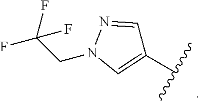 [SMILES: *c1cnn(CC(F)(F)F)c1]

In some embodiments, R³ is

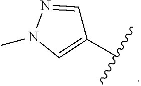 [SMILES: *c1cnn(C)c1]

In some embodiments, R³ is

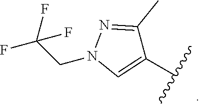 [SMILES: *c1cn(CC(F)(F)F)nc1C]

In some embodiments, R³ is

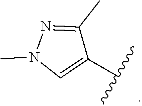 [SMILES: *c1cn(C)nc1C]

In some embodiments, R³ is

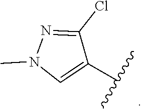 [SMILES: *c1cn(C)nc1Cl]

In some embodiments, R³ is

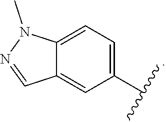 [SMILES: *c1ccc2c(cnn2C)c1]

In some embodiments, R³ is

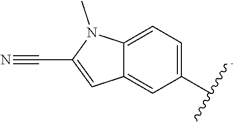 [SMILES: *c1ccc2c(c1)cc(C#N)n2C]

In some embodiments, R³ is

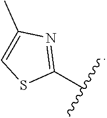 [SMILES: *c1nc(C)cs1]

In some embodiments, R³ is

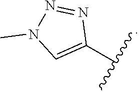 [SMILES: *c1cn(C)nn1]

In some embodiments, R³ is

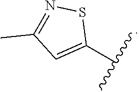 [SMILES: *c1cc(C)ns1]

In some embodiments, R³ is

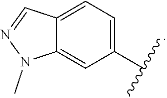 [SMILES: *c1ccc2cnn(C)c2c1]

In some embodiments, R³ is

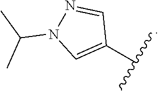 [SMILES: *c1cnn(C(C)C)c1]

In some embodiments, R³ is

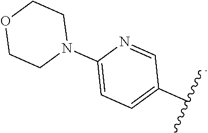 [SMILES: *c1ccc(N2CCOCC2)nc1]

In some embodiments, R³ is

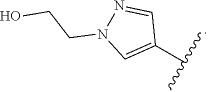 [SMILES: *c1cnn(CCO)c1]

In some embodiments, R³ is

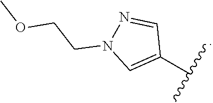 [SMILES: *c1cnn(CCOC)c1]

In some embodiments, R³ is

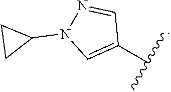 [SMILES: *c1cnn(C2CC2)c1]

In some embodiments, R³ is

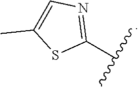 [SMILES: *c1ncc(C)s1]

In some embodiments, R³ is

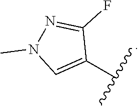 [SMILES: *c1cn(C)nc1F]

In some embodiments, R³ is

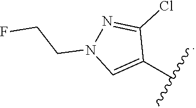 [SMILES: *c1cn(CCF)nc1Cl]

In some embodiments, R³ is

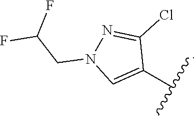 [SMILES: *c1cn(CC(F)F)nc1Cl]

In some embodiments, R³ is

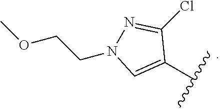 [SMILES: *c1cn(CCOC)nc1Cl]

In some embodiments, R³ is

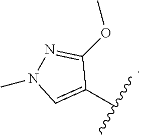 [SMILES: *c1cn(C)nc1OC]

In some embodiments, R³ is

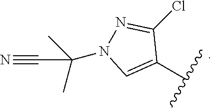 [SMILES: *c1cn(C(C)(C)C#N)nc1Cl]

In some embodiments, R³ is

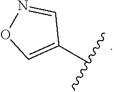 [SMILES: *c1cnoc1]

In some embodiments, R³ is

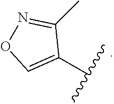 [SMILES: *c1conc1C]

In some embodiments, R³ is

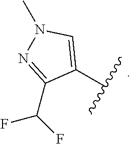 [SMILES: *c1cn(C)nc1C(F)F]

In some embodiments, R³ is

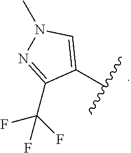 [SMILES: *c1cn(C)nc1C(F)(F)F]

In some embodiments, R³ is

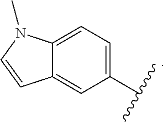 [SMILES: *c1ccc2c(ccn2C)c1]

In some embodiments, R³ is

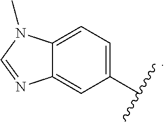 [SMILES: *c1ccc2c(c1)ncn2C]

In some embodiments, R³ is

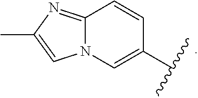 [SMILES: *c1ccc2nc(C)cn2c1]

In some embodiments, R³ is

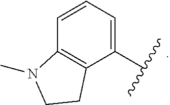 [SMILES: *c1cccc2c1CCN2C]

In some embodiments, R³ is

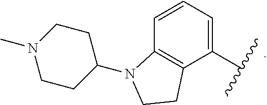 [SMILES: *c1cccc2c1CCN2C1CCN(C)CC1]

In some embodiments, R³ is

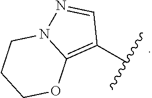 [SMILES: *c1cnn2c1OCCC2]

In some embodiments R³ is

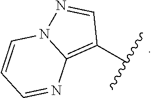 [SMILES: *c1cnn2cccnc12]

In some embodiments, R³ is

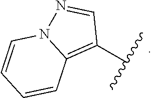 [SMILES: *c1cnn2ccccc12]

In some embodiments, R³ is

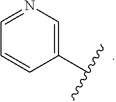 [SMILES: *c1cccnc1]

In some embodiments, R³ is

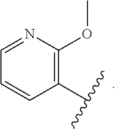 [SMILES: *c1cccnc1OC]

In some embodiments, R³ is

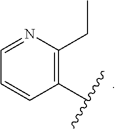 [SMILES: *c1cccnc1CC]

In some embodiments, R³ is

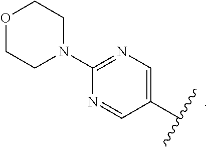 [SMILES: *c1cnc(N2CCOCC2)nc1]

In some embodiments, R³ is

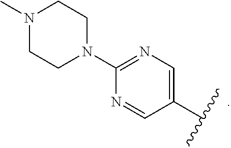 [SMILES: *c1cnc(N2CCN(C)CC2)nc1]

In some embodiments, R³ is

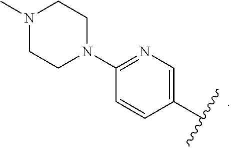 [SMILES: *c1ccc(N2CCN(C)CC2)nc1]

In some embodiments, R³ is

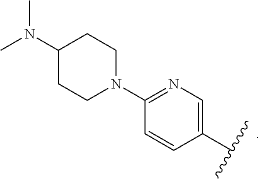 [SMILES: *c1ccc(N2CCC(N(C)C)CC2)nc1]

In some embodiments, R³ is

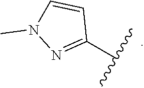 [SMILES: *c1ccn(C)n1]

In some embodiments, R³ is

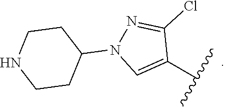 [SMILES: *c1cn(C2CCNCC2)nc1Cl]

In some embodiments, R³ is

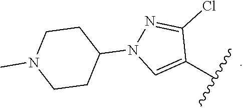 [SMILES: *c1cn(C2CCN(C)CC2)nc1Cl]

In some embodiments, R³ is

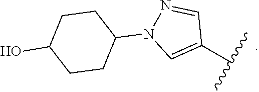 [SMILES: *c1cnn(C2CCC(O)CC2)c1]

In some embodiments, R³ is

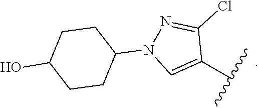 [SMILES: *c1cn(C2CCC(O)CC2)nc1Cl]

In some embodiments, each R¹² is independently aryl, heteroaryl, alkyl, heteroalkyl, haloalkyl, halo, cyano, alkoxy, heterocycloalkyl, —N(R¹³)₂, —S(═O)₂NH₂, —S(═O)₂alkyl, —S(═O)₂aryl, —S(═O)₂heteroaryl, or cycloalkyl. In some embodiments, each R¹² is independently aryl, heteroaryl, alkyl, heteroalkyl, haloalkyl, halo, cyano, alkoxy, heterocycloalkyl, —N(R¹³)₂, —S(═O)₂NH₂, or cycloalkyl. In some embodiments, each R¹² is independently alkyl, heteroalkyl, haloalkyl, halo, cyano, heterocycloalkyl, —N(R¹³)₂, or cycloalkyl. In some embodiments, each R¹² is independently aryl. In some embodiments, each R¹² is independently heteroaryl. In some embodiments, each R¹² is independently alkyl. In some embodiments, each R¹² is independently heteroalkyl. In some embodiments, each R¹² is independently haloalkyl. In some embodiments, each R¹² is independently halo. In some embodiments, each R¹² is cyano. In some embodiments, each R¹² is independently alkoxy. In some embodiments, each R¹² is independently heterocycloalkyl. In some embodiments, each R¹² is independently —N(R¹³)₂. In some embodiments, each R¹² is independently —S(═O)₂NH₂. In some embodiments, each R¹² is independently —S(═O)₂alkyl. In some embodiments, each R¹² is independently —S(═O)₂aryl. In some embodiments, each R¹² is independently —S(═O)₂heteroaryl. In some embodiments, each R¹² is independently cycloalkyl. In some embodiments, each R¹² is independently methyl, ethyl, n-propyl, iso-propyl, n-butyl, iso-butyl, sec-butyl, tert-butyl, hydroxyethyl, methoxyethyl, trifluoromethyl, trifluoroethyl, pentafluoroethyl, fluoro, chloro, cyano, methoxy, azetidinyl, oxetanyl, pyrrolidinyl, imidazolidinyl, tetrahydrofuranyl, piperidinyl, piperazinyl, tetrahydropyranyl, morpholinyl, —N(R¹³)₂, cyclopropyl, cyclobutyl, cyclopentyl, or cyclohexyl. In some embodiments, each R¹² is independently methyl, iso-propyl, tert-butyl, hydroxyethyl, methoxyethyl, trifluoromethyl, trifluoroethyl, fluoro, chloro, cyano, methoxy, oxetanyl, piperidinyl, piperazinyl, morpholinyl, or cyclopropyl. In some embodiments, each R¹² is independently methyl, fluoro, chloro, methoxy, oxetanyl, piperidinyl, piperazinyl, or morpholinyl. In some embodiments, each R¹² is independently methyl, hydroxyethyl, methoxyethyl, trifluoroethyl, or chloro. In some embodiments, each R¹² is independently methyl or chloro. In some embodiments, each R¹² is methyl. In some embodiments, each R¹² is ethyl. In some embodiments, each R¹² is n-propyl. In some embodiments, each R¹² is iso-propyl. In some embodiments, each R¹² is n-butyl. In some embodiments, each R¹² is iso-butyl. In some embodiments, each R¹² is sec-butyl. In some embodiments, each R¹² is tert-butyl. In some embodiments, each R¹² is hydroxyethyl. In some embodiments, each R¹² is methoxyethyl. In some embodiments, each R¹² is trifluoromethyl. In some embodiments, each R¹² is trifluoroethyl. In some embodiments, each R¹² is pentafluoroethyl. In some embodiments, each R¹² is fluoro. In some embodiments, each R¹² is chloro. In some embodiments, each R¹² is cyano. In some embodiments, each R¹² is methoxy. In some embodiments, each R¹² is azetidinyl. In some embodiments, each R¹² is oxetanyl. In some embodiments, each R¹² is pyrrolidinyl. In some embodiments, each R¹² is imidazolidinyl. In some embodiments, each R¹² is tetrahydrofuranyl. In some embodiments, each R¹² is piperidinyl. In some embodiments, each R¹² is piperazinyl. In some embodiments, each R¹² is tetrahydropyranyl. In some embodiments, each R¹² is morpholinyl. In some embodiments, each R¹² is cyclopropyl. In some embodiments, each R¹² is cyclobutyl. In some embodiments, each R¹² is cyclopentyl. In some embodiments, each R¹² is cyclohexyl.

In some embodiments, each R¹³ is independently alkyl, cycloalkyl, aryl, or heteroaryl. In some embodiments, each R¹³ is independently alkyl or cycloalkyl. In some embodiments, each R¹³ is independently alkyl. In some embodiments, each R¹³ is independently cycloalkyl. In some embodiments, each R¹³ is independently aryl. In some embodiments, each R¹³ is independently heteroaryl. In some embodiments, each R¹³ is independently methyl, ethyl, n-propyl, iso-propyl, n-butyl, iso-butyl, sec-butyl, tert-butyl, cyclopropyl, cyclobutyl, cyclopentyl, or cyclohexyl. In some embodiments, each R¹³ is independently methyl, ethyl, iso-propyl, tert-butyl, cyclopropyl, cyclopentyl, or cyclohexyl. In some embodiments, each R¹³ is independently methyl, cyclopropyl, or cyclohexyl. In some embodiments, each R¹³ is methyl. In some embodiments, each R¹³ is ethyl. In some embodiments, each R¹³ is n-propyl. In some embodiments, each R¹³ is iso-propyl. In some embodiments, each R¹³ is n-butyl. In some embodiments, each R¹³ is iso-butyl. In some embodiments, each R¹³ is sec-butyl. In some embodiments, each R¹³ is tert-butyl. In some embodiments, each R¹³ is cyclopropyl. In some embodiments, each R¹³ is cyclobutyl. In some embodiments, each R¹³ is cyclopentyl. In some embodiments, each R¹³ is cyclohexyl.

In some embodiments, the aryl, heteroaryl, heterocycloalkyl, or cycloalkyl of R¹² is unsubstituted. In some embodiments, the aryl, heteroaryl, heterocycloalkyl, or cycloalkyl of R¹² is substituted with 1 or 2 R¹⁴. In some embodiments, the aryl, heteroaryl, heterocycloalkyl, or cycloalkyl of R¹² is substituted with 1 R¹⁴. In some embodiments, the aryl, heteroaryl, heterocycloalkyl, or cycloalkyl of R¹² is substituted with 2 R¹⁴.

In some embodiments, each R¹⁴ is independently aryl, heteroaryl, alkyl, cycloalkyl, heterocycloalkyl, halo, heteroalkyl, haloalkyl, cyano, hydroxy, amino, —N(R¹⁵)₂, —S(═O)₂alkyl, —S(═O)₂aryl, —S(═O)₂heteroaryl, or alkoxy. In some embodiments, each R¹⁴ is independently alkyl, cycloalkyl, heterocycloalkyl, halo, cyano, —N(R¹⁵)₂, or alkoxy. In some embodiments, each R¹⁴ is independently aryl. In some embodiments, each R¹⁴ is independently heteroaryl. In some embodiments, each R¹⁴ is independently alkyl. In some embodiments, each R¹⁴ is independently cycloalkyl. In some embodiments, each R¹⁴ is independently heterocycloalkyl. In some embodiments, each R¹⁴ is independently halo. In some embodiments, each R¹⁴ is independently heteroalkyl. In some embodiments, each R¹⁴ is independently haloalkyl. In some embodiments, each R¹⁴ is cyano. In some embodiments, each R¹⁴ is hydroxy. In some embodiments, each R¹⁴ is amino. In some embodiments, each R¹⁴ is independently —N(R¹⁵)₂. In some embodiments, each R¹⁴ is independently —S(═O)₂alkyl. In some embodiments, each R¹⁴ is independently —S(═O)₂aryl. In some embodiments, each R¹⁴ is independently —S(═O)₂heteroaryl. In some embodiments, each R¹⁴ is independently alkoxy. In some embodiments, each R¹⁴ is independently methyl, ethyl, n-propyl, iso-propyl, n-butyl, iso-butyl, sec-butyl, tert-butyl, cyclopropyl, cyclobutyl, cyclopentyl, cyclohexyl, azetidinyl, oxetanyl, pyrrolidinyl, imidazolidinyl, tetrahydrofuranyl, piperidinyl, piperazinyl, tetrahydropyranyl, morpholinyl, fluoro, chloro, cyano, —N(R¹⁵)₂ methoxy, ethoxy, or trifluoromethoxy. In some embodiments, each R¹⁴ is independently methyl, ethyl, iso-propyl, tert-butyl, pyrrolidinyl, piperidinyl, morpholinyl, fluoro, chloro, —N(R¹⁵)₂, or methoxy. In some embodiments, each R¹⁴ is methyl. In some embodiments, each R¹⁴ is ethyl. In some embodiments, each R¹⁴ is n-propyl. In some embodiments, each R¹⁴ is iso-propyl. In some embodiments, each R¹⁴ is n-butyl. In some embodiments, each R¹⁴ is iso-butyl. In some embodiments, each R¹⁴ is sec-butyl. In some embodiments, each R¹⁴ is tert-butyl. In some embodiments, each R¹⁴ is cyclopropyl. In some embodiments, each R¹⁴ is cyclobutyl. In some embodiments, each R¹⁴ is cyclopentyl. In some embodiments, each R¹⁴ is cyclohexyl. In some embodiments, each R¹⁴ is azetidinyl. In some embodiments, each R¹⁴ is oxetanyl. In some embodiments, each R¹⁴ is pyrrolidinyl. In some embodiments, each R¹⁴ is imidazolidinyl. In some embodiments, each R¹⁴ is tetrahydrofuranyl. In some embodiments, each R¹⁴ is piperidinyl. In some embodiments, each R¹⁴ is piperazinyl. In some embodiments, each R¹⁴ is tetrahydropyranyl. In some embodiments, each R¹⁴ is morpholinyl. In some embodiments, each R¹⁴ is fluoro. In some embodiments, each R¹⁴ is chloro. In some embodiments, each R¹⁴ is methoxy. In some embodiments, each R¹⁴ is ethoxy. In some embodiments, each R¹⁴ is trifluoromethoxy.

In some embodiments, each R¹⁵ is independently alkyl, cycloalkyl, aryl, or heteroaryl. In some embodiments, each R¹⁵ is independently alkyl or cycloalkyl. In some embodiments, each R¹⁵ is methyl, ethyl, n-propyl, iso-propyl, n-butyl, iso-butyl, sec-butyl, tert-butyl, cyclopropyl, cyclobutyl, cyclopentyl, or cyclohexyl. In some embodiments, each R¹⁵ is independently methyl, ethyl, iso-propyl, tert-butyl, cyclopropyl, cyclopentyl, or cyclohexyl. In some embodiments, each R¹⁵ is independently methyl, cyclopropyl, or cyclohexyl. In some embodiments, each R¹⁵ is methyl. In some embodiments, each R¹⁵ is ethyl. In some embodiments, each R¹⁵ is n-propyl. In some embodiments, each R¹⁵ is iso-propyl. In some embodiments, each R¹⁵ is n-butyl. In some embodiments, each R¹⁵ is iso-butyl. In some embodiments, each R¹⁵ is sec-butyl. In some embodiments, each R¹⁵ is tert-butyl. In some embodiments, each R¹⁵ is cyclopropyl. In some embodiments, each R¹⁵ is cyclobutyl. In some embodiments, each R¹⁵ is cyclopentyl. In some embodiments, each R¹⁵ is cyclohexyl.

In some embodiments, each R¹⁶ is independently aryl, heteroaryl, alkyl, heteroalkyl, haloalkyl, halo, cyano, hydroxy, amino, alkoxy, heterocycloalkyl, —N(R¹⁷)₂, —S(═O)₂NH₂, —S(═O)₂NMe₂, —S(═O)₂alkyl, —S(═O)₂aryl, —S(═O)₂heteroaryl, or cycloalkyl. In some embodiments, each R¹⁶ is independently aryl, heteroaryl, alkyl, heteroalkyl, haloalkyl, halo, cyano, alkoxy, heterocycloalkyl, —N(R¹⁷)₂, —S(═O)₂NH₂, or cycloalkyl. In some embodiments, each R¹⁶ is independently alkyl, heteroalkyl, haloalkyl, halo, cyano, heterocycloalkyl, —N(R¹⁷)₂, or cycloalkyl. In some embodiments, each R¹⁶ is independently aryl. In some embodiments, each R¹⁶ is independently heteroaryl. In some embodiments, each R¹⁶ is independently alkyl. In some embodiments, each R¹⁶ is independently heteroalkyl. In some embodiments, each R¹⁶ is independently haloalkyl. In some embodiments, each R¹⁶ is independently halo. In some embodiments, each R¹⁶ is cyano. In some embodiments, each R¹⁶ is independently hydroxy. In some embodiments, each R¹⁶ is independently amino. In some embodiments, each R¹⁶ is independently alkoxy. In some embodiments, each R¹⁶ is independently heterocycloalkyl. In some embodiments, each R¹⁶ is independently —N(R¹⁷)₂. In some embodiments, each R¹⁶ is independently —S(═O)₂NH₂. In some embodiments, each R¹⁶ is independently —S(═O)₂NMe₂. In some embodiments, each R¹⁶ is independently —S(═O)₂alkyl. In some embodiments, each R¹⁶ is independently —S(═O)₂aryl. In some embodiments, each R¹⁶ is independently —S(═O)₂heteroaryl. In some embodiments, each R¹⁶ is independently cycloalkyl. In some embodiments, each R¹⁶ is independently methyl, ethyl, n-propyl, iso-propyl, n-butyl, iso-butyl, sec-butyl, tert-butyl, hydroxyethyl, methoxyethyl, trifluoromethyl, trifluoroethyl, pentafluoroethyl, fluoro, chloro, cyano, methoxy, azetidinyl, oxetanyl, pyrrolidinyl, imidazolidinyl, tetrahydrofuranyl, piperidinyl, piperazinyl, tetrahydropyranyl, morpholinyl, —N(R¹⁷)₂, cyclopropyl, cyclobutyl, cyclopentyl, or cyclohexyl. In some embodiments, each R¹⁶ is independently methyl, iso-propyl, tert-butyl, hydroxyethyl, methoxyethyl, trifluoromethyl, trifluoroethyl, fluoro, chloro, cyano, methoxy, oxetanyl, piperidinyl, piperazinyl, morpholinyl, or cyclopropyl. In some embodiments, each R¹⁶ is independently methyl, fluoro, chloro, methoxy, oxetanyl, piperidinyl, piperazinyl, or morpholinyl. In some embodiments, each R¹⁶ is independently methyl, hydroxyethyl, methoxyethyl, trifluoroethyl, or chloro. In some embodiments, each R¹⁶ is independently methyl or chloro. In some embodiments, each R¹⁶ is methyl. In some embodiments, each R¹⁶ is ethyl. In some embodiments, each R¹⁶ is n-propyl. In some embodiments, each R¹⁶ is iso-propyl. In some embodiments, each R¹⁶ is n-butyl. In some embodiments, each R¹⁶ is iso-butyl. In some embodiments, each R¹⁶ is sec-butyl. In some embodiments, each R¹⁶ is tert-butyl. In some embodiments, each R¹⁶ is hydroxyethyl. In some embodiments, each R¹⁶ is methoxyethyl. In some embodiments, each R¹⁶ is trifluoromethyl. In some embodiments, each R¹⁶ is trifluoroethyl. In some embodiments, each R¹⁶ is pentafluoroethyl. In some embodiments, each R¹⁶ is fluoro. In some embodiments, each R¹⁶ is chloro. In some embodiments, each R¹⁶ is cyano. In some embodiments, each R¹⁶ is methoxy. In some embodiments, each R¹⁶ is azetidinyl. In some embodiments, each R¹⁶ is oxetanyl. In some embodiments, each R¹⁶ is pyrrolidinyl. In some embodiments, each R¹⁶ is imidazolidinyl. In some embodiments, each R¹⁶ is tetrahydrofuranyl. In some embodiments, each R¹⁶ is piperidinyl. In some embodiments, each R¹⁶ is piperazinyl. In some embodiments, each R¹⁶ is tetrahydropyranyl. In some embodiments, each R¹⁶ is morpholinyl. In some embodiments, each R¹⁶ is cyclopropyl. In some embodiments, each R¹⁶ is cyclobutyl. In some embodiments, each R¹⁶ is cyclopentyl. In some embodiments, each R¹⁶ is cyclohexyl.

In some embodiments, each R¹⁷ is independently alkyl, cycloalkyl, aryl, or heteroaryl. In some embodiments, each R¹⁷ is independently alkyl or cycloalkyl. In some embodiments, each R¹⁷ is independently alkyl. In some embodiments, each R¹⁷ is independently cycloalkyl. In some embodiments, each R¹⁷ is independently aryl. In some embodiments, each R¹⁷ is independently heteroaryl. In some embodiments, each R¹⁷ is independently methyl, ethyl, n-propyl, iso-propyl, n-butyl, iso-butyl, sec-butyl, tert-butyl, cyclopropyl, cyclobutyl, cyclopentyl, or cyclohexyl. In some embodiments, each R¹⁷ is independently methyl, ethyl, iso-propyl, tert-butyl, cyclopropyl, cyclopentyl, or cyclohexyl. In some embodiments, each R¹⁷ is independently methyl, cyclopropyl, or cyclohexyl. In some embodiments, each R¹⁷ is methyl. In some embodiments, each R¹⁷ is ethyl. In some embodiments, each R¹⁷ is n-propyl. In some embodiments, each R¹⁷ is iso-propyl. In some embodiments, each R¹⁷ is n-butyl. In some embodiments, each R¹⁷ is iso-butyl. In some embodiments, each R¹⁷ is sec-butyl. In some embodiments, each R¹⁷ is tert-butyl. In some embodiments, each R¹⁷ is cyclopropyl. In some embodiments, each R¹⁷ is cyclobutyl. In some embodiments, each R¹⁷ is cyclopentyl. In some embodiments, each R¹⁷ is cyclohexyl.

In some embodiments, the aryl, heteroaryl, heterocycloalkyl, or cycloalkyl of R¹⁶ is unsubstituted. In some embodiments, the aryl, heteroaryl, heterocycloalkyl, or cycloalkyl of R¹⁶ is substituted with 1 or 2 R¹⁸. In some embodiments, the aryl, heteroaryl, heterocycloalkyl, or cycloalkyl of R¹⁶ is substituted with 1 R¹⁸. In some embodiments, the aryl, heteroaryl, heterocycloalkyl, or cycloalkyl of R¹⁶ is substituted with 2 R¹⁸.

In some embodiments, each R¹⁸ is independently aryl, heteroaryl, alkyl, cycloalkyl, heterocycloalkyl, halo, heteroalkyl, haloalkyl, cyano, hydroxy, amino, —N(R¹⁹)₂, —S(═O)₂alkyl, —S(═O)₂aryl, —S(═O)₂heteroaryl, or alkoxy. In some embodiments, each R¹⁸ is independently alkyl, cycloalkyl, heterocycloalkyl, halo, cyano, —N(R¹⁹)₂, or alkoxy. In some embodiments, each R¹⁸ is independently aryl. In some embodiments, each R¹⁸ is independently heteroaryl. In some embodiments, each R¹⁸ is independently alkyl. In some embodiments, each R¹⁸ is independently cycloalkyl. In some embodiments, each R¹⁸ is independently heterocycloalkyl. In some embodiments, each R¹⁸ is independently halo. In some embodiments, each R¹⁸ is independently heteroalkyl. In some embodiments, each R¹⁸ is independently haloalkyl. In some embodiments, each R¹⁸ is cyano. In some embodiments, each R¹⁸ is hydroxy. In some embodiments, each R¹⁸ is amino. In some embodiments, each R¹⁸ is independently —N(R¹⁹)₂. In some embodiments, each R¹⁸ is independently —S(═O)₂alkyl. In some embodiments, each R¹⁸ is independently —S(═O)₂aryl. In some embodiments, each R¹⁸ is independently —S(═O)₂heteroaryl. In some embodiments, each R¹⁸ is independently alkoxy. In some embodiments, each R¹⁸ is independently methyl, ethyl, n-propyl, iso-propyl, n-butyl, iso-butyl, sec-butyl, tert-butyl, cyclopropyl, cyclobutyl, cyclopentyl, cyclohexyl, azetidinyl, oxetanyl, pyrrolidinyl, imidazolidinyl, tetrahydrofuranyl, piperidinyl, piperazinyl, tetrahydropyranyl, morpholinyl, fluoro, chloro, cyano, —N(R¹⁹)₂ methoxy, ethoxy, or trifluoromethoxy. In some embodiments, each R¹⁸ is independently methyl, ethyl, iso-propyl, tert-butyl, pyrrolidinyl, piperidinyl, morpholinyl, fluoro, chloro, —N(R¹⁹)₂, or methoxy. In some embodiments, each R¹⁸ is methyl. In some embodiments, each R¹⁸ is ethyl. In some embodiments, each R¹⁸ is n-propyl. In some embodiments, each R¹⁸ is iso-propyl. In some embodiments, each R¹⁸ is n-butyl. In some embodiments, each R¹⁸ is iso-butyl. In some embodiments, each R¹⁸ is sec-butyl. In some embodiments, each R¹⁸ is tert-butyl. In some embodiments, each R¹⁸ is cyclopropyl. In some embodiments, each R¹⁸ is cyclobutyl. In some embodiments, each R¹⁸ is cyclopentyl. In some embodiments, each R¹⁸ is cyclohexyl. In some embodiments, each R¹⁸ is azetidinyl. In some embodiments, each R¹⁸ is oxetanyl. In some embodiments, each R¹⁸ is pyrrolidinyl. In some embodiments, each R¹⁸ is imidazolidinyl. In some embodiments, each R¹⁸ is tetrahydrofuranyl. In some embodiments, each R¹⁸ is piperidinyl. In some embodiments, each R¹⁸ is piperazinyl. In some embodiments, each R¹⁸ is tetrahydropyranyl. In some embodiments, each R¹⁸ is morpholinyl. In some embodiments, each R¹⁸ is fluoro. In some embodiments, each R¹⁸ is chloro. In some embodiments, each R¹⁸ is methoxy. In some embodiments, each R¹⁸ is ethoxy. In some embodiments, each R¹⁸ is trifluoromethoxy.

In some embodiments, each R¹⁹ is independently alkyl, cycloalkyl, aryl, or heteroaryl. In some embodiments, each R¹⁹ is independently alkyl or cycloalkyl. In some embodiments, each R¹⁹ is methyl, ethyl, n-propyl, iso-propyl, n-butyl, iso-butyl, sec-butyl, tert-butyl, cyclopropyl, cyclobutyl, cyclopentyl, or cyclohexyl. In some embodiments, each R¹⁹ is independently methyl, ethyl, iso-propyl, tert-butyl, cyclopropyl, cyclopentyl, or cyclohexyl. In some embodiments, each R¹⁹ is independently methyl, cyclopropyl, or cyclohexyl. In some embodiments, each R¹⁹ is methyl. In some embodiments, each R¹⁹ is ethyl. In some embodiments, each R¹⁹ is n-propyl. In some embodiments, each R¹⁹ is iso-propyl. In some embodiments, each R¹⁹ is n-butyl. In some embodiments, each R¹⁹ is iso-butyl. In some embodiments, each R¹⁹ is sec-butyl. In some embodiments, each R¹⁹ is tert-butyl. In some embodiments, each R¹⁹ is cyclopropyl. In some embodiments, each R¹⁹ is cyclobutyl. In some embodiments, each R¹⁹ is cyclopentyl. In some embodiments, each R¹⁹ is cyclohexyl.

In some embodiments, m is 0, 1, 2, 3, or 4. In some embodiments, m is 0, 1, 2, or 3. In some embodiments, m is 0, 1, 2, or 4. In some embodiments, m is 0, 2, 3, or 4. In some embodiments, m is 1, 2, 3, or 4. In some embodiments, m is 0, 1, or 2. In some embodiments, m is 0, 1, or 3. In some embodiments, m is 0, 1, or 4. In some embodiments, m is 0, 2, or 3. In some embodiments, m is 0, 2, or 4. In some embodiments, m is 0, 3, or 4. In some embodiments, m is 1, 2, or 3. In some embodiments, m is 1, 2, or 4. In some embodiments, m is 1, 3, or 4. In some embodiments, m is 2, 3, or 4. In some embodiments, m is 0 or 1. In some embodiments, m is 0 or 2. In some embodiments, m is 0 or 3. In some embodiments, m is 0 or 4. In some embodiments, m is 1 or 2. In some embodiments, m is 1 or 3. In some embodiments, m is 1 or 4. In some embodiments, m is 2 or 3. In some embodiments, m is 2 or 4. In some embodiments, m is 3 or 4. In some embodiments, m is 0. In some embodiments, m is 1. In some embodiments, m is 2. In some embodiments, m is 3. In some embodiments, m is 4.

In some embodiments, the compound of Formula I is selected from:

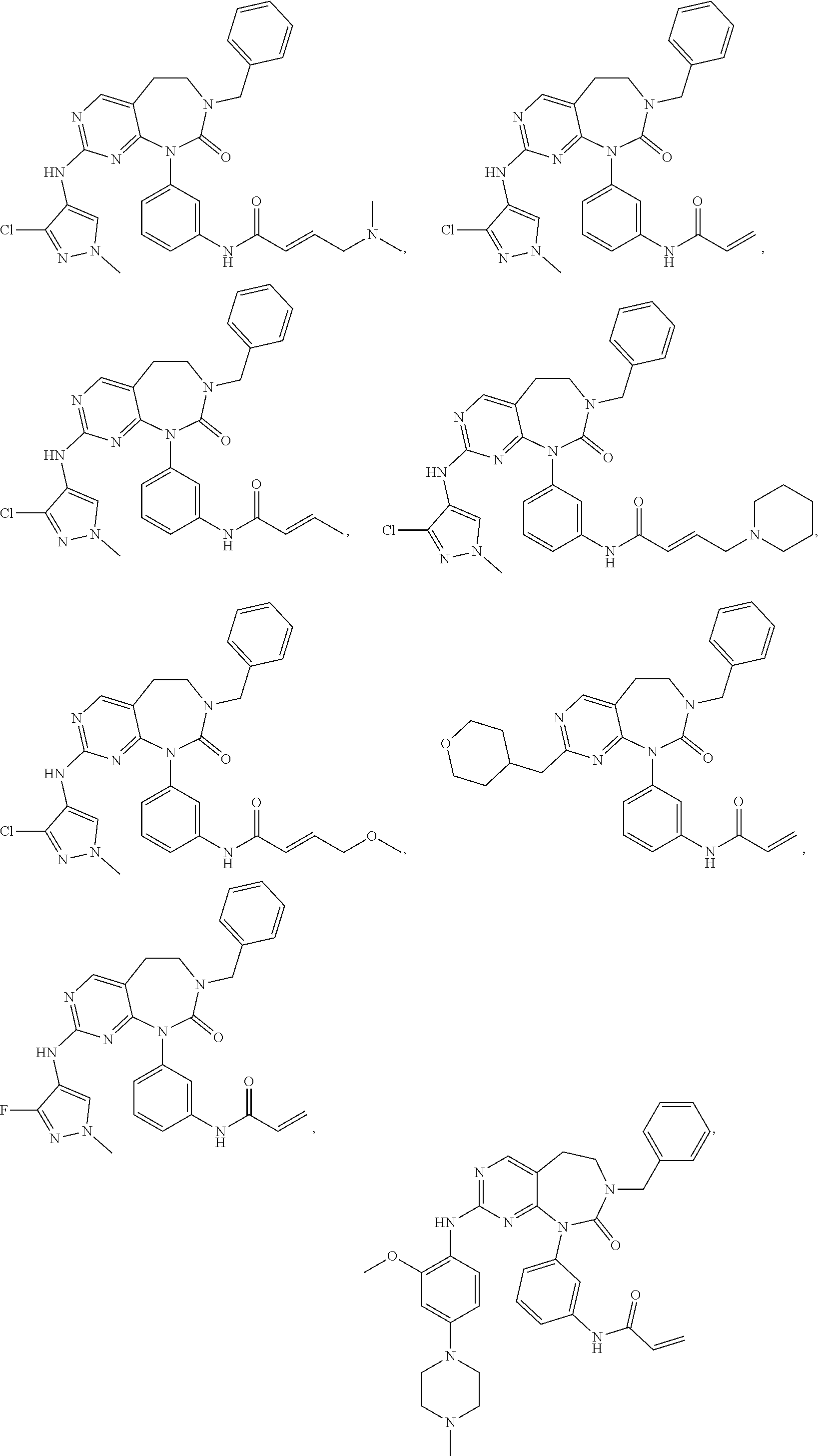

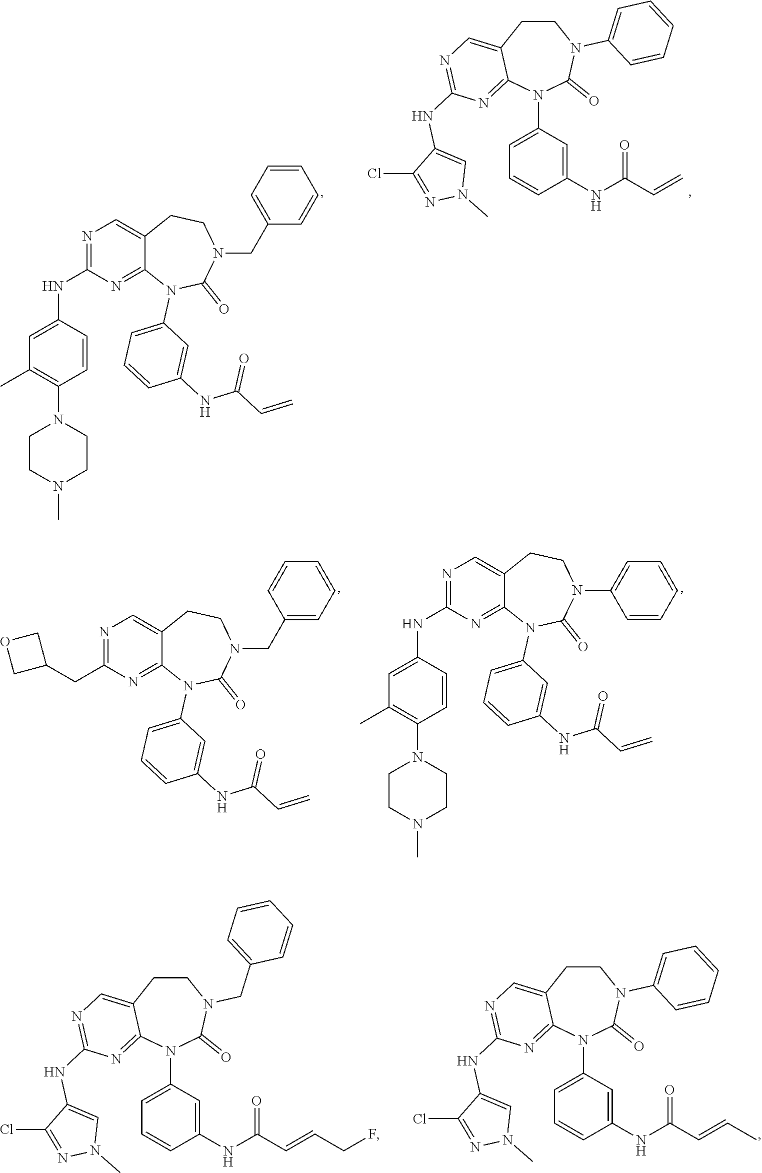 [SMILES: C=CC(=O)Nc1cccc(N2C(=O)N(Cc3ccccc3)CCc3cnc(Nc4ccc(N5CCN(C)CC5)c(C)c4)nc32)c1 ; C=CC(=O)Nc1cccc(N2C(=O)N(c3ccccc3)CCc3cnc(Nc4cn(C)nc4Cl)nc32)c1 ; C=CC(=O)Nc1cccc(N2C(=O)N(Cc3ccccc3)CCc3cnc(CC4COC4)nc32)c1 ; C=CC(=O)Nc1cccc(N2C(=O)N(c3ccccc3)CCc3cnc(Nc4ccc(N5CCN(C)CC5)c(C)c4)nc32)c1 ; Cn1cc(Nc2ncc3c(n2)N(c2cccc(NC(=O)/C=C/CF)c2)C(=O)N(Cc2ccccc2)CC3)c(Cl)n1 ; C/C=C/C(=O)Nc1cccc(N2C(=O)N(c3ccccc3)CCc3cnc(Nc4cn(C)nc4Cl)nc32)c1]

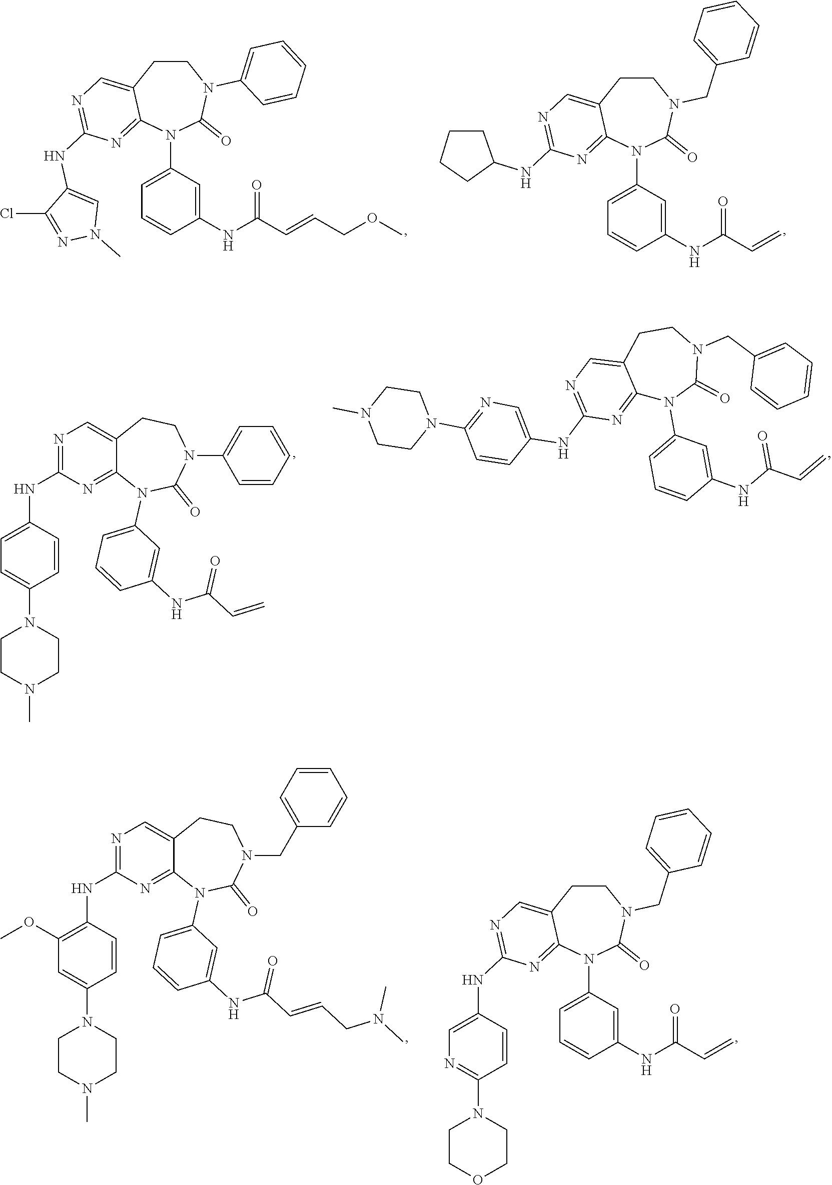

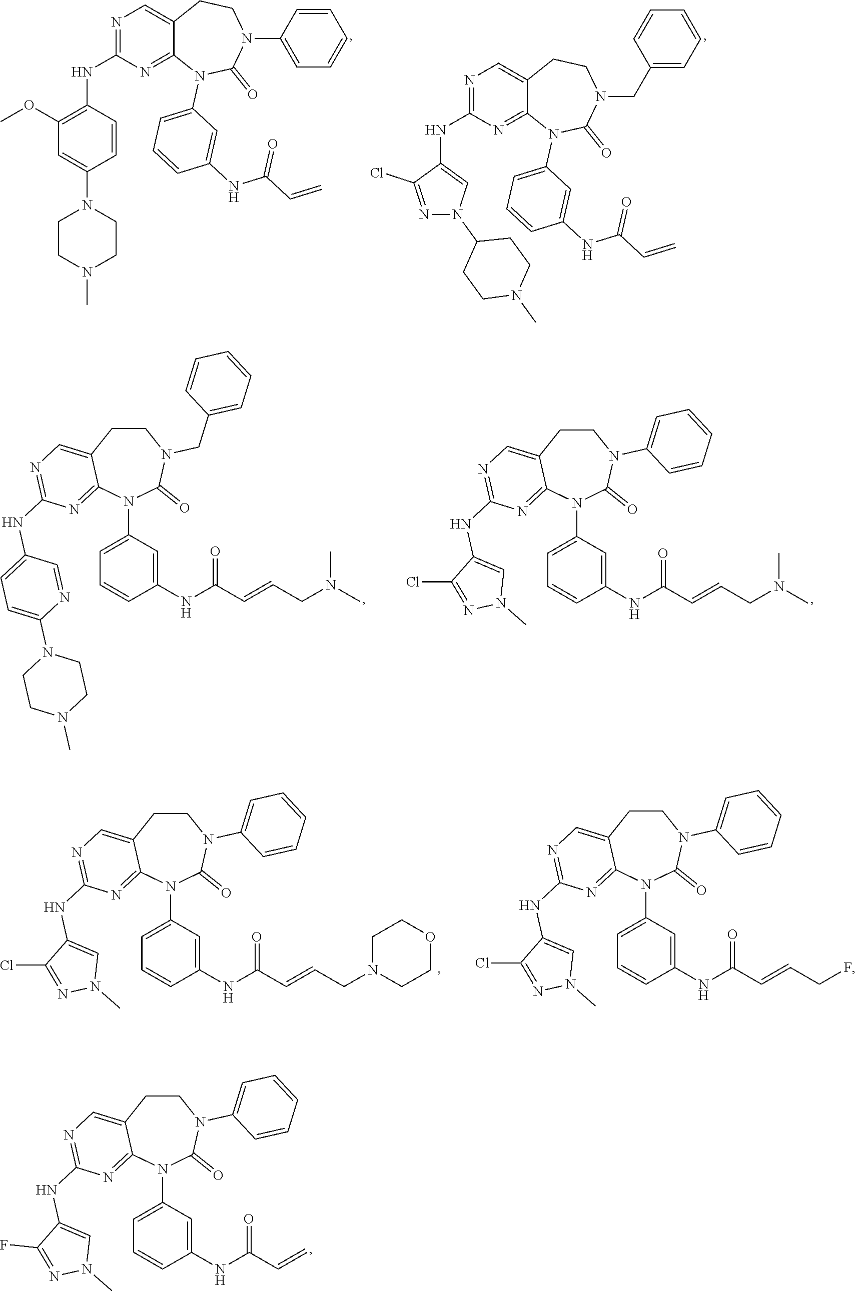

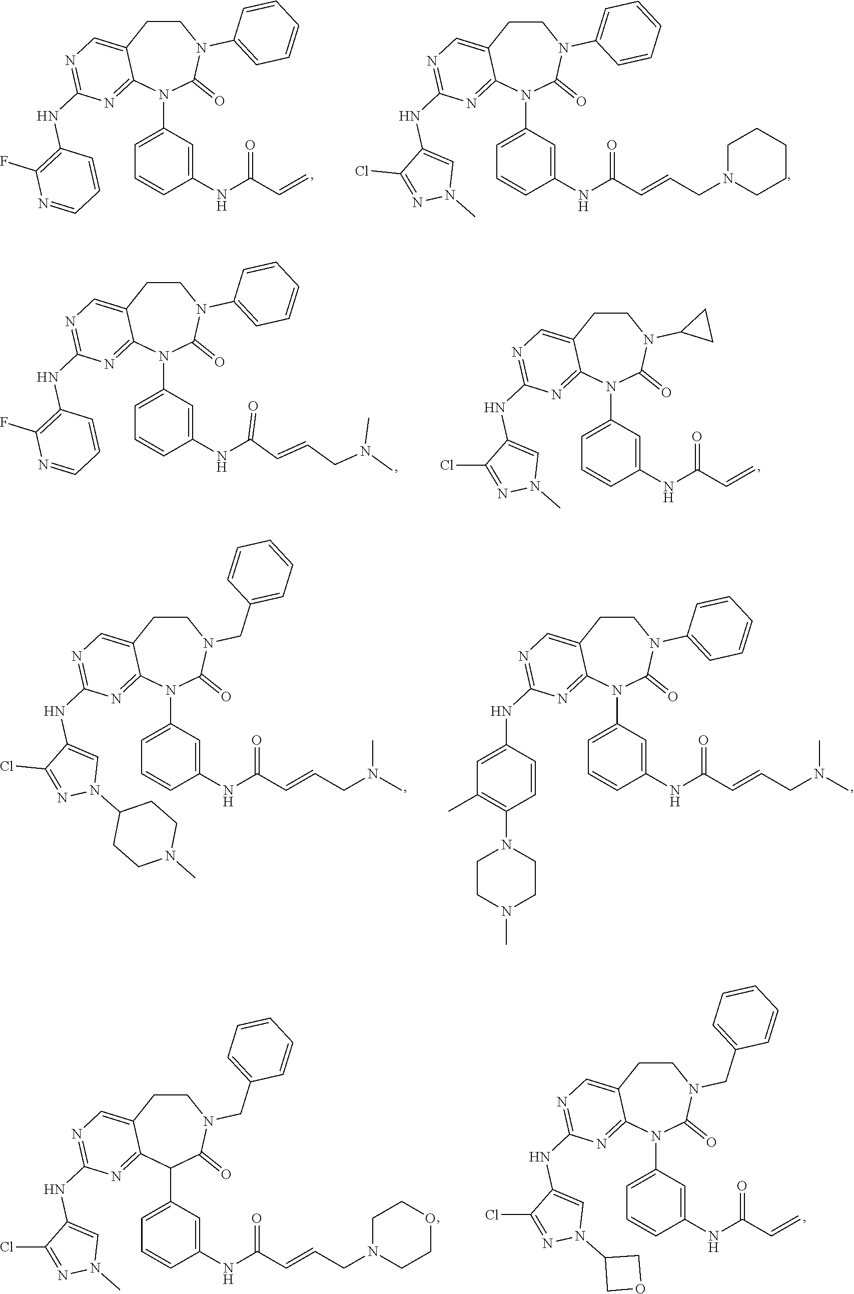

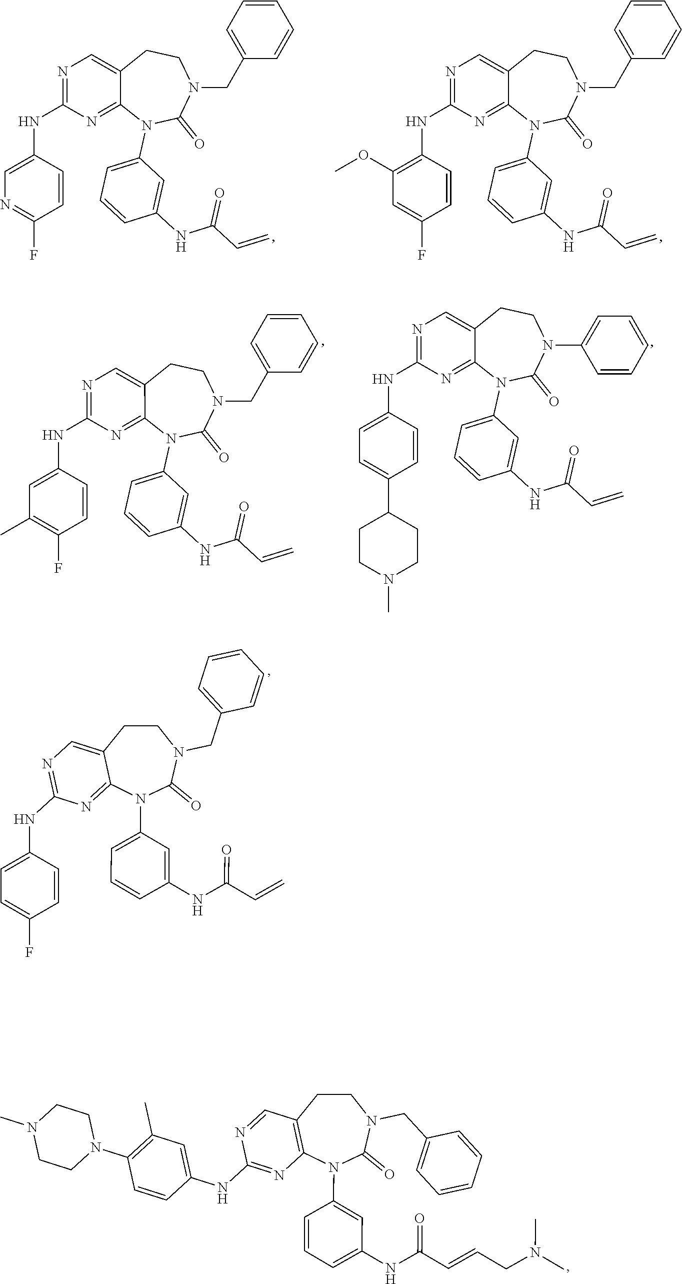

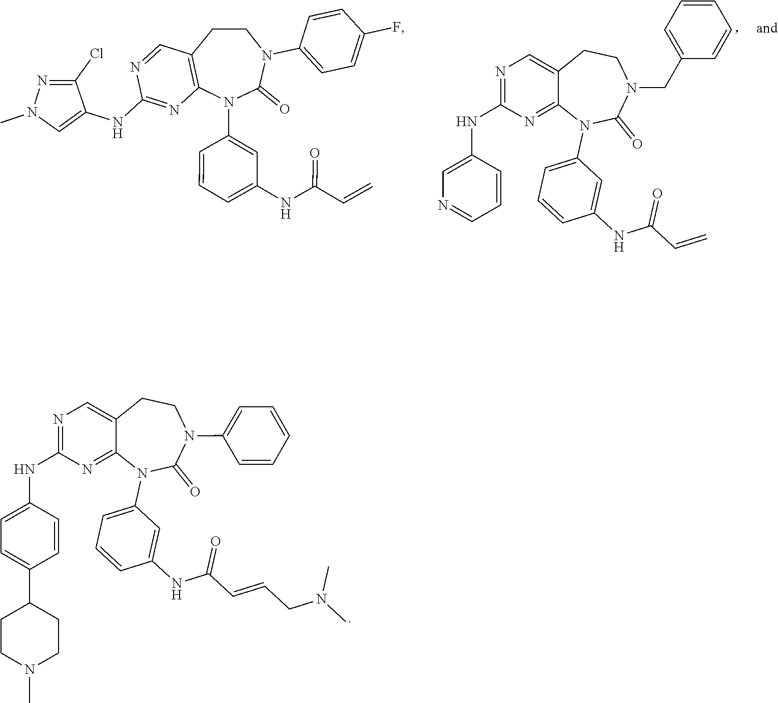 [SMILES: C=CC(=O)Nc1cccc(N2C(=O)N(c3ccc(F)cc3)CCc3cnc(Nc4cn(C)nc4Cl)nc32)c1 ; C=CC(=O)Nc1cccc(N2C(=O)N(Cc3ccccc3)CCc3cnc(Nc4cccnc4)nc32)c1 ; CN(C)C/C=C/C(=O)Nc1cccc(N2C(=O)N(c3ccccc3)CCc3cnc(Nc4ccc(C5CCN(C)CC5)cc4)nc32)c1]

In another aspect, the present disclosure provides a pharmaceutical composition comprising a compound of Formula I, or a pharmaceutically acceptable salt thereof, and a pharmaceutically acceptable carrier.

Particular embodiments of the present disclosure are compounds of Formula I or its stereoisomers, tautomers, pharmaceutically acceptable salts, polymorphs, solvates and hydrates thereof, selected from the group consisting of,
(E)-N-(3-(7-benzyl-2-((3-chloro-1-methyl-1H-pyrazol-4-yl)amino)-8-oxo-5,6,7,8-tetrahydro-9H-pyrimido[4,5-d][1,3]diazepin-9-yl)phenyl)-4-(dimethylamino)but-2-enamide (Compound 1),
(E)-N-(3-(7-benzyl-2-((3-chloro-1-methyl-1H-pyrazol-4-yl)amino)-8-oxo-5,6,7,8-tetrahydro-9H-pyrimido[4,5-d][1,3]diazepin-9-yl)phenyl)but-2-enamide (Compound 2),
(E)-N-(3-(7-benzyl-2-((3-chloro-1-methyl-1H-pyrazol-4-yl)amino)-8-oxo-5,6,7,8-tetrahydro-9H-pyrimido[4,5-d][1,3]diazepin-9-yl)phenyl)-4-(piperidin-1-yl)but-2-enamide (Compound 3),
(E)-N-(3-(7-benzyl-2-((3-chloro-1-methyl-1H-pyrazol-4-yl)amino)-8-oxo-5,6,7,8-tetrahydro-9H-pyrimido[4,5-d][1,3]diazepin-9-yl)phenyl)-4-methoxybut-2-enamide (Compound 4),
N-(3-(7-benzyl-2-((3-chloro-1-methyl-1H-pyrazol-4-yl)amino)-8-oxo-5,6,7,8-tetrahydro-9H-pyrimido[4,5-d][1,3]diazepin-9-yl)phenyl)acrylamide (Compound 5),
(E)-N-(3-(7-benzyl-2-((3-chloro-1-methyl-1H-pyrazol-4-yl)amino)-8-oxo-5,6,7,8-tetrahydro-9H-pyrimido[4,5-d][1,3]diazepin-9-yl)phenyl)-4-fluorobut-2-enamide (Compound 6),
N-(3-(7-benzyl-2-((3-chloro-1-(1-methylpiperidin-4-yl)-1H-pyrazol-4-yl)amino)-8-oxo-5,6,7,8-tetrahydro-9H-pyrimido[4,5-d][1,3]diazepin-9-yl)phenyl)acrylamide (Compound 7),
(E)-N-(3-(7-benzyl-2-((3-chloro-1-(1-methylpiperidin-4-yl)-1H-pyrazol-4-yl)amino)-8-oxo-5,6,7,8-tetrahydro-9H-pyrimido[4,5-d][1,3]diazepin-9-yl)phenyl)-4-(dimethylamino)but-2-enamide (Compound 8),
N-(3-(7-benzyl-2-((4-fluoro-3-methylphenyl)amino)-8-oxo-5,6,7,8-tetrahydro-9H-pyrimido[4,5-d][1,3]diazepin-9-yl)phenyl)acrylamide (Compound 9),
(E)-N-(3-(7-benzyl-2-((3-chloro-1-methyl-1H-pyrazol-4-yl)amino)-8-oxo-5,6,7,8-tetrahydro-9H-pyrimido[4,5-d][1,3]diazepin-9-yl)phenyl)-4-morpholinobut-2-enamide (Compound 10),
N-(3-(7-benzyl-2-((3-fluoro-1-methyl-1H-pyrazol-4-yl)amino)-8-oxo-5,6,7,8-tetrahydro-9H-pyrimido[4,5-d][1,3]diazepin-9-yl)phenyl)acrylamide (Compound 11),
N-(3-(7-benzyl-8-oxo-2-(pyridin-3-ylamino)-5,6,7,8-tetrahydro-9H-pyrimido[4,5-d][1,3]diazepin-9-yl)phenyl)acrylamide (Compound 12),
N-(3-(7-benzyl-2-(cyclopentylamino)-8-oxo-5,6,7,8-tetrahydro-9H-pyrimido[4,5-d][1,3]diazepin-9-yl)phenyl)acrylamide (Compound 13),
N-(3-(7-benzyl-2-((2-methoxy-4-(4-methylpiperazin-1-yl)phenyl)amino)-8-oxo-5,6,7,8-tetrahydro-9H-pyrimido[4,5-d][1,3]diazepin-9-yl)phenyl)acrylamide (Compound 14),
(E)-N-(3-(7-benzyl-2-((2-methoxy-4-(4-methylpiperazin-1-yl)phenyl)amino)-8-oxo-5,6,7,8-tetrahydro-9H-pyrimido[4,5-d][1,3]diazepin-9-yl)phenyl)-4-(dimethylamino)but-2-enamide (Compound 15),
N-(3-(7-benzyl-2-((6-(4-methylpiperazin-1-yl)pyridin-3-yl)amino)-8-oxo-5,6,7,8-tetrahydro-9H-pyrimido[4,5-d][1,3]diazepin-9-yl)phenyl)acrylamide (Compound 16),
(E)-N-(3-(7-benzyl-2-((6-(4-methylpiperazin-1-yl)pyridin-3-yl)amino)-8-oxo-5,6,7,8-tetrahydro-9H-pyrimido[4,5-d][1,3]diazepin-9-yl)phenyl)-4-(dimethylamino)but-2-enamide (Compound 17),
N-(3-(7-benzyl-2-((4-fluorophenyl)amino)-8-oxo-5,6,7,8-tetrahydro-9H-pyrimido[4,5-d][1,3]diazepin-9-yl)phenyl)acrylamide (Compound 18),
N-(3-(7-benzyl-2-((6-morpholinopyridin-3-yl)amino)-8-oxo-5,6,7,8-tetrahydro-9H-pyrimido[4,5-d][1,3]diazepin-9-yl)phenyl)acrylamide (Compound 19),
N-(3-(2-((3-chloro-1-methyl-1H-pyrazol-4-yl)amino)-7-cyclopropyl-8-oxo-5,6,7,8-tetrahydro-9H-pyrimido[4,5-d][1,3]diazepin-9-yl)phenyl)acrylamide (Compound 20),
N-(3-(7-benzyl-2-((4-fluoro-2-methoxyphenyl)amino)-8-oxo-5,6,7,8-tetrahydro-9H-pyrimido[4,5-d][1,3]diazepin-9-yl)phenyl)acrylamide (Compound 21),
N-(3-(7-benzyl-2-((3-methyl-4-(4-methylpiperazin-1-yl)phenyl)amino)-8-oxo-5,6,7,8-tetrahydro-9H-pyrimido[4,5-d][1,3]diazepin-9-yl)phenyl)acrylamide (Compound 22),
(E)-N-(3-(7-benzyl-2-((3-methyl-4-(4-methylpiperazin-1-yl)phenyl)amino)-8-oxo-5,6,7,8-tetrahydro-9H-pyrimido[4,5-d][1,3]diazepin-9-yl)phenyl)-4-(dimethylamino)but-2-enamide (Compound 23),
N-(3-(7-benzyl-2-((3-chloro-1-methyl-1H-pyrazol-4-yl)amino)-8-oxo-5,6,7,8-tetrahydro-9H-pyrimido[4,5-d][1,3]diazepin-9-yl)phenyl)-2-chloro-2-fluoroacetamide (Compound 24),
N-(3-(7-benzyl-8-oxo-2-((tetrahydro-2H-pyran-4-yl)amino)-5,6,7,8-tetrahydro-9H-pyrimido[4,5-d][1,3]diazepin-9-yl)phenyl)acrylamide (Compound 25),
N-(3-(7-benzyl-2-((6-fluoropyridin-3-yl)amino)-8-oxo-5,6,7,8-tetrahydro-9H-pyrimido[4,5-d][1,3]diazepin-9-yl)phenyl)acrylamide (Compound 26),
N-(3-(7-benzyl-2-(oxetan-3-ylamino)-8-oxo-5,6,7,8-tetrahydro-9H-pyrimido[4,5-d][1,3]diazepin-9-yl)phenyl)acrylamide (Compound 27),
N-(3-(7-benzyl-2-((3-chloro-1-(oxetan-3-yl)-1H-pyrazol-4-yl)amino)-8-oxo-5,6,7,8-tetrahydro-9H-pyrimido[4,5-d][1,3]diazepin-9-yl)phenyl)acrylamide (Compound 28),
N-(3-(2-((3-chloro-1-methyl-1H-pyrazol-4-yl)amino)-8-oxo-7-phenyl-5,6,7,8-tetrahydro-9H-pyrimido[4,5-d][1,3]diazepin-9-yl)phenyl)acrylamide (Compound 29),
(E)-N-(3-(2-((3-chloro-1-methyl-1H-pyrazol-4-yl)amino)-8-oxo-7-phenyl-5,6,7,8-tetrahydro-9H-pyrimido[4,5-d][1,3]diazepin-9-yl)phenyl)but-2-enamide (Compound 30),
(E)-N-(3-(2-((3-chloro-1-methyl-1H-pyrazol-4-yl)amino)-8-oxo-7-phenyl-5,6,7,8-tetrahydro-9H-pyrimido[4,5-d][1,3]diazepin-9-yl)phenyl)-4-(dimethylamino)but-2-enamide (Compound 31),
(E)-N-(3-(2-((3-chloro-1-methyl-1H-pyrazol-4-yl)amino)-8-oxo-7-phenyl-5,6,7,8-tetrahydro-9H-pyrimido[4,5-d][1,3]diazepin-9-yl)phenyl)-4-morpholinobut-2-enamide (Compound 32),
(E)-N-(3-(2-((3-chloro-1-methyl-1H-pyrazol-4-yl)amino)-8-oxo-7-phenyl-5,6,7,8-tetrahydro-9H-pyrimido[4,5-d][1,3]diazepin-9-yl)phenyl)-4-fluorobut-2-enamide (Compound 33),
(E)-4-(dimethylamino)-N-(3-(2-((3-methyl-4-(4-methylpiperazin-1-yl)phenyl)amino)-8-oxo-7-phenyl-5,6,7,8-tetrahydro-9H-pyrimido[4,5-d][1,3]diazepin-9-yl)phenyl)but-2-enamide (Compound 34),
N-(3-(2-((3-fluoro-1-methyl-1H-pyrazol-4-yl)amino)-8-oxo-7-phenyl-5,6,7,8-tetrahydro-9H-pyrimido[4,5-d][1,3]diazepin-9-yl)phenyl)acrylamide (Compound 35),
N-(3-(2-((4-(1-methylpiperidin-4-yl)phenyl)amino)-8-oxo-7-phenyl-5,6,7,8-tetrahydro-9H-pyrimido[4,5-d][1,3]diazepin-9-yl)phenyl)acrylamide (Compound 36),
N-(3-(2-((4-(4-methylpiperazin-1-yl)phenyl)amino)-8-oxo-7-phenyl-5,6,7,8-tetrahydro-9H-pyrimido[4,5-d][1,3]diazepin-9-yl)phenyl)acrylamide (Compound 37),
(E)-4-(dimethylamino)-N-(3-(2-((4-(1-methylpiperidin-4-yl)phenyl)amino)-8-oxo-7-phenyl-5,6,7,8-tetrahydro-9H-pyrimido[4,5-d][1,3]diazepin-9-yl)phenyl)but-2-enamide (Compound 38),
N-(3-(2-((2-methoxy-4-(4-methylpiperazin-1-yl)phenyl)amino)-8-oxo-7-phenyl-5,6,7,8-tetrahydro-9H-pyrimido[4,5-d][1,3]diazepin-9-yl)phenyl)acrylamide (Compound 39),
(E)-N-(3-(2-((3-chloro-1-methyl-1H-pyrazol-4-yl)amino)-8-oxo-7-phenyl-5,6,7,8-tetrahydro-9H-pyrimido[4,5-d][1,3]diazepin-9-yl)phenyl)-4-(piperidin-1-yl)but-2-enamide (Compound 40),
N-(3-(2-((3-methyl-4-(4-methylpiperazin-1-yl)phenyl)amino)-8-oxo-7-phenyl-5,6,7,8-tetrahydro-9H-pyrimido[4,5-d][1,3]diazepin-9-yl)phenyl)acrylamide (Compound 41),
N-(3-(2-((3-chloro-1-methyl-1H-pyrazol-4-yl)amino)-7-(4-fluorophenyl)-8-oxo-5,6,7,8-tetrahydro-9H-pyrimido[4,5-d][1,3]diazepin-9-yl)phenyl)acrylamide (Compound 42),
N-(3-(2-((2-fluoropyridin-3-yl)amino)-8-oxo-7-phenyl-5,6,7,8-tetrahydro-9H-pyrimido[4,5-d][1,3]diazepin-9-yl)phenyl)acrylamide (Compound 43),
(E)-4-(dimethylamino)-N-(3-(2-((2-fluoropyridin-3-yl)amino)-8-oxo-7-phenyl-5,6,7,8-tetrahydro-9H-pyrimido[4,5-d][1,3]diazepin-9-yl)phenyl)but-2-enamide (Compound 44),
N-(3-((9-methyl-8-oxo-7-phenyl-6,7,8,9-tetrahydro-5H-pyrimido[4,5-d][1,3]diazepin-2-yl)amino)phenyl)acrylamide (Compound 45),
(E)-N-(3-(2-((3-chloro-1-methyl-1H-pyrazol-4-yl)amino)-8-oxo-7-phenyl-5,6,7,8-tetrahydro-9H-pyrimido[4,5-d][1,3]diazepin-9-yl)phenyl)-4-methoxybut-2-enamide (Compound 46).

An embodiment of the present disclosure relates to a compound of Formula I or its stereoisomers, tautomers, pharmaceutically acceptable salts, polymorphs, solvates and hydrates thereof, for treating disease associated with epidermal growth factor receptor (EGFR) family kinases.

Another embodiment of the present disclosure relates to a compound of Formula I or its stereoisomers, tautomers, pharmaceutically acceptable salts, polymorphs, solvates and hydrates thereof, for treating cancer.

Another embodiment of the present disclosure relates to a compound Formula I, or its stereoisomers, tautomers, pharmaceutically acceptable salts, polymorphs, solvates and hydrates thereof, for treating disease or condition associated with non-small cell or small cell lung cancer or prostate cancer or head and neck cancer or breast cancer or colorectal cancer.

The present disclosure relates to a pharmaceutical composition comprising a compound of Formula I or a pharmaceutically acceptable salt thereof together with a pharmaceutically acceptable carrier, optionally in combination with one or more other pharmaceutical compositions.

The present disclosure further relates to the process of preparation of compounds of Formula I or its stereoisomers, tautomers, pharmaceutically acceptable salts, polymorphs, solvates and hydrates thereof.

### Uses

Some embodiments provided herein describe a class of compounds that are useful as epidermal growth factor receptor (EGFR) family kinase inhibitors. Some embodiments provided herein describe a class of compounds that are useful as HER2 inhibitors. Some embodiments provided herein describe a class of compounds that are useful as EGFR inhibitors. Some embodiments provided herein describe a class of compounds that are useful as EGFR del19/T790M inhibitors. Some embodiments provided herein describe a class of compounds that are useful as EGFR L858R/T790M inhibitors. In some embodiments, the compounds described herein have improved potency and/or beneficial activity profiles and/or beneficial selectivity profiles and/or increased efficacy and/or improved safety profiles (such as reduced side effects) and/or improved pharmacokinetic properties. In some embodiments, the compounds described herein are selective inhibitors of EGFR del19/T790M over WT EGFR. In some embodiments, the compounds described herein are selective inhibitors of EGFR L858R/T790M over WT EGFR.

In some embodiments, the compounds described herein are useful to treat, prevent or ameliorate a disease or condition which displays drug resistance associated with EGFR del19/T790M activation. In some embodiments, the compounds described herein are useful to treat, prevent or ameliorate a disease or condition which displays drug resistance associated with EGFR L858R/T790M activation.

In some embodiments, EGFR family kinase mutants are detected with a commercially available test kit. In some embodiments, EGFR family kinase mutants are detected with a reverse transcription polymerase chain reaction (RT-PCR)-based method. In some embodiments, EGFR family kinase mutants are detected with a sequencing-based method. In some embodiments, EGFR family kinase mutants are detected with a mass spectrometry genotyping-based method. In some embodiments, EGFR family kinase mutants are detected with an immunohistochemistry-based method. In some embodiments, EGFR family kinase mutants are detected with a molecular diagnostics panel. In some embodiments, EGFR family kinase mutants are detected from a tumor sample. In some embodiments, EGFR family kinase mutants are detected from circulating DNA. In some embodiments, EGFR family kinase mutants are detected from tumor cells.

In one aspect, provided herein is a method of inhibiting an epidermal growth factor receptor (EGFR) family kinase mutant in a subject in need thereof, comprising administering to the subject a therapeutically effective amount of a compound of Formula I, or a pharmaceutically acceptable salt thereof.

In another aspect, provided herein is a method of inhibiting a human epidermal growth factor receptor 2 (HER2) mutant in a subject in need thereof, comprising administering to the subject a therapeutically effective amount of a compound of Formula I, or a pharmaceutically acceptable salt thereof. In some embodiments, the HER2 mutant comprises an insertion in exon 20, an in-frame deletion and insertion in exon 20, a substitution in the extracellular domain, an extracellular truncation, or a substitution in exon 30. In some embodiments, the HER2 mutant is A775ins_G776insYVMA, A775_G776insSVMA, A775_G776insVVMA, G776del insVC, G776del insLC, G776del insAV, G776del insAVGC, S310F, S310Y, p95, V842I, or P780_Y781insGSP. In some embodiments, the HER2 mutant is A775ins_G776insYVMA. In some embodiments, the HER2 mutant is A775_G776insSVMA. In some embodiments, the HER2 mutant is A775_G776insVVMA. In some embodiments, the HER2 mutant is G776del insVC. In some embodiments, the HER2 mutant is G776del insLC. In some embodiments, the HER2 mutant is G776del insAV. In some embodiments, the HER2 mutant is G776del insAVGC. In some embodiments, the HER2 mutant is S310F. In some embodiments, the HER2 mutant is S310Y. In some embodiments, the HER2 mutant is p95. In some embodiments, the HER2 mutant is V842I. In some embodiments, the HER2 mutant is P780_Y781insGSP.

In another aspect, provided herein is a method of inhibiting an epidermal growth factor receptor (EGFR) mutant in a subject in need thereof, comprising administering to the subject a therapeutically effective amount of a compound of Formula I, or a pharmaceutically acceptable salt thereof.

In another aspect, provided herein is a method of inhibiting a drug-resistant epidermal growth factor receptor (EGFR) mutant in a subject in need thereof, comprising administering to the subject a therapeutically effective amount of a compound of Formula I, or a pharmaceutically acceptable salt thereof. In some embodiments, the drug-resistant EGFR mutant is del19/T790M EGFR or L858R/T790M EGFR.

In another aspect, provided herein is a method of inhibiting human epidermal growth factor receptor 2 (HER2) in a subject in need thereof, comprising administering to the subject a therapeutically effective amount of a compound of Formula I, or a pharmaceutically acceptable salt thereof, wherein the compound exhibits greater inhibition of a HER2 mutant relative to wild-type EGFR. In some embodiments, the HER2 mutant comprises an insertion in exon 20, an in-frame deletion and insertion in exon 20, a substitution in the extracellular domain, an extracellular truncation, or a substitution in exon 30. In some embodiments, the HER2 mutant is A775ins_G776insYVMA, A775_G776insSVMA, A775_G776insVVMA, G776del insVC, G776del insLC, G776del insAV, G776del insAVGC, S310F, S310Y, p95, V842I, or P780_Y781insGSP. In some embodiments, the HER2 mutant is A775ins_G776insYVMA. In some embodiments, the HER2 mutant is A775_G776insSVMA. In some embodiments, the HER2 mutant is A775_G776insVVMA. In some embodiments, the HER2 mutant is G776del insVC. In some embodiments, the HER2 mutant is G776del insLC. In some embodiments, the HER2 mutant is G776del insAV. In some embodiments, the HER2 mutant is G776del insAVGC. In some embodiments, the HER2 mutant is S310F. In some embodiments, the HER2 mutant is S310Y. In some embodiments, the HER2 mutant is p95. In some embodiments, the HER2 mutant is V842I. In some embodiments, the HER2 mutant is P780_Y781insGSP.

In another aspect, provided herein is a method of inhibiting epidermal growth factor receptor (EGFR) in a subject in need thereof, comprising administering to the subject a therapeutically effective amount of a compound of Formula I, or a pharmaceutically acceptable salt thereof, wherein the compound exhibits greater inhibition of an EGFR mutant relative to wild-type EGFR.

In some embodiments, the EGFR mutant comprises a substitution in exon 18, a deletion in exon 19, a substitution in exon 20, an insertion in exon 20, a mutation in the extracellular domain, or a substitution in exon 21. In some embodiments, the EGFR mutant is del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, D770_N771insSVD EGFR, H773insNPH EGFR, A767_dupASV EGFR, or 773insAH EGFR. In some embodiments, the EGFR mutant is del19/T790M EGFR or L858R/T790M EGFR. In some embodiments, the EGFR mutant is del19/T790M EGFR. In some embodiments, the EGFR mutant is L858R/T790M EGFR.

In another aspect, provided herein is a method of treating a disease or disorder associated with epidermal growth factor receptor (EGFR) in a subject in need thereof, comprising administering to the subject a therapeutically effective amount of a compound of Formula I, or a pharmaceutically acceptable salt thereof.

In some embodiments, the disease or disorder in the subject comprises a HER2 mutation. In some embodiments, the HER2 mutation comprises an insertion in exon 20, an in-frame deletion and insertion in exon 20, a substitution in the extracellular domain, an extracellular truncation, or a substitution in exon 30. In some embodiments, the HER2 mutation is A775ins_G776insYVMA, A775_G776insSVMA, A775_G776insVVMA, G776del insVC, G776del insLC, G776del insAV, G776del insAVGC, S310F, S310Y, p95, V842I, or P780_Y781insGSP. In some embodiments, the HER2 mutation is A775ins_G776insYVMA. In some embodiments, the HER2 mutation is A775_G776insSVMA. In some embodiments, the HER2 mutation is A775_G776insVVMA. In some embodiments, the HER2 mutation is G776del insVC. In some embodiments, the HER2 mutation is G776del insLC. In some embodiments, the HER2 mutation is G776del insAV. In some embodiments, the HER2 mutation is G776del insAVGC. In some embodiments, the HER2 mutation is S310F. In some embodiments, the HER2 mutation is S310Y. In some embodiments, the HER2 mutation is p95. In some embodiments, the HER2 mutation is V842I. In some embodiments, the HER2 mutation is P780_Y781insGSP.

In some embodiments, the disease or disorder in the subject comprises an EGFR mutation. In some embodiments, the EGFR mutation comprises a substitution in exon 18, a deletion in exon 19, a substitution in exon 20, an insertion in exon 20, a mutation in the extracellular domain, or a substitution in exon 21. In some embodiments, the EGFR mutation is del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, D770_N771insSVD EGFR, H773insNPH EGFR, A767_dupASV EGFR, or 773insAH EGFR. In some embodiments, the EGFR mutation is del19/T790M EGFR or L858R/T790M EGFR. In some embodiments, the EGFR mutation is del19/T790M EGFR. In some embodiments, the EGFR mutation is L858R/T790M EGFR.

In another aspect, provided herein is a method of treating cancer in a subject in need thereof, comprising administering to the subject a therapeutically effective amount of a compound of Formula I, or a pharmaceutically acceptable salt thereof. In some embodiments, the cancer displays drug resistance associated with EGFR del19/T790M activation. In some embodiments, the cancer displays drug resistance associated with EGFR L858R/T790M activation. Other embodiments provided herein describe the use of the compounds described herein for treating cancer.

In some embodiments, the cancer is selected from bladder cancer, prostate cancer, breast cancer, cervical cancer, colorectal cancer, endometrial cancer, gastric cancer, glioblastoma, head and neck cancer, lung cancer, and non-small cell lung cancer. In some embodiments, the cancer is selected from non-small cell lung cancer, prostate cancer, head and neck cancer, breast cancer, colorectal cancer, and glioblastoma. In some embodiments, the cancer is non-small cell lung cancer. In some embodiments, the cancer is prostate cancer. In some embodiments, the cancer is head and neck cancer. In some embodiments, the cancer is breast cancer. In some embodiments, the cancer is colorectal cancer. In some embodiments, the cancer is glioblastoma.

In some embodiments, the cancer in the subject comprises a HER2 mutation. In some embodiments, the HER2 mutation comprises an insertion in exon 20, an in-frame deletion and insertion in exon 20, a substitution in the extracellular domain, an extracellular truncation, or a substitution in exon 30. In some embodiments, the HER2 mutation is A775ins_G776insYVMA, A775_G776insSVMA, A775_G776insVVMA, G776del insVC, G776del insLC, G776del insAV, G776del insAVGC, S310F, S310Y, p95, V842I, or P780_Y781insGSP. In some embodiments, the HER2 mutation is A775ins_G776insYVMA. In some embodiments, the HER2 mutation is A775_G776insSVMA. In some embodiments, the HER2 mutation is A775_G776insVVMA. In some embodiments, the HER2 mutation is G776del insVC. In some embodiments, the HER2 mutation is G776del insLC. In some embodiments, the HER2 mutation is G776del insAV. In some embodiments, the HER2 mutation is G776del insAVGC. In some embodiments, the HER2 mutation is S310F. In some embodiments, the HER2 mutation is S310Y. In some embodiments, the HER2 mutation is p95. In some embodiments, the HER2 mutation is V842I. In some embodiments, the HER2 mutation is P780_Y781insGSP.

In some embodiments, the cancer in the subject comprises an EGFR mutation. In some embodiments, the EGFR mutation comprises a substitution in exon 18, a deletion in exon 19, a substitution in exon 20, an insertion in exon 20, a mutation in the extracellular domain, or a substitution in exon 21. In some embodiments, the EGFR mutation is del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, D770_N771insSVD EGFR, H773insNPH EGFR, A767_dupASV EGFR, or 773insAH EGFR. In some embodiments, the EGFR mutation is del19/T790M EGFR or L858R/T790M EGFR. In some embodiments, the EGFR mutation is del19/T790M EGFR. In some embodiments, the EGFR mutation is L858R/T790M EGFR.

In another aspect, provided herein is a method of treating inflammatory disease in a subject in need thereof, comprising administering to the subject a therapeutically effective amount of a compound of Formula I, or a pharmaceutically acceptable salt thereof. Also described herein is the use of the compounds described herein for treating inflammatory diseases associated with EGFR del19/T790M activation. Also described herein is the use of the compounds described herein for treating inflammatory diseases associated with EGFR L858R/T790M activation.

In some embodiments, the inflammatory disease is selected from psoriasis, eczema, and atherosclerosis. In some embodiments, the inflammatory disease is psoriasis. In some embodiments, the inflammatory disease is eczema. In some embodiments, the inflammatory disease is atherosclerosis.

In some embodiments, the inflammatory disease in the subject comprises a HER2 mutation. In some embodiments, the HER2 mutation comprises an insertion in exon 20, an in-frame deletion and insertion in exon 20, a substitution in the extracellular domain, an extracellular truncation, or a substitution in exon 30. In some embodiments, the HER2 mutation is A775ins_G776insYVMA, A775_G776insSVMA, A775_G776insVVMA, G776del insVC, G776del insLC, G776del insAV, G776del insAVGC, S310F, S310Y, p95, V842I, or P780_Y781insGSP. In some embodiments, the HER2 mutation is A775ins_G776insYVMA. In some embodiments, the HER2 mutation is A775_G776insSVMA. In some embodiments, the HER2 mutation is A775_G776insVVMA. In some embodiments, the HER2 mutation is G776del insVC. In some embodiments, the HER2 mutation is G776del insLC. In some embodiments, the HER2 mutation is G776del insAV. In some embodiments, the HER2 mutation is G776del insAVGC. In some embodiments, the HER2 mutation is S310F. In some embodiments, the HER2 mutation is S310Y. In some embodiments, the HER2 mutation is p95. In some embodiments, the HER2 mutation is V842I. In some embodiments, the HER2 mutation is P780_Y781insGSP.

In some embodiments, the inflammatory disease in the subject comprises an EGFR mutation. In some embodiments, the EGFR mutation comprises a substitution in exon 18, a deletion in exon 19, a substitution in exon 20, an insertion in exon 20, a mutation in the extracellular domain, or a substitution in exon 21. In some embodiments, the EGFR mutation is del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, D770_N771insSVD EGFR, H773insNPH EGFR, A767_dupASV EGFR, or 773insAH EGFR. In some embodiments, the EGFR mutation is del19/T790M EGFR or L858R/T790M EGFR. In some embodiments, the EGFR mutation is del19/T790M EGFR. In some embodiments, the EGFR mutation is L858R/T790M EGFR.

### Administration and Pharmaceutical Composition

In certain embodiments, the EGFR inhibitory compound as described herein is administered as a pure chemical. In other embodiments, the EGFR inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21st Ed. Mack Pub. Co., Easton, PA (2005)).

Provided herein is a pharmaceutical composition comprising at least one EGFR inhibitory compound as described herein, or a stereoisomer, pharmaceutically acceptable salt, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject or patient) of the composition.

One embodiment provides a pharmaceutical composition comprising a compound disclosed herein, or a pharmaceutically acceptable salt, solvate, or prodrug thereof, and a pharmaceutically acceptable excipient.

In certain embodiments, the EGFR inhibitory compound disclosed herein is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21st Ed. Mack Pub. Co., Easton, PA (2005)).

The dose of the composition comprising at least one EGFR inhibitory compound as described herein differ, depending upon the patient's condition, that is, stage of the disease, general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome), or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### EXAMPLES

### Example 1: Synthetic Procedures

Yields reported herein refer to purified products (unless specified) and are not optimised. Analytical TLC was performed on Merck silica gel 60 F₂₅₄ aluminum-backed plates. Compounds were visualised by UV light and/or stained either with iodine, potassium permanganate or ninhydrin solution. Flash column chromatography was performed on silica gel (100-200 M) or flash chromatography. ¹H-NMR spectra were recorded on a Bruker Avance-400 MHz spectrometer with a BBO (Broad Band Observe) and BBFO (Broad Band Fluorine Observe) probe. Chemical shifts (δ) are expressed in parts per million (ppm) downfield by reference to tetramethylsilane (TMS) as the internal standard. Splitting patterns are designated as s (singlet), d (doublet), t (triplet), q (quartet), m (multiplet) and bs (broad singlet). Coupling constants (J) are given in hertz (Hz). LC-MS analyses were performed on either an Acquity BEH C-18 column (2.10×100 mm, 1.70 μm) or on a Acquity HSS-T3 column (2.10×100 mm, 1.80 μm) using the Electrospray Ionisation (ESI) technique.

The following solvents, reagents or scientific terminology may be referred to by their abbreviations:
TLC Thin Layer Chromatography
DCM Dichloromethane
THF Tetrahydrofuran
MeOH Methanol
EtOH Ethanol
IPA Isopropyl alcohol
EtOAc Ethyl acetate
Et₂O Diethyl ether
DMA N,N-Dimethylacetamide
DMF N,N-Dimethylformamide
TEA/Et₃N Triethylamine
DMSO Dimethylsulfoxide
DIPEA Diisopropylethylamine (Hunig's base)
MeI Methyliodide
NBS N-Bromosuccinimide
TBAB Tetrabutylammonium bromide
TBAI Tetrabutylammonium iodide
DIBAL-H Diisobutylaluminum hydride
TFA Trifluoroacetic acid
AcOH Acetic acid
Boc tert-butoxycarbonyl
Cat Catalytic
mL milliliters
mmol millimoles
h hour or hours
min minute or minutes
g grams
mg milligrams
μl Microlitres
eq Equivalents
rt or RT Room temperature, ambient, about 27° C.
MS Mass spectrometry
Boc tert-Butyloxycarbonyl
m-CPBA meta-Chloroperbenzoic acid
T3P Propane phosphonic acid anhydride
BH₃-DMS Borane dimethylsulfide complex
LiBH₄ Lithium aluminum hydride
NaBH₄ Sodium borohydride
H₂ Hydrogen
Pd/C Palladium on charcoal
1,2-DCE 1,2-Dichloroethane

### General Procedure A

To an ice-cold solution of chloro derivatives (1.0 eq) in isopropanol (10 volume) were added respective amines (1.2 eq) and trifluoroacetic acid (2.0 eq). Then the reaction mixture was heated at 100° C. for 16 hours. After completion of the reaction (TLC monitoring), the solvent was concentrated under reduced pressure, followed by saturated solution of sodium bicarbonate was added and extracted with dichloromethane (3 times). The combined organic layers were washed with brine solution, dried over anhydrous sodium sulfate and evaporated under reduced pressure afforded the crude product. The crude was triturated with diethyl ether afforded the desired products which was used directly for the next step without any further purification.

### General Procedure B

An ice-cold solution of products (1.0 eq) obtained from General Procedure A in 20% trifluoroacetic acid in dichloromethane (10 volume) was stirred at room temperature for 3-16 hours. After completion of the reaction (TLC monitoring), the solvent was evaporated to dryness. The reaction mixture diluted with saturated solution of sodium bicarbonate and extracted with 5% methanol in dichloromethane (3 times). The combined organic layers were washed with brine solution, dried over sodium sulfate and evaporated under reduced pressure. The crude was triturated with diethyl ether or purified over combiflash, eluted with 5-10% methanol in dichloromethane afforded the desired products.

### General Procedure C

To an ice-cold solution of products (1.0 eq) obtained from General Procedure B in dichloromethane (10 volume) was added triethylamine (5 eq), respective acids (1.1 eq), and propylphosphonic anhydride (T₃P, 50% in ethyl acetate, 2.5 eq). Then the reaction mixture was stirred at room temperature for 16 hours. After completion of reaction (TLC monitoring), reaction mixture was diluted with saturated solution of sodium bicarbonate and extracted with 5% methanol in dichloromethane. The combined organic layers were washed with brine, dried over anhydrous sodium sulfate, filtered and concentrated under reduced pressure afforded the crude product. The crude was purified over combiflash or Prep-TLC or Prep-HPLC afforded the final compounds.

### General Procedure C₁

To an ice-cold solution of products (1.0 eq) obtained from General Procedure B in acetonitrile (10 volume) was added N,N-diisopropylethylamine (5 eq), respective acids (1.1 eq), and HATU (2.5 eq). Then the reaction mixture heated at 70° C. for 16 hours. After completion of reaction (TLC monitoring), reaction mixture was diluted with saturated solution of sodium bicarbonate and extracted with 5% methanol in dichloromethane. The combined organic layers were washed with brine, dried over anhydrous sodium sulfate, filtered and concentrated under reduced pressure afforded the crude product. The crude was purified over combiflash or Prep-TLC or Prep-HPLC afforded the final compounds.

### General Procedure D

To a solution of products (1.0 eq) obtained from General Procedure B in dichloromethane or dichloromethane:tetrahydrofuran (1:1) (10 volume) at −30° C. were added triethylamine (5 eq) and acryloyl chloride (1.0 eq). Then the reaction mixture was stirred at the same temperature for 30 minutes to 2 hours. After completion of reaction (monitored by TLC), water was added and extracted with dichloromethane (3 times). The combined organic layers were washed with brine, dried over anhydrous sodium sulphate and concentrated under reduced pressure. The crudes were purified by Prep-HPLC afforded the final compounds.

### General Procedure E

To a solution of products (1.0 eq) obtained from General Procedure B in tetrahydrofuran:water (3:1) (10 volume) at 0° C. was added triethylamine (3 to 5 eq) and acryloyl chloride (1.5 eq). The mixture was stirred at the same temperature for 2 hours. After completion of reaction (monitored by TLC), water was diluted with water and extracted with ethyl acetate (3 times). The combined organic layers were washed with brine, dried over anhydrous sodium sulphate and concentrated under reduced pressure. The crudes were purified by Prep-HPLC afforded the final compounds

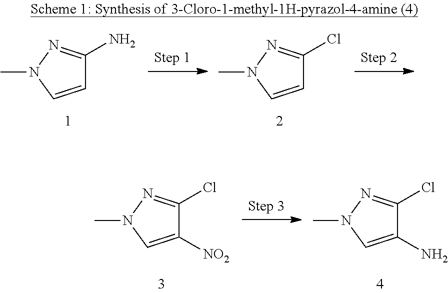

### Step 1: Synthesis of 3-Chloro-1-methyl-1H-pyrazole (2)

To an ice cold solution of 1-methyl-1H-pyrazol-3-amine (1) (50 g, 0.52 mol) in acetonitrile (400 mL) was added copper(I) chloride (154 g, 1.56 mol). The resulting mixture was stirred at room temperature for 30 minutes, followed by tert-butyl nitrite (268 g, 2.60 mol) was added and stirred at 60° C. for 30 minutes. After completion of the reaction (TLC monitoring), the reaction mixture was poured into water and extracted with ethyl acetate (3×300 mL). The combined organic layers were washed with brine and dried over anhydrous sodium sulfate and concentrated under reduced pressure to afford the desired product (2) (32.0 g; Yield: 53%). ¹H-NMR (400 MHz, CDCl₃): δ 7.27 (d, J=2.4 Hz, 1H), 6.15 (d, J=2.4 Hz, 1H), 3.85 (s, 3H).

### Step 2: Synthesis of 3-Chloro-1-methyl-4-nitro-1H-pyrazole (3)

To an ice-cold solution of 3-chloro-1-methyl-1H-pyrazole (2) (30 g, 0.26 mol) in concentrated sulphuric acid (50 mL) was slowly added fuming nitric acid (40 mL, 0.91 mol) drop wise. The resulting reaction mixture was stirred at room temperature for 6 hours. After completion of reaction (TLC monitoring), the reaction mixture was poured into ice-cold water, the resulted solid was filtered and washed with pentane to afford the desired product (3) as yellow solid (30 g; Yield: 73%). ¹H NMR (400 MHz, CDCl₃): δ 8.16 (s, 1H), 3.94 (s, 3H).

### Step 3: Synthesis of 3-Chloro-1-methyl-1H-pyrazol-4-amine (4)

To a solution of 3-chloro-1-methyl-4-nitro-1H-pyrazole (3) (30 g, 0.186 mol) in methanol (300 mL) was added raney nickel (3 g, 10% w/w). The reaction was stirred under hydrogen atmosphere for 16 hours. The reaction was monitored by TLC (after completion), the reaction mixture was filtered through celite bed and washed with methanol. The filtrate was concentrated under reduced pressure to afford the desired product (4) as viscous liquid (14.0 g; Yield: 57%). ¹H NMR (400 MHz, DMSO-d₆): δ 7.09 (s, 1H), 3.88 (s, 2H), 3.64 (s, 3H).

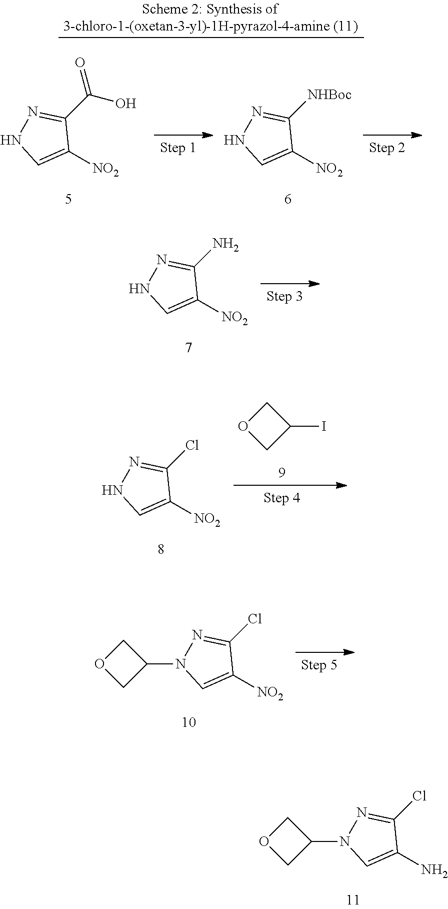

### Step 1: Synthesis of tert-butyl (4-nitro-1H-pyrazol-3-yl)carbamate (6)

To an ice-cold solution of 4-nitro-1H-pyrazole-3-carboxylic acid (5) (2.5 g, 15.9 mmol) in toluene (50 mL) was added triethylamine (5.6 mL, 39.75 mmol) and diphenyl phosphoryl azide (4.25 g, 17.5 mmol). The reaction mixture was stirred at room temperature for 6 hours. After completion of reaction (TLC monitoring), tert-butanol (25 mL) was added and heated at 130° C. for 16 hours. After completion of reaction, the reaction mixture was cooled to 0° C., quenched with water (100 mL) and extracted with ethyl acetate (3×100 mL). The combined organic layer was washed with brine, dried over anhydrous sodium sulfate, filtered and concentrated under reduced pressure. The crude was purified using combiflash and was eluted with 30% ethyl acetate in heptane to afford the desired product (6) (0.6 g, Yield: 16%). ¹H NMR (400 MHz, DMSO-d₆): δ 13.58 (bs, 1H), 9.33 (s, 1H), 8.44 (s, 1H), 1.44 (s, 9H).

### Step 2: Synthesis of 4-nitro-1H-pyrazol-3-amine (7)

An ice-cold solution of tert-butyl (4-nitro-1H-pyrazol-3-yl)carbamate (6) (0.5 g, 21.9 mmol) in hydrochloric acid in dioxane (5 mL, 4M) was stirred at room temperature for 6 hours. After completion of reaction (TLC monitoring), solvent was evaporated under reduced pressure to get desired product (7) (0.287 g, Yield: 80%) as off white solid. LCMS [M+H]⁺ 129.10.

### Step 3: Synthesis of 3-chloro-4-nitro-1H-pyrazole (8)

To an ice-cold solution of 4-nitro-1H-pyrazol-3-amine (7) (0.5 g, 3.9 mmol) in hydrochloric acid (5.0 mL) was added aqueous solution of sodium nitrite (0.547 g, 7.8 mmol) in water (1.0 mL). The resulting reaction mixture stirred at same temperature for 1 hour, followed by addition of copper(I) chloride (0.773 g, 7.8 mmol) and stirred at room temperature for 16 hours. After completion of reaction (TLC monitoring), reaction mixture was diluted with ice-cold water (50 mL) and extracted with dichloromethane (3×50 mL). The combined organic layer was dried over anhydrous sodium sulfate, filtered and concentrated under reduced pressure to afford the desired product (8) (0.25 g, Yield: 43%). LCMS [M−H]⁻ 146.10.

### Step 4: Synthesis of 3-chloro-4-nitro-1-(oxetan-3-yl)-1H-pyrazole (10)

To a stirred solution of 3-chloro-4-nitro-1H-pyrazole (8) (0.5 g, 3.39 mmol) in N,N-dimethylformamide (7.0 mL) were added 3-iodooxetane (9) (0.448 mL, 5.08 mmol), and cesium carbonate (2.21 g, 6.78 mmol). Then the reaction mixture was irradiated in microwave at 140° C. for 1 hour. After completion of reaction (TLC), the reaction mixture was cooled to room temperature, diluted with water (20 mL) and extracted with ethyl acetate (25 mL×3). The combined organic layer was washed with water (25 mL×2), brine (25 mL), dried over anhydrous sodium sulfate and evaporated under reduced pressure. The crude product was purified by silica gel flash column chromatography using 35% ethyl acetate in hexane as the eluent to afford 3-chloro-4-nitro-1-(oxetan-3-yl)-1H-pyrazole (10) (0.35 g, 1.72 mmol) as yellow solid. ¹H NMR (400 MHz, DMSO-d6): δ 9.11 (s, 1H), 5.71-5.55 (m, 1H), 4.91-4.82 (m, 4H)

### Step 5: Synthesis of 3-chloro-1-(oxetan-3-yl)-1H-pyrazol-4-amine (11)

To a stirred solution of 3-chloro-4-nitro-1-(oxetan-3-yl)-1H-pyrazole (10) (0.750 g, 3.68 mmol) in ethyl acetate (8.00 mL) was added platinum oxide (0.077 g, 0.368 mmol) under the hydrogen atmosphere and the reaction mixture was stirred at room temperature for 1 hour. Progress of the reaction was monitored by TLC, then the reaction mixture was filtered through the celite bed and the filtrate was concentrated under reduced pressure. The crude product was triturated with hexane and dried to afford 3-chloro-1-(oxetan-3-yl)-1H-pyrazol-4-amine (11) (0.6 g, 3.46 mmol) as purple solid. LCMS [M+H]⁺ 174.

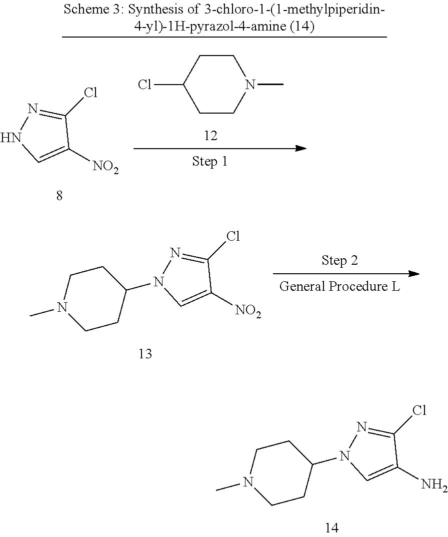

### Step 1: Synthesis of 4-(3-chloro-4-nitro-1H-pyrazol-1-yl)-1-methylpiperidine (13)

To a solution of 3-chloro-4-nitro-1H-pyrazole (8) (8.0 g, 0.054 mol) in N,N-dimethylformamide (100 mL) were added cesium carbonate (35.44 g, 0.108 mol) and 4-chloro-1-methylpiperidine (12) (10.90 g, 0.081 mol) and the reaction mixture was heated at 120° C. for 16 hours. After completion of reaction (TLC monitoring), reaction mixture was diluted with ice-cold water and extracted with 10% methanol in dichloromethane (3×100 mL). The combined organic layer was washed with brine (50 mL), dried over anhydrous sodium sulfate, filtered and concentrated under reduced pressure. The crude product was purified by combiflash chromatography using 10% methanol in dichloromethane as an eluent to afford 4-(3-chloro-4-nitro-1H-pyrazol-1-yl)-1-methylpiperidine (13) as brown solid (5.0 g, Yield: 38%). ¹H NMR (400 MHz, DMSO-d₆): δ 9.05 (s, 1H), 4.25-4.18 (m, 1H), 2.90-2.80 (m, 2H), 2.21 (s, 3H), 2.10-1.85 (m, 6H).

### Step 2: Synthesis of 3-chloro-1-(1-methylpiperidin-4-yl)-1H-pyrazol-4-amine (14)

To a stirred solution of 4-(3-chloro-4-nitro-1H-pyrazol-1-yl)-1-methylpiperidine (13) (0.8 g, 3.27 mmol) in ethanol (12.0 mL), water (4.00 mL) were added iron (1.83 g, 32.7 mmol), and ammonium chloride (1.75 g, 32.7 mmol). Then the reaction mixture was heated at 90° C. for 2 hours. After completion of reaction (TLC monitoring), the reaction mixture was cooled to room temperature, filtered through celite and the filtrate was evaporated. The residue was diluted with water (25 mL) and extracted with 10% methanol in dichloromethane (3×50 mL). The combined organic layer was washed with brine (25 mL), dried over anhydrous sodium sulphate, filtered and concentrated under reduced pressure. The crude product was purified using combiflash purifier and was eluted with 4% methanol in dichloromethane to get 3-chloro-1-(1-methylpiperidin-4-yl)-1H-pyrazol-4-amine (14) (0.25 g, 0.932 mmol) as purple gum. ¹H NMR (400 MHz, DMSO-d₆): δ 7.17 (s, 1H), 4.16 (m, 1H), 3.91-3.85 (m, 2H), 2.15 (s, 3H), 1.89-1.80 (m, 4H), 2.10-1.85 (m, 4H). LCMS [M+H]⁺ 215.21

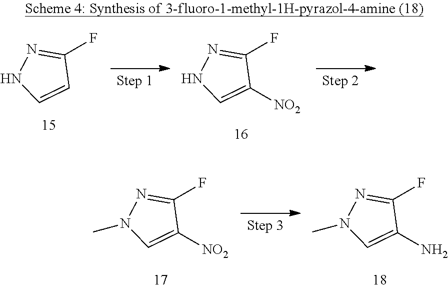

### Step 1: Synthesis of 3-fluoro-4-nitro-1H-pyrazole (16)

To a solution of 3-fluoro-1H-pyrazole (15) (2.50 g, 29.0 mmol) in sulfuric acid (4.50 mL) was added nitric acid (4.24 mL, 102 mmol) at 0° C. and the reaction mixture was heated at 70° C. for 16 hours. After completion of starting material (as monitor by TLC), the reaction mixture was poured into ice-cold water and was extracted with ethyl acetate (3×50 mL). The combined organic layer was dried over sodium sulfate and concentrated under reduced pressure to afford 3-fluoro-4-nitro-1H-pyrazole (16) (3.10 g, 75%) as yellow solid. ¹H NMR (400 MHz, DMSO-d₆): δ 8.85 (s, 1H).

### Step 2: Synthesis of 3-fluoro-1-methyl-4-nitro-1H-pyrazole (17)

To a stirred solution of 3-fluoro-4-nitro-1H-pyrazole (16) (3.10 g, 23.7 mmol) and potassium carbonate (8.17 g, 59.1 mmol) in N,N-dimethylformamide (30.0 mL) was added iodomethane (3.68 mL, 59.1 mmol) drop wise at 0° C. and the stirring was continued at room temperature for 15 hours. After completion of reaction (as monitor by TLC), ice-cold water was added and extracted with ethyl acetate (3×50 mL). The combined the organic layer was washed with brine (50 mL), dried over anhydrous sodium sulphate and concentrated under reduced pressure. The crude product was purified by silica-gel column chromatography and eluted with 25% ethyl acetate in hexane to afford 3-fluoro-1-methyl-4-nitro-1H-pyrazole (17) (2.80 g, 81%) as yellow liquid. ¹H NMR (400 MHz, DMSO-d₆): δ 8.82 (s, 1H), 3.81 (s, 3H).

### Step 3: Synthesis of 3-fluoro-1-methyl-1H-pyrazol-4-amine (18)

To a stirred solution of 3-fluoro-1-methyl-4-nitro-1H-pyrazole (17) (2.80 g, 19.3 mmol) in ethyl acetate (30.0 mL) was added palladium on carbon (0.28 g, 10% w/w, 50% wet) and the reaction mixture was stirred at room temperature under hydrogen atmosphere for 24 hours. After completion of reaction (as per TLC monitoring), the reaction mixture was filtered through celite and the filtrate was concentrated under reduced pressure to afford 3-fluoro-1-methyl-1H-pyrazol-4-amine (18) (1.50 g, crude) as a black gel. ¹H NMR (400 MHz, DMSO-d₆): δ 7.01 (s, 1H), 3.68 (bs, 2H), 3.54 (s, 3H).

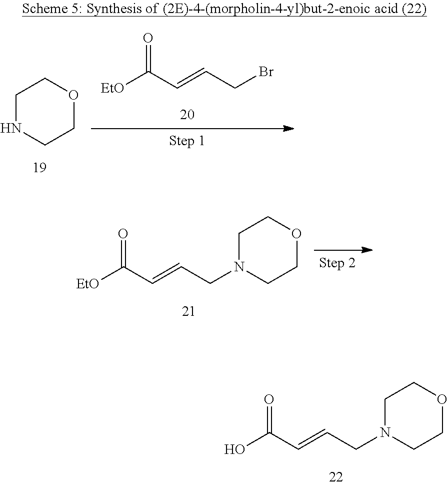

### Step 1: Synthesis of ethyl (2E)-4-(morpholin-4-yl)but-2-enoate (21)

To a stirred solution of morpholine (19) (0.5 g, 5.74 mmol) in dichloromethane (10.0 mL) were added N,N-diisopropylethylamine (1.50 mL, 8.61 mmol), ethyl (2E)-4-bromobut-2-enoate (20) (1.22 g, 6.31 mmol) and stirred at room temperature for 16 hours. The progress of the reaction was monitored by LCMS. After completion of reaction, the reaction mixture was diluted with water and extracted with dichloromethane (50 mL×2). The combined organic layer was washed with brine (25 mL), dried over sodium sulphate, filtered, and concentrated under reduced pressure to afford the desired product (21) (1.00 g, crude). LCMS [M+H]⁺ 200.1

### Step 2: Synthesis of (2E)-4-(morpholin-4-yl)but-2-enoic acid (22)

To a stirred solution of ethyl (2E)-4-(morpholin-4-yl)but-2-enoate (21) (1.00 g, 5.02 mmol) in 1,4-dioxane (10.0 mL), was added hydrochloric acid (10.0 mL, 2N aqueous) and refluxed for 3 hours. The progress of the reaction was monitored by LCMS. After completion of the reaction, reaction mixture was diluted with water (10 mL) and extracted with ethyl acetate (3×50 mL). The aqueous layer was concentrated under reduced pressure. The crude product was triturated with ethyl acetate to afford title compound (22) (0.9 g, 90%) as light brown solid. LCMS [M+H]⁺ 172.1.

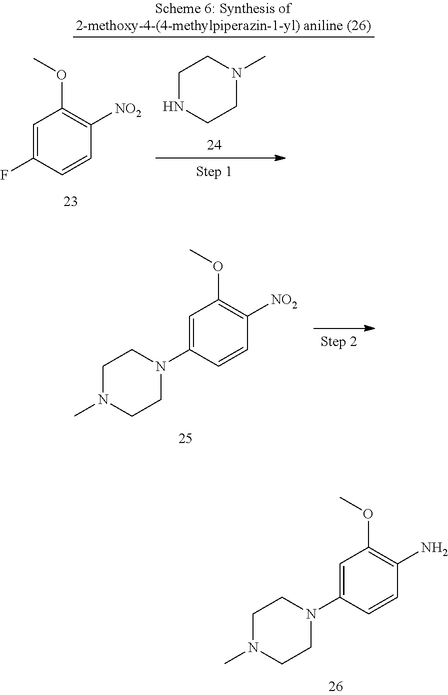

### Step 1: Synthesis of 1-(3-methoxy-4-nitrophenyl)-4-methylpiperazine (25)

To a stirred solution of 4-fluoro-2-methoxy-1-nitrobenzene (23) (5.00 g, 29.2 mmol) in N, N-dimethylformamide (50.0 mL) were added dipotassium carbonate (12.1 g, 87.7 mmol) and 1-methylpiperazine (24) (2.93 g, 29.2 mmol) at room temperature. The reaction mixture was heated at 110° C. for 16 hours. After completion of reaction, the reaction mixture was cooled and diluted with water (100 mL). The precipitated solid was filtered, dried to afford 1-(3-methoxy-4-nitrophenyl)-4-methylpiperazine (25) (7.00 g, 95%) as yellow solid. LCMS [M+H]⁺ 252.2

### Step 2: Synthesis of 2-methoxy-4-(4-methylpiperazin-1-yl) aniline (26)

To a stirred solution of 1-(3-methoxy-4-nitrophenyl)-4-methylpiperazine (25) (7.00 g, 27.9 mmol) in methanol (50.0 mL), tetrahydrofuran (50.0 mL) and water were added zinc (14.6 g, 223 mmol), ammonium chloride (11.9 g, 223 mmol) and the reaction mixture was stirred at room temperature for 2 hours. After 2 hours, (TLC and LC-MS monitoring) upon completion of reaction, the reaction mixture was filtered through celite pad and filtrate was concentrated to afford 2-methoxy-4-(4-methylpiperazin-1-yl)aniline (26) (4.50 g, 73%). LCMS [M+H]⁺ 222.2

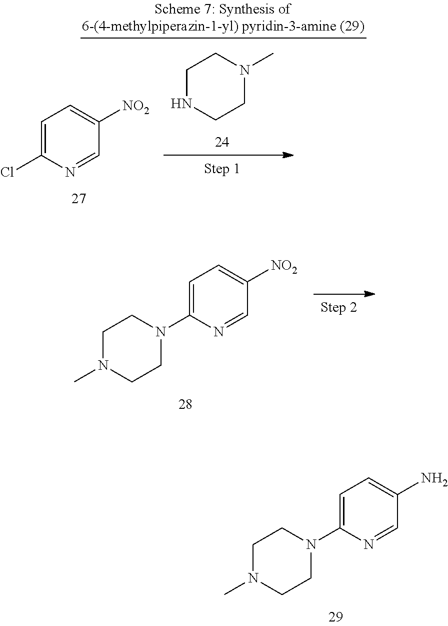

### Step 1: Synthesis of 1-methyl-4-(5-nitropyridin-2-yl) piperazine (28)

To a stirred solution of 2-chloro-5-nitropyridine (27) (5.00 g, 31.5 mmol) in N,N-dimethylformamide (50.0 mL) were added potassium carbonate (13.1 g, 94.6 mmol) and 1-methylpiperazine (24) (3.16 g, 31.5 mmol) at room temperature. The reaction mixture was heated at 110° C. for 16 hours. After completion of reaction, water was added to the reaction mixture and the precipitated solid was filtered, dried to afford 1-methyl-4-(5-nitropyridin-2-yl) piperazine (29) (6.00 g, 85%) as brown solid. LCMS [M+H]⁺ 223.1

### Step 2: Synthesis of 6-(4-methylpiperazin-1-yl) pyridin-3-amine. (29)

To a stirred solution of 1-methyl-4-(5-nitropyridin-2-yl) piperazine (28) (5.80 g, 26.1 mmol) in methanol (30.0 mL), tetrahydrofuran (30.0 mL) was added palladium on carbon (1.0 g, 10% w/w) and the reaction mixture was stirred at room temperature under hydrogen atmosphere (using bladder) for 16 hours. The progress of the reaction was monitored by LC-MS and TLC. The resulting reaction mixture was filtered through the celite and the filtrate was evaporated under vacuum to get 6-(4-methylpiperazin-1-yl) pyridin-3-amine (29) (4.80 g, 95%). LCMS [M+H]⁺ 193.2

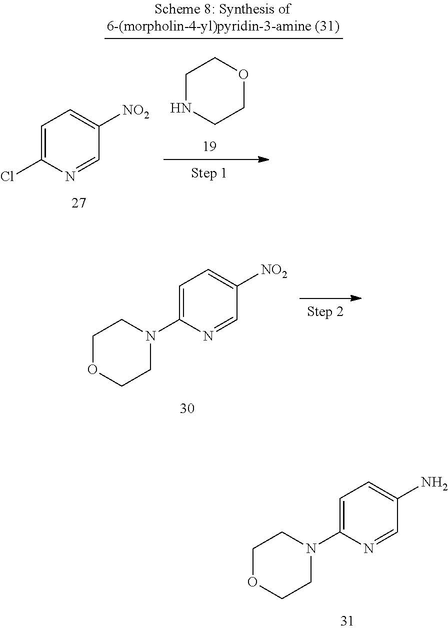 [SMILES: O=[N+]([O-])c1ccc(Cl)nc1 ; C1COCCN1 ; O=[N+]([O-])c1ccc(N2CCOCC2)nc1 ; Nc1ccc(N2CCOCC2)nc1]

### Step 1: Synthesis of 4-(5-nitropyridin-2-yl)morpholine (30)

To a stirred solution of morpholine (19) (1.63 mL, 18.9 mmol) and triethylamine (2.64 mL, 18.9 mmol) in dichloromethane (100 mL) at 0° C. was added 2-chloro-5-nitropyridine (27) (3.00 g, 18.9 mmol) and the reaction mixture was stirred at room temperature for 5 hours. Progress of the reaction was monitored TLC. Then the reaction was diluted with water (50 mL) and extracted with dichloromethane (30 mL×3). The combined organic layer was washed with brine (30 mL), dried over anhydrous sodium sulfate, filtered and evaporated to afford 4-(5-nitropyridin-2-yl)morpholine (30) (3.95 g, 18.9 mmol) as yellow solid. LCMS [M+H]⁺ 210.1

### Step 2: Synthesis of 6-(morpholin-4-yl)pyridin-3-amine (31)

To a stirred solution of 4-(5-nitropyridin-2-yl)morpholine (27) (4 g, 19.1 mmol) in ethanol (20.0 mL) was added palladium on carbon (0.4 g, 10% w/w) and the reaction mixture was subjected for hydrogenation using hydrogen bladder for 12 hours. The progress of the reaction was monitored by TLC and LCMS. After the reaction completion, the reaction mixture was filtered through celite bed and the filtrate was concentrated to afford 6-(morpholin-4-yl)pyridin-3-amine (31) (3.20 g, crude). [M+H]⁺ 180.1

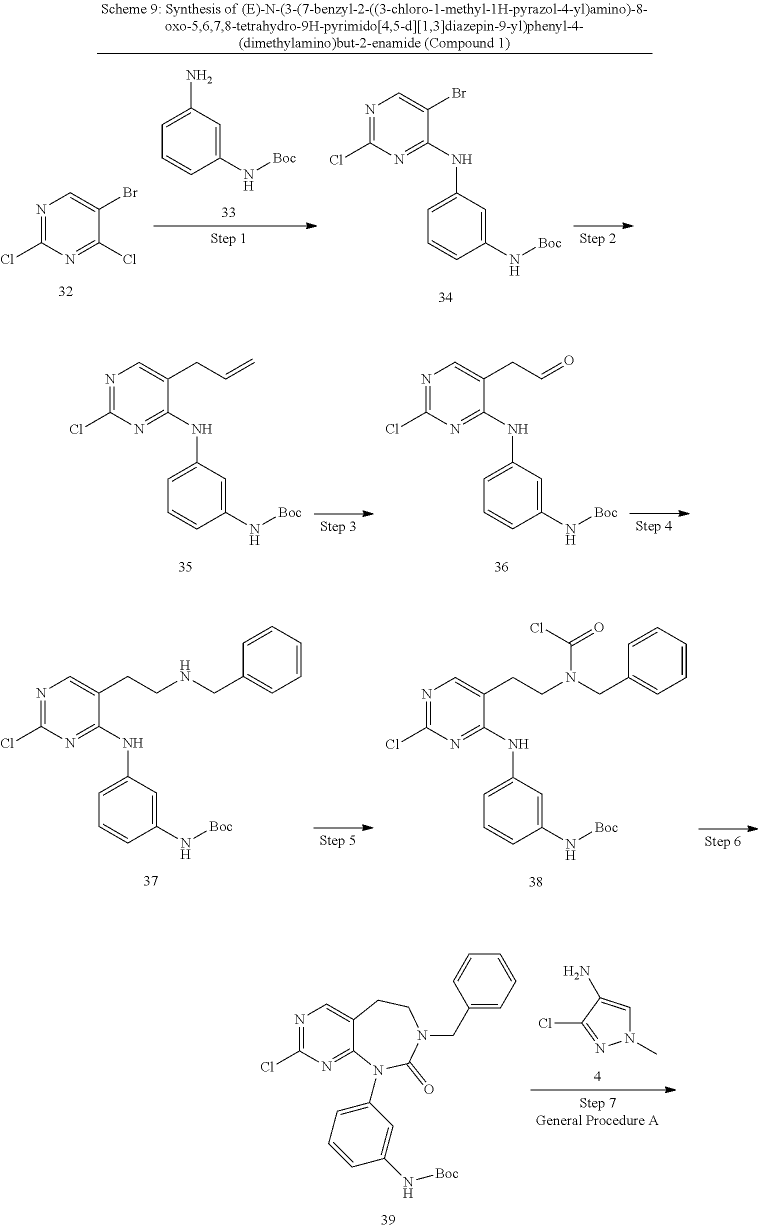

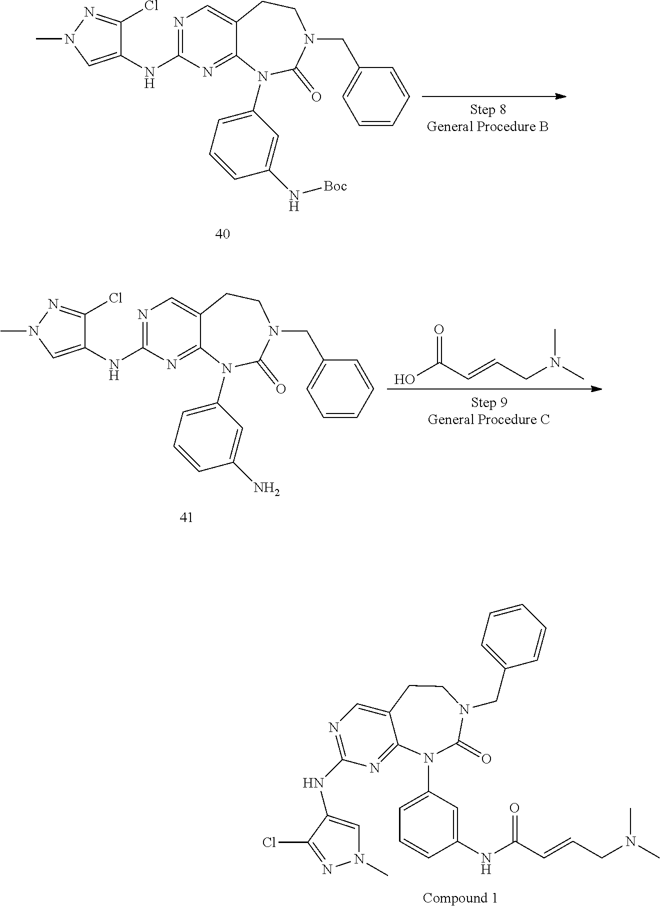

### Step 1: Synthesis of tert-butyl (3-((5-bromo-2-chloropyrimidin-4-yl)amino)phenyl)carbamate (34)

To a stirred solution of tert-butyl (3-aminophenyl)carbamate (33) (43.0 g, 206 mmol) in N,N-dimethylformamide (380 mL) were added 5-bromo-2,4-dichloropyrimidine (32) (47.0 g, 206 mmol) and potassium carbonate (57.0 g, 413 mmol) at room temperature. The resultant reaction mixture was stirred at room temperature for 16 hours. After completion of reaction (as per TLC monitoring), the reaction mixture was diluted with ice cold water (600 mL) and extracted with ethyl acetate (3×200 mL). The combined organic layer was washed with brine (200 mL), dried over anhydrous sodium sulphate, filtered and concentrated under reduced pressure. The crude product was purified by silica gel column chromatography using 50% ethyl acetate in heptane to afford the desired product (34) (75.0 g; Yield: 91%). ¹H NMR (400 MHz, DMSO-d₆): δ 9.42 (bs, 1H), 8.85 (s, 1H), 8.53 (s, 1H), 7.61 (s, 1H), 7.22-7.26 (m, 2H), 7.09 (s, 1H), 1.47 (s, 9H). LCMS [M+H]⁺ 399.20

### Step 2: Synthesis of tert-butyl (3-((5-allyl-2-chloropyrimidin-4-yl)amino)phenyl)carbamate (35)

To a stirred solution of tert-butyl (3-((5-bromo-2-chloropyrimidin-4-yl)amino)phenyl)carbamate (34) (20.0 g, 50.0 mmol) in N,N-dimethylformamide (90.0 mL) was added allyltributylstannane (19.9 g, 60.0 mmol) at room temperature. The resulting reaction mixture was purged with nitrogen for 30 minutes, then tetrakis(triphenylphosphine)palladium(0) (2.89 g, 2.50 mmol) and lithium chloride (2.76 g, 65.1 mmol) were added and the reaction mixture was heated at 100° C. for 2 hours. After completion of reaction (as per TLC monitoring), the reaction mixture was diluted by ice-cold water (400 mL) and extracted with ethyl acetate (3×200 mL). The combined organic layer was washed with brine (100 mL), dried over anhydrous sodium sulphate, filtered and concentrated under reduced pressure. The crude was purified by silica gel column chromatography using 10-30% ethyl acetate in heptane to get the desired product (35) (12.0 g; Yield: 66%). ¹H NMR (400 MHz, DMSO-d₆): δ 9.39 (bs, 1H), 8.82 (s, 1H), 7.99 (s, 1H), 7.72 (s, 1H), 7.19-7.26 (m, 2H), 7.12-7.14 (m, 1H), 5.92-6.00 (m, 1H), 5.12-5.16 (m, 2H), 3.38-3.40 (m, 2H), 1.47 (s, 9H). LCMS [M+H]⁺ 361.41

### Step 3: Synthesis of tert-butyl (3-((2-chloro-5-(2-oxoethyl)pyrimidin-4-yl)amino)phenyl)carbamate (36)

To a stirred solution of tert-butyl (3-((5-allyl-2-chloropyrimidin-4-yl)amino)phenyl)carbamate (35) (13.0 g, 36.0 mmol) in tetrahydrofuran (25.0 mL) and water (10.0 mL) was added sodium periodate (23.1 g, 108 mmol) portion wise at room temperature, followed by the addition of osmium tetroxide (1.00 mL, 0.36 mmol, 4% w/w in water). The resulting reaction mixture was stirred at room temperature for 4 hours. After completion of starting material (as a monitor by TLC) the solvent was evaporated under reduced pressure and the residue was diluted with ice cold water (100 mL) then extracted with ethyl acetate (3×100 mL). The combined organic layer was dried over sodium sulphate and concentrated under reduced pressure to get crude tert-butyl (3-((2-chloro-5-(2-oxoethyl)pyrimidin-4-yl)amino)phenyl)carbamate (36) (15.0 g, 37%) as a black gum, which was used as it is for the next step. LCMS [M+H]⁺ 363.37.

### Step 4: Synthesis tert-butyl (3-((5-(2-(benzylamino)ethyl)-2-chloropyrimidin-4-yl)amino)phenyl)carbamate (37)

To a stirred solution tert-butyl (3-((2-chloro-5-(2-oxoethyl)pyrimidin-4-yl)amino)phenyl)carbamate (36) (15.0 g) in methanol (150 mL) was added benzyl amine (11.8 mL, 107 mmol) at room temperature. The resultant reaction mixture was stirred at room temperature for 1.5 hour then sodium borohydride was added (5.3 g, 143 mmol) portion-wise at 0 to −5° C. and the reaction mixture was stirred at room temperature for 16 hours. After completion of starting material (as a monitored by TLC), the reaction mixture was concentrated under reduced pressure and then it was quenched with water (100 mL) and extracted with ethyl acetate (3×100 mL). The combined organic layer was dried over anhydrous sodium sulphate, filtered and concentrated under reduced pressure. The crude product was purified by reverse phase column chromatography (using C18 cartridge, 5 mm ammonium acetate in water/acetonitrile) to get the desired product (37) (2.10 g; Yield: 11%). LCMS [M+H]⁺ 454.37

### Step 5: Synthesis of tert-butyl (3-((5-(2-(benzyl(chlorocarbonyl)amino)ethyl)-2-chloropyrimidin-4-yl)amino)phenyl)carbamate (38)

To an ice-cold solution of tert-butyl (3-((5-(2-(benzylamino)ethyl)-2-chloropyrimidin-4-yl)amino)phenyl)carbamate (37) (2.10 g, 4.63 mmol) in tetrahydrofuran (20.0 mL) was added N,N-diisopropylethylamine (3.23 mL, 18.5 mmol) and triphosgene (0.55 g, 1.85 mmol). The reaction mixture was stirred for 15 minutes at same temperature. After completion of starting material (as monitored by TLC), saturated aqueous solution of sodium bicarbonate (30 mL) was added to the reaction mixture and extracted with dichloromethane (3×50 mL). The combined organic layer was dried over sodium sulfate, concentrated under reduced pressure to get tert-butyl (3-((5-(2-(benzyl(chlorocarbonyl)amino)ethyl)-2-chloropyrimidin-4-yl)amino)phenyl)carbamate (38) (2.10 g, Yield: 72%) as yellow solid. LCMS [M+H]⁺ 516.54.

### Step 6: Synthesis of tert-butyl (3-(7-benzyl-2-chloro-8-oxo-5,6,7,8-tetrahydro-9H-pyrimido[4,5-d][1,3]diazepin-9-yl)phenyl)carbamate (39)

To a stirred solution of tert-butyl (3-((5-(2-(benzyl(chlorocarbonyl)amino)ethyl)-2-chloropyrimidin-4-yl)amino)phenyl)carbamate (38) (1.80 g, 3.49 mmol) in acetonitrile (20.0 mL) were added N,N-dimethylpyridin-4-amine (0.255 g, 2.09 mmol) and triethylamine (1.41 g, 13.9 mmol) at room temperature. The reaction mixture was heated at 100° C. for 2 hours. After completion of starting materials (as monitor by TLC), ice cold water (25 mL) was added and extracted with dichloromethane (3×50 mL). The combined organic layer was dried over sodium sulphate, filtered and concentrated under reduced pressure. The crude product was washed with diethyl ether to get tert-butyl (3-(7-benzyl-2-chloro-8-oxo-5,6,7,8-tetrahydro-9H-pyrimido[4,5-d][1,3]diazepin-9-yl)phenyl)carbamate (39) (1.50 g, Yield: 66.35%) as a yellow solid. LCMS [M+H]⁺ 480.39.

### Step 7: Synthesis of tert-butyl (3-(7-benzyl-2-((3-chloro-1-methyl-1H-pyrazol-4-yl)amino)-8-oxo-5,6,7,8-tetrahydro-9H-pyrimido[4,5-d][1,3]diazepin-9-yl)phenyl)carbamate (40)

Title compound was prepared in a manner substantially similar to procedure mentioned in General Procedure A, to get tert-butyl (3-(7-benzyl-2-((3-chloro-1-methyl-1H-pyrazol-4-yl)amino)-8-oxo-5,6,7,8-tetrahydro-9H-pyrimido[4,5-d][1,3]diazepin-9-yl)phenyl)carbamate (40) as a green solid in 27% yield, which was used directly for the next step. LCMS [M+H]⁺ 575.26.

### Step 8: Synthesis of 9-(3-aminophenyl)-7-benzyl-2-((3-chloro-1-methyl-1H-pyrazol-4-yl)amino)-5,6,7,9-tetrahydro-8H-pyrimido[4,5-d][1,3]diazepin-8-one (41)

Title compound was prepared in a manner substantially similar to procedure mentioned in General Procedure B, to get 9-(3-aminophenyl)-7-benzyl-2-((3-chloro-1-methyl-1H-pyrazol-4-yl)amino)-5,6,7,9-tetrahydro-8H-pyrimido[4,5-d][1,3]diazepin-8-one (41) as a yellow solid (530 mg; Yield: 28%). LCMS [M+H]⁺ 475.40.

### Step 9: Synthesis of (E)-N-(3-(7-benzyl-2-((3-chloro-1-methyl-1H-pyrazol-4-yl)amino)-8-oxo-5,6,7,8-tetrahydro-9H-pyrimido[4,5-d][1,3]diazepin-9-yl)phenyl)-4-(dimethylamino)but-2-enamide (Compound 1)

Title compound was prepared in a manner substantially similar to procedure mentioned in General Procedure C, to get (E)-N-(3-(7-benzyl-2-((3-chloro-1-methyl-5H-pyrazol-4-yl)amino)-8-oxo-5,6,7,8-tetrahydro-9H-pyrimido[4,5-d][1,3]diazepin-9-yl)phenyl)-4-(dimethylamino)but-2-enamide Compound 1 as a white solid after prep-HPLC purification (13 mg, Yield: 7%). ¹H NMR (400 MHz, DMSO-d₆): 10.19 (s, 1H), 8.41 (bs, 1H), 8.16 (s, 1H), 7.80 (d, J 8.0 Hz, 1H), 7.67 (s, 1H), 7.42 (t, J 8.0 Hz, 1H), 7.28-7.37 (m, 5H), 7.08 (d, J=7.6 Hz, 1H), 6.70-6.76 (m, 1H), 6.39 (bs, 1H), 6.26 (d, J 15.2 Hz, 1H), 4.58 (s, 2H), 3.68 (s, 2H), 3.50 (s, 3H), 3.04 (d, J 5.2 Hz, 2H), 2.88 (s, 2H), 2.16 (s, 6H). LCMS [M+H]⁺ 586.38

The following compounds were prepared using the procedures described above:

| Cmpd | | Synthesis | LCMS | ¹H-NMR (400 |
|---|---|---|---|---|
| No. | Structure | Method | [M + H]⁺ | MHz, DMSO-d₆) |
| 2 | 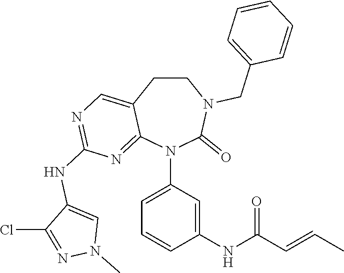 [SMILES: C/C=C/C(=O)Nc1cccc(N2C(=O)N(Cc3ccccc3)CCc3cnc(Nc4cn(C)nc4Cl)nc32)c1] | C | 543.28 | δ 10.05 (bs, 1H), 8.36 (bs, 1H), 8.14 (s, 1H), 7.79 (d, J = 8.0 Hz, 1H), 7.63 (s, 1H), 7.41-7.27 (m, 6H), 7.05 (d, J = 7.2 Hz, 1H), 6.80-6.75 (m, 1H), 6.39 (bs, 1H), 6.10 (d, J = 15.2 Hz, 1H), 4.57 (s, 2H), 3.66 (s, 2H), 3.48 (s, 3H), 2.86 (bs, 2H), 1.85 (d, J = 6.4 Hz, 3H). |
| 3 | 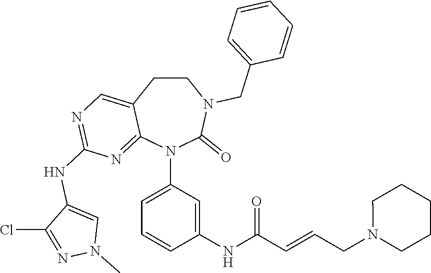 [SMILES: Cn1cc(Nc2ncc3c(n2)N(c2cccc(NC(=O)/C=C/CN4CCCCC4)c2)C(=O)N(Cc2ccccc2)CC3)c(Cl)n1] | C | 626.31 | δ 10.20 (bs, 1H), 8.44 (bs, 1H), 8.15 (s, 1H), 7.80 (d, J = 8.0 Hz, 1H), 7.67 (s, 1H), 7.65 (s, 1H), 7.42 (t, J = 8.4 Hz, 1H), 7.28-7.35 (m, 4H), 7.08 (d, J = 7.6 Hz, 1H), 6.70-6.76 (m, 1H), 6.37 (bs, 1H), 6.24 (d, J = 15.2 Hz, 1H), 4.58 (s, 2H), 3.67 (bs, 2H), 3.50 (s, 3H), 3.07 (d, J = 4.8 Hz, 2H), 2.88 (s, 2H), 2.33 (bs, |
| | | | | 4H), 1.50 (bs, 4H), |
| | | | | 1.38 (bs, 2H), |
| 4 | 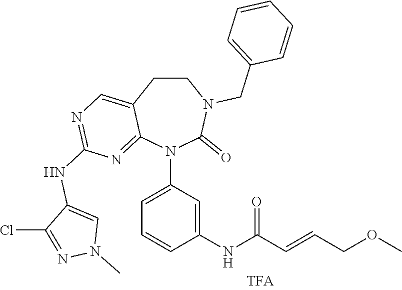 [SMILES: COC/C=C/C(=O)Nc1cccc(N2C(=O)N(Cc3ccccc3)CCc3cnc(Nc4cn(C)nc4Cl)nc32)c1] | C | 573.21 | δ 10.24 (bs, 1H), 8.61 (bs, 1H), 8.17 (s, 1H), 7.78 (d, J = 8.0 Hz, 1H), 7.69 (s, 1H), 7.42 (t, J = 8.0 Hz, 1H), 7.27- 7.37 (m, 6H), 7.08 (d, J = 7.6 Hz, 1H), 6.79 (dd, J = 15.6, 4.0 Hz, 1H), 6.40 (s, 1H), 6.32 (d, J = 15.2 Hz, 1H), 4.59 (s, 2H), 4.09-4.11 (m, 2H), 3.69 (bs, 2H), 3.51 (s, 3H), 3.32 (s, 3H), 2.90- 2.89 (m, 2H). |
| 5 | 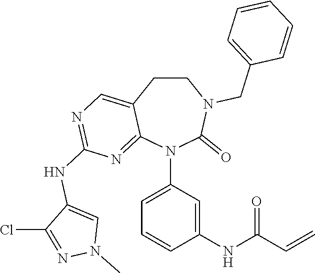 [SMILES: C=CC(=O)Nc1cccc(N2C(=O)N(Cc3ccccc3)CCc3cnc(Nc4cn(C)nc4Cl)nc32)c1] | D | 529.34 | δ 10.28 (bs, 1H), 8.42 (bs, 1H), 8.16 (s, 1H), 7.83 (d, J = 8.0 Hz, 1H), 7.65 (s, 1H), 7.41 (t, J = 8.0 Hz, 1H), 7.28- 7.37 (m, 5H), 7.10 (d, J = 8.0 Hz, 1H), 6.43 (dd, J = 10.0, 6.8 Hz, 2H), 6.25 (d, J = 16.8 Hz, 1H), 5.75 (d, J = 11.2 Hz, 1H), 4.58 (s, 2H), 3.67-3.67 (m, 2H), 3.50 (s, 3H), 2.90-2.87 (m, 2H). |
| 6 | 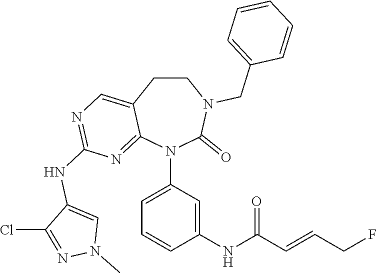 [SMILES: Cn1cc(Nc2ncc3c(n2)N(c2cccc(NC(=O)/C=C/CF)c2)C(=O)N(Cc2ccccc2)CC3)c(Cl)n1] | C | 561.14 | δ 10.32 (bs, 1H), 8.45 (bs, 1H), 8.16 (s, 1H), 7.80 (d, J = 8.8 Hz, 1H), 7.67 (s, 1H), 7.41 (t, J = 8.0 Hz, 1H), 7.28- 7.37 (m, 5H), 7.08 (d, J = 7.6 Hz, 1H), 6.82-6.92 (m, 1H), 6.34 (d, J = 14.8 Hz, 2H), 5.18 (d, J = 46.4 Hz, 2H), 4.58 (s, 2H), 3.67- 3.68 (m, 2H), 3.50 (s, 3H), 2.88 (s, 2H). |
| 7 | 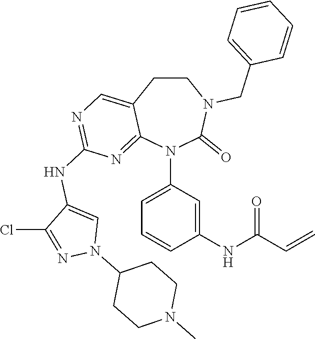 [SMILES: C=CC(=O)Nc1cccc(N2C(=O)N(Cc3ccccc3)CCc3cnc(Nc4cn(C5CCN(C)CC5)nc4Cl)nc32)c1] | D | 612.29 | δ 10.31 (bs, 1H), 8.40 (bs, 1H), 8.17 (s, 1H), 7.83 (d, J = 7.2 Hz, 1H), 7.66 (s, 1H), 7.39 (t, J = 8.0 Hz, 1H), 7.26- 7.33 (m, 5H), 7.07 (d, J = 8.4 Hz, 1H), 6.40-6.53 (m, 2H), 6.24-6.28 (m, 1H), 5.75 (d, J = 10.0 Hz, 1H), 4.58 (s, 2H), 3.66-3.69 (m, 2H), 3.55-3.59 (m, 1H), 2.87- 2.90 (m, 2H), 2.78- 2.80 (m, 2H), 2.20 (s, 3H), 2.01-2.06 (m, 2H), 1.78-1.85 (m, 2H), 1.57- 1.60 (m, 2H). |
| 8 | 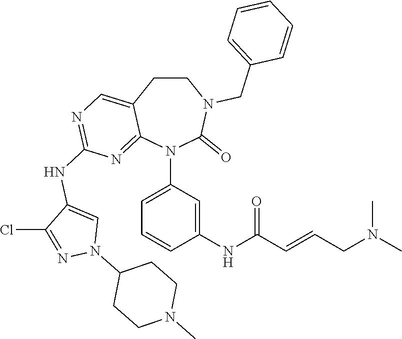 [SMILES: CN(C)C/C=C/C(=O)Nc1cccc(N2C(=O)N(Cc3ccccc3)CCc3cnc(Nc4cn(C5CCN(C)CC5)nc4Cl)nc32)c1] | C | 669.29 | δ 10.23 (bs, 1H), 8.39 (bs, 1H), 8.17 (s, 1H), 7.79 (d, J = 8.0 Hz, 1H), 7.67 (s, 1H), 7.37 (t, J = 8.0 Hz, 1H), 7.26- 7.33 (m, 5H), 7.04 (d, J = 8.0 Hz, 1H), 6.70-6.77 (m, 1H), 6.52-6.56 (m, 1H), 6.24 (d, J = 15.6 Hz, 1H), 4.57 (s, 2H), 3.66-3.69 (m, 2H), 3.54-3.56 (m, 1H), 3.04- 3.06 (m, 2H), 2.87- 2.89 (m, 2H), 2.77- 2.80 (m, 2H), 2.20 (s, 3H), 2.16 (s, 6H), 2.00-2.08 (m, 2H), 1.76- |
| | | | | 1.85 (m, 2H), 1.57- |
| | | | | 1.60 (m, 2H). |
| 9 | 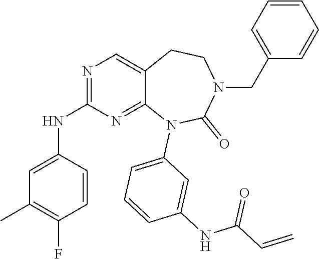 [SMILES: C=CC(=O)Nc1cccc(N2C(=O)N(Cc3ccccc3)CCc3cnc(Nc4ccc(F)c(C)c4)nc32)c1] | D | 523.4 | δ 10.23 (s, 1H), 9.20 (s, 1H), 8.18 (s, 1H), 7.81 (d, J = 8.0 Hz, 1H), 7.70 (s, 1H), 7.42-7.29 (m, 6H), 7.08 (d, J = 7.6 Hz, 1H), 6.93 (d, J = 5.6 Hz, 1H), 6.82 (s, 1H), 6.55- 6.50 (m, 1H), 6.46- 6.39 (m, 1H), 6.27- 6.23 (m, 1H), 5.75 (d, J = 10.0 Hz, 1H), 4.60 (s, 2H), 3.69 (s, 2H), 2.89 (s, 2H), 2.07 (s, 3H). |
| 10 | 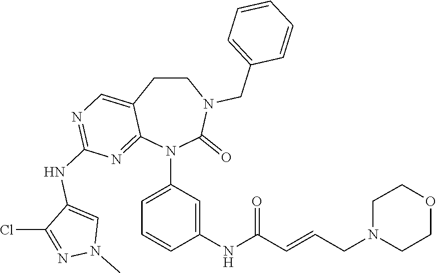 [SMILES: Cn1cc(Nc2ncc3c(n2)N(c2cccc(NC(=O)/C=C/CN4CCOCC4)c2)C(=O)N(Cc2ccccc2)CC3)c(Cl)n1] | C | 628.3 | δ 10.18 (s, 1H), 8.42 (s, 1H), 8.15 (s, 1H), 7.79 (d, J = 7.6 Hz, 1H), 7.64 (s, 1H), 7.27-7.43 (m, 6H), 7.07 (d, J = 8.0 Hz, 1H), 6.71 (d, J = 15.2 Hz, 1H), 6.26 (d, J = 15.2 Hz, 2H), 4.57 (s, 2H), 3.67 (s, 2H), 3.58 (s, 3H), 3.48 (s, 2H), 3.30 (s, 2H merged with DMSO peak), 3.11 (d, J = 5.2 Hz, 2H), 2.87 (s, 2H), 2.37 (s, 4H). |
| 11 | 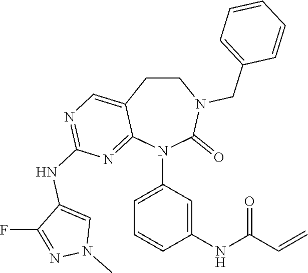 [SMILES: C=CC(=O)Nc1cccc(N2C(=O)N(Cc3ccccc3)CCc3cnc(Nc4cn(C)nc4F)nc32)c1] | D | 513.3 | δ 10.26 (s, 1H), 8.92 (s, 1H), 8.11 (s, 1H), 7.82 (d, J = 8.0 Hz, 1H), 7.64 (s, 1H), 7.43-7.26 (m, 6H), 7.08 (d, J = 8.0 Hz, 1H), 6.44- 6.38 (m, 1H), 6.25- 6.21 (m, 2H), 5.75- 5.73 (m, 1H), 4.56 (s, 2H), 3.66 (s, 2H), 3.39 (s, 3H), 2.85 (s, 2H). |
| 12 | 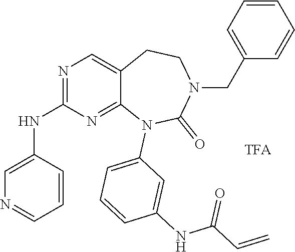 [SMILES: C=CC(=O)Nc1cccc(N2C(=O)N(Cc3ccccc3)CCc3cnc(Nc4cccnc4)nc32)c1] | D | 492.4 | δ 10.33 (s, 1H), 8.97 (s, 1H), 8.77 (s, 1H), 8.38 (s, 1H), 7.86 (s, 1H), 7.72 (s, 2H), 7.55-7.53 (m, 1H), 7.46-7.45 (m, 1H), 7.36-7.31 (m, 4H), 7.19- 7.17 (m, 2H), 6.78 (s, 2H), 6.45-6.44 (m, 1H), 6.28-6.27 (m, 1H), 5.78 (d, J = 10.0 Hz, 1H), 4.64 (s, 2H), 3.82 (s, 2H), 3.14 (s, 2H). |
| 13 | 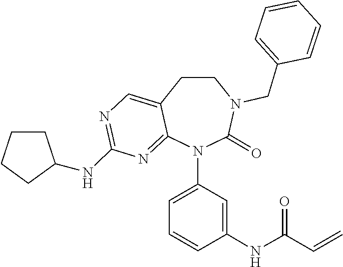 [SMILES: C=CC(=O)Nc1cccc(N2C(=O)N(Cc3ccccc3)CCc3cnc(NC4CCCC4)nc32)c1] | E | 483.0 | δ 10.18 (s, 1H), 7.99 (s, 1H), 7.72 (s, 1H), 7.58 (bs, 2H), 7.28-7.35 (m, 5H), 6.99-7.02 (m, 1H), 6.32-6.46 (m, 2H), 6.21-6.25 (m, 1H), 5.72- 5.75 (m, 1H), 4.58 (s, 2H), 3.68 (bs, 2H), 2.81 (bs, 2H), 2.49 (1H, merged with DMSO peak), 1.47 (bs, 4H), 1.15- 1.22 (m, 4H) |
| 14 | 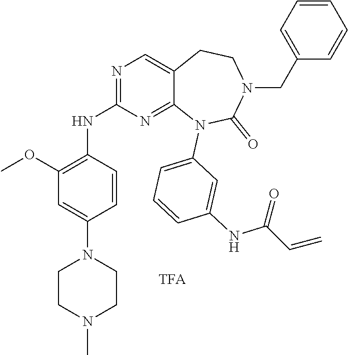 [SMILES: C=CC(=O)Nc1cccc(N2C(=O)N(Cc3ccccc3)CCc3cnc(Nc4ccc(N5CCN(C)CC5)cc4OC)nc32)c1] | D | 619.48 | δ 10.31 (s, 1H), 9.64 (bs, 1H), 8.14 (s, 1H), 8.04 (d, J = 7.2 Hz, 1H), 7.60 (bs, 1H), 7.50 (s, 1H), 7.40-7.27 (m, 6H), 7.03 (d, J = 8.0 Hz, 1H), 6.82 (d, J = 8.0 Hz, 1H), 6.54 (s, 1H), 6.41 (q, J = 12.0 Hz, 1H), 6.26 (d, J = 16.4 Hz, 1H), 5.98 (d, J = 8.0 Hz, 1H), 5.74 (d, J = 10.0 Hz, 1H), 4.58 (s, 2H), 3.76 (s, 3H), 3.68-3.63 (m, 6H), 3.10 (bs, 2H). 2.78-2.85 (m, 7H). |
| 15 | 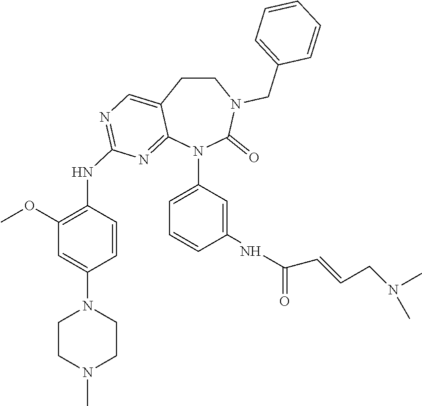 | C₁ | 674.5 [M − H]⁻ | δ 10.19 (s, 1H), 8.11 (s, 1H), 7.98 (d, J = 8.0 Hz, 1H), 7.59 (s, 1H), 7.38- 7.27 (m, 7H), 6.99 (d, J = 7.6 Hz, 1H), 6.81 (d, J = 8.8 Hz, 1H), 6.74-6.70 (m, 1H), 6.46 (d, J = 2.4 Hz, 1H), 6.24 (d, J = 15.2 Hz, 1H), 5.95 (d, J = 8.8 Hz, 1H), 4.58 (s, 2H), 3.76 (s, 3H), 3.68 (s, 2H), 3.03 (d, J = 5.6 Hz, 2H), 2.96 (t, J = 8.8 Hz, 4H), 2.87 (d, J = 4.4 Hz, 2H), 2.40 (d, J = 4.4 Hz, 4H), 2.20 (s, 9H) |
| 16 | 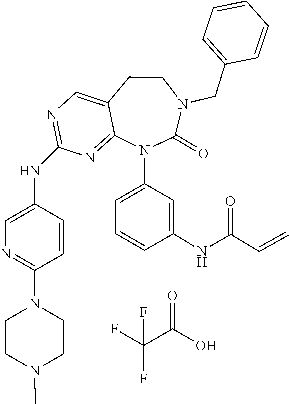 [SMILES: C=CC(=O)Nc1cccc(N2C(=O)N(Cc3ccccc3)CCc3cnc(Nc4ccc(N5CCN(C)CC5)nc4)nc32)c1 ; O=C(O)C(F)(F)F] | D | 588.4 [M − H]⁻ | δ 10.29 (s, 1H), 9.58 (bs, 1H), 9.22 (s, 1H), 8.15 (s, 1H), 8.01 (d, J = 10.0 Hz, 1H), 7.91 (s, 1H), 7.51 (s, 1H), 7.41- 7.26 (m, 4H), 7.17 (d, J = 12.2 Hz, 1H), 7.07 (d, J = 8.0 Hz, 1H), 6.94 (s, 1H), 6.46-6.37 (m, 2H), 6.26 (d, J = 14.8 Hz, 1H), 5.75 (d, J = 11.6 Hz, 1H), 4.59 (s, 2H), 4.15 (bs, 6H), 3.69 (t, J = 9.2 Hz, 2H), 3.47 (d, J = 8.0 Hz, 2H), 3.02-2.83 (m, 5H). |
| 17 | 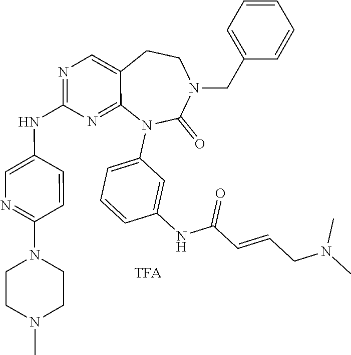 [SMILES: CN(C)C/C=C/C(=O)Nc1cccc(N2C(=O)N(Cc3ccccc3)CCc3cnc(Nc4ccc(N5CCN(C)CC5)nc4)nc32)c1] | C₁ | 645.5 [M − H]⁻ | δ 10.46 (s, 1H), 9.96 (s, 1H), 9.23 (s, 1H), 8.16 (s, 1H), 7.92-7.89 (m, 2H), 7.57 (s, 1H), 7.41 (t, J = 8.0 Hz, 1H), 7.33-7.28 (m, 5H), 7.22 (bs, 1H), 7.08 (d, J = 8.0 Hz, 1H), 6.74-6.70 (m, 1H), 6.44 (d, J = 16.0 Hz, 2H), 4.58 (s, 2H), 4.17 (bs, 2H), 3.94 (s, 2H), 3.69 (bs, 2H), 3.46 (bs, 2H), 3.04 (bs, 4H), 2.78 (bs, 11H). |
| 18 | 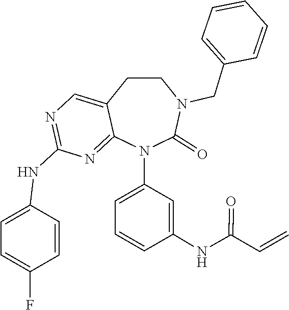 [SMILES: C=CC(=O)Nc1cccc(N2C(=O)N(Cc3ccccc3)CCc3cnc(Nc4ccc(F)cc4)nc32)c1] | D | 509.3 | δ 10.25 (s, 1H), 9.33 (s, 1H), 8.19 (s, 1H), 7.85 (d, J = 8.0 Hz, 1H), 7.71 (s, 1H), 7.25-7.50 (m, 6H), 7.09 (d, J = 8.0 Hz, 1H), 7.01 (bs, 2H), 6.70 (t, J = 14.0 Hz, 2H), 6.35-6.45 (m, 1H), 6.25 (d, J = 18.0 Hz, 1H), 5.85 (d, J = 8.0 Hz, 1H), 4.61 (s, 2H), 3.81 (s, 2H), 2.90 (s, 2H). |
| 19 | 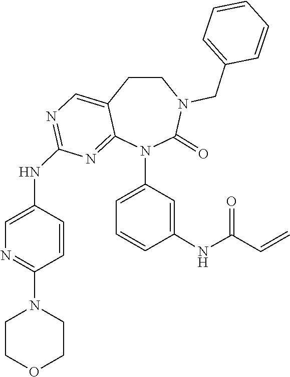 [SMILES: C=CC(=O)Nc1cccc(N2C(=O)N(Cc3ccccc3)CCc3cnc(Nc4ccc(N5CCOCC5)nc4)nc32)c1] | E | 577.4 | δ 10.26 (s, 1H), 9.12 (s, 1H), 8.13 (s, 1H), 7.99 (d, J = 8.0 Hz, 1H), 7.88 (s, 1H), 7.55 (s, 1H), 7.38-7.27 (m, 5H), 7.02-7.00 (m, 2H), 6.50-6.35 (m, 2H), 6.26-6.20 (m, 2H), 5.74 (d, J = 9.6 Hz, 1H), 4.58 (s, 2H), 3.65 (bs, 6H), 3.19 (bs, 4H), 2.86 (s, 2H) |
| 20 | 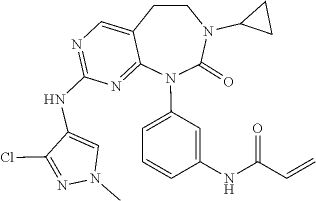 [SMILES: C=CC(=O)Nc1cccc(N2C(=O)N(C3CC3)CCc3cnc(Nc4cn(C)nc4Cl)nc32)c1] | E | 479.2 | δ 10.25 (s, 1H), 8.44-8.39 (m, 1H), 8.17 (s, 1H), 7.78 (d, J = 7.6 Hz, 1H), 7.60 (s, 1H), 7.39 (t, J = 7.8 Hz, 1H), 7.02 (d, J = 8.0 Hz, 1H), 6.45-6.38 (m, 2H), 6.23 (d, J = 16.4 Hz, 1H), 5.74 (d, J = 10.4 Hz, 1H), 3.69 (s, 2H), 3.49 (s, 3H), 2.91 (s, |
| | | | | 2H), 2.74 (s, 1H), |
| | | | | 0.69 (d, J = 5.6 Hz, |
| | | | | 2H), 0.60 (s, 2H). |
| 21 | 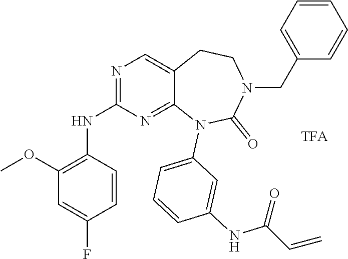 [SMILES: C=CC(=O)Nc1cccc(N2C(=O)N(Cc3ccccc3)CCc3cnc(Nc4ccc(F)cc4OC)nc32)c1] | E | 539.3 | δ 10.24 (s, 1H), 8.17 (s, 1H), 7.80 (d, J = 8.0 Hz, 1H), 7.67 (s, 1H), 7.53 (s, 1H), 7.41-7.27 (m, 6H), 7.05 (d, J = 7.2 Hz, 1H), 6.94 (t, J = 7.4 Hz, 1H), 6.80 (d, J = 10.4 Hz, 1H), 6.44-6.37 (m, 1H), 6.25-6.14 (m, 2H), 5.73 (d, J = 10.0 Hz, 1H), 4.58 (s, 2H), 3.78 (s, 3H), 3.68 (s, 2H), 2.88 (s, 2H). |
| 22 | 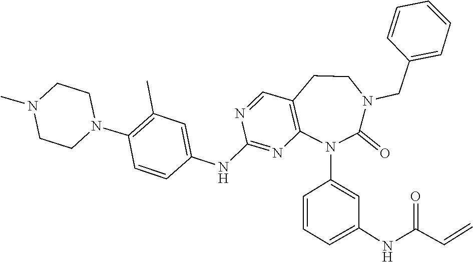 [SMILES: C=CC(=O)Nc1cccc(N2C(=O)N(Cc3ccccc3)CCc3cnc(Nc4ccc(N5CCN(C)CC5)c(C)c4)nc32)c1] | E | 603.5 | δ 10.24 (s, 1H), 9.02 (s, 1H), 8.12 (s, 1H), 7.97 (d, J = 7.6 Hz, 1H), 7.60 (s, 1H), 7.26-7.38 (m, 6H), 7.01 (d, J = 7.2 Hz, 1H), 6.80 (s, 1H), 6.72 (d, J = 8.8 Hz, 1H), 6.37- 6.47 (m, 2H), 6.23 (d, J = 16.8 Hz, 1H), 5.72 (d, J = 10.0 Hz, 1H), 4.56 (s, 2H), 3.67 (s, 2H), 2.85 (s, 2H), 2.63 (s, 4H), 2.30-2.48 |
| | | | | (m, 4H), 2.20 (s, |
| | | | | 3H), 2.02 (s, 3H) |
| 23 | 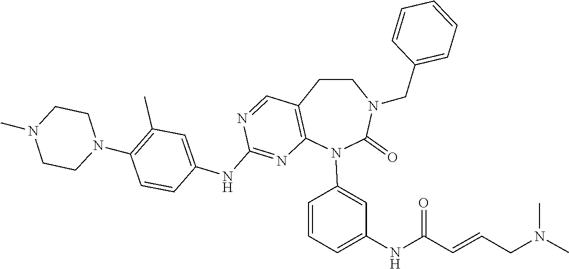 | C | 660.6 | δ 10.16 (s, 1H), 9.02 (s, 1H), 8.12 (s, 1H), 7.95 (d, J = 8.0 Hz, 1H), 7.62 (s, 1H), 7.37-7.27 (m, 6H), 7.99 (d, J = 7.6 Hz, 1H), 6.80 (s, 1H), 6.72 (t, J = 8.4 Hz, 2H), 6.46 (d, J = 8.8 Hz, 1H), 6.24 (d, J = 15.2 Hz, 1H), 4.57 (s, 2H), 3.67 (s, 2H), 3.02 (d, J = 5.2 Hz, 2H), 2.86 (s, 2H), 2.64 (s, 3H), 2.39 (s, 3H), 2.21-2.15 (m, 8H), |
| | | | | 2.03 (s, 3H), 1.86 |
| | | | | (s, 3H) |
| 24 | 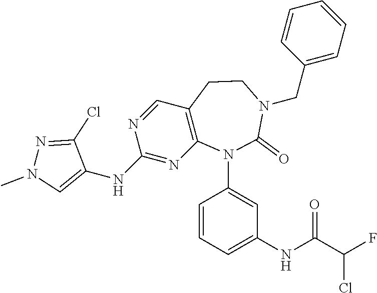 [SMILES: Cn1cc(Nc2ncc3c(n2)N(c2cccc(NC(=O)C(F)Cl)c2)C(=O)N(Cc2ccccc2)CC3)c(Cl)n1] | C | 571.27 | δ 10.75 (s, 1H), 8.37 (s, 1H), 8.15 (s, 1H), 7.76 (d, J = 8.0 Hz, 1H), 7.66 (s, 1H), 7.46 (d, J = 7.6 Hz, 1H), 7.32- 7.27 (m, 5H), 7.15 (d, J = 7.6 Hz, 1H), 6.84 (d, J = 48.0 Hz, 1H), 6.38 (bs, 1H), 4.57 (s, 2H), 3.68 (s, 2H), 3.50 (s, 3H), 2.87 (s, 2H) |
| 25 | 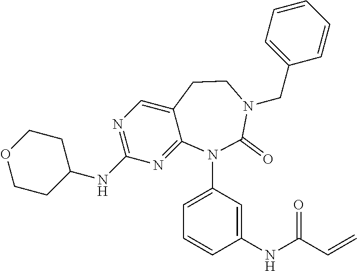 [SMILES: C=CC(=O)Nc1cccc(N2C(=O)N(Cc3ccccc3)CCc3cnc(NC4CCOCC4)nc32)c1] | E | 499.3 | δ 10.14 (s, 1H), 7.95 (s, 1H), 7.68 (s, 1H), 7.58 (d, J = 8.0 Hz, 1H), 7.26- 7.31 (m, 6H), 6.96 (d, J = 7.2 Hz, 1H), 6.63 (bs, 1H), 6.38-6.45 (m, 1H), 6.22 (d, J = 8.2 Hz, 1H), 5.72 (d, J = 10.0 Hz, 1H), 4.55 (s, 2H), 3.62 (s, 4H), 2.91 (bs, 3H), 2.77 (s, 2H), 1.37 (s, 2H), 1.15 (s, 2H) |
| 26 | 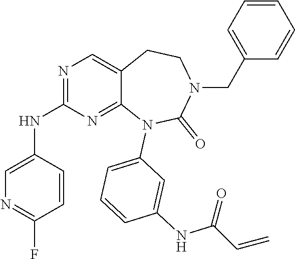 [SMILES: C=CC(=O)Nc1cccc(N2C(=O)N(Cc3ccccc3)CCc3cnc(Nc4ccc(F)nc4)nc32)c1] | D | 510.3 | δ 10.24 (s, 1H), 9.57 (s, 1H), 8.21 (s, 1H), 7.86 (s, 1H), 7.76 (d, J = 8.0 Hz, 1H), 7.69 (s, 1H), 7.27-7.43 (m, 7H), 7.07 (d, J = 7.6 Hz, 1H), 6.52 (d, J = 8.8 Hz, 1H), 6.37- 6.43 (m, 1H), 6.22 (d, J = 8.2 Hz, 1H), 5.72 (d, J = 10.4 Hz, 1H), 4.58 (s, 2H). 3.69 (s, 2H), 2.89 (s, 2H). |
| 27 | 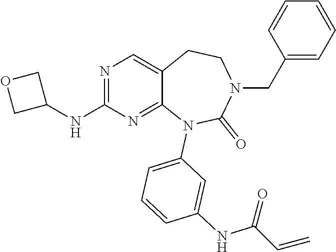 [SMILES: C=CC(=O)Nc1cccc(N2C(=O)N(Cc3ccccc3)CCc3cnc(NC4COC4)nc32)c1] | D | 471.3 | δ 10.15 (s, 1H), 8.0 (s, 1H), 7.61 (d, J = 8.0 Hz, 2H), 7.34-7.26 (m, 6H), 6.98 (d, J = 7.2 Hz, 1H), 6.45 (bs, 2H), 6.23 (d, J = 17.2 Hz, 1H), 5.73 (d, J = 9.6 Hz, 1H), 4.54 (s, 2H), 4.15 (bs, 4H), 3.62 (s, 2H), 3.27 (1H merged with DMSO peak), 2.79 (s, 2H) |

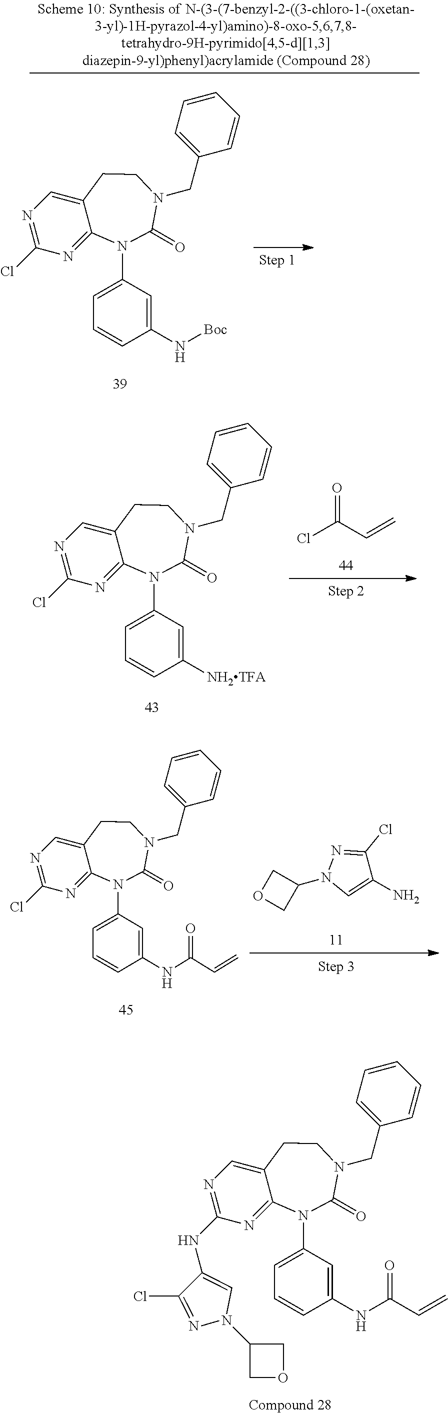

### Step 1: Synthesis of 7-benzyl-2-chloro-9-(3-((2,2,2-trifluoroacetyl)-14-azaneyl)phenyl)-5,6,7,9-tetrahydro-8H-pyrimido[4,5-d][1,3]diazepin-8-one (43)

To a stirred solution of tert-butyl N-(3-{7-benzyl-2-chloro-8-oxo-5H,6H,7H,8H,9H-pyrimido[4,5-d][1,3]diazepin-9-yl}phenyl)carbamate (39) (0.5 g, 1.04 mmol) in dichloromethane (10 mL) was added trifluoroacetic acid (4.0 mL) at 0° C. and the reaction was stirred at room temperature for 4 hours. Progress of the reaction was monitored by TLC. After completion of reaction, the reaction mixture was concentrated under reduced pressure. The crude product was triturated with diethyl ether (10 mL) and dried to afford N-(3-{7-benzyl-2-chloro-8-oxo-5H,6H,7H,8H,9H-pyrimido[4,5-d][1,3]diazepin-9-yl}phenyl)-2,2,2-trifluoroacetamide (43) (0.49 g, 98.95%) as pale yellow solid. LCMS [M+H]⁺ 380.1

### Step 2: Synthesis of N-(3-(7-benzyl-2-chloro-8-oxo-5,6,7,8-tetrahydro-9H-pyrimido[4,5-d][1,3]diazepin-9-yl)phenyl)acrylamide (45)

Title compound was prepared in a manner substantially similar to procedure mentioned in General Procedure E, to get the desired product (45) (0.36 g, 98.7%) as pale yellow solid. LCMS [M+H]⁺ 434.1

### Step 3: Synthesis of N-(3-(7-benzyl-2-((3-chloro-1-(oxetan-3-yl)-1H-pyrazol-4-yl)amino)-8-oxo-5,6,7,8-tetrahydro-9H-pyrimido[4,5-d][1,3]diazepin-9-yl)phenyl)acrylamide (Compound 28)

To a stirred solution of N-(3-{7-benzyl-2-chloro-8-oxo-5H,6H,7H,8H,9H-pyrimido[4,5-d][1,3]diazepin-9-yl}phenyl)prop-2-enamide (45) (0.15 g, 0.35 mmol) in tetrahydrofuran (3.0 mL) was added 3-chloro-1-(oxetan-3-yl)-1H-pyrazol-4-amine (11) (0.072 g, 0.415 mmol), cesium carbonate (0.34 g, 1.04 mmol), rac-BINAP (0.043 g, 0.069 mmol) and the reaction mixture was purged under nitrogen for 5 minutes. Then tris(dibenzylideneacetone)dipalladium(0) (0.032 g, 0.035 mmol) was added and the reaction was stirred at 100° C. for 16 hours in a sealed tube. Progress of the reaction was monitored by TLC and LCMS. After completion of the reaction, the reaction mixture was diluted with ethyl acetate (100 mL) and washed with water (10 mL). Organic layer was washed with brine (20 mL), dried over anhydrous sodium sulfate, filtered and concentrated under reduced pressure. The crude product was purified by preparative HPLC to afford N-[3-(7-benzyl-2-{[3-chloro-1-(oxetan-3-yl)-1H-pyrazol-4-yl]amino}-8-oxo-5H,6H,7H,8H,9H-pyrimido[4,5-d][1,3]diazepin-9-yl)phenyl]prop-2-enamide (Compound 28) (0.013 g, 6.6%) as off white solid. ¹H NMR (400 MHz, DMSO-d6) δ 10.28 (s, 1H), 8.51 (bs, 1H), 8.15 (s, 1H), 7.84 (d, J=8.0 Hz, 1H), 7.62 (s, 1H), 7.41 (t, J=7.8 Hz, 1H), 7.33-7.27 (m, 5H), 7.07 (d, J=8.0 Hz, 1H), 6.45-6.39 (m, 2H), 6.28-6.24 (m, 1H), 5.76 (d, J=9.6 Hz, 1H), 5.01 (bs, 1H), 4.81 (t, J=6.8 Hz, 2H), 4.70-4.69 (m, 2H), 4.56 (s, 2H), 3.66 (s, 2H), 2.87 (s, 2H); LCMS [M+H]⁺ 571.3

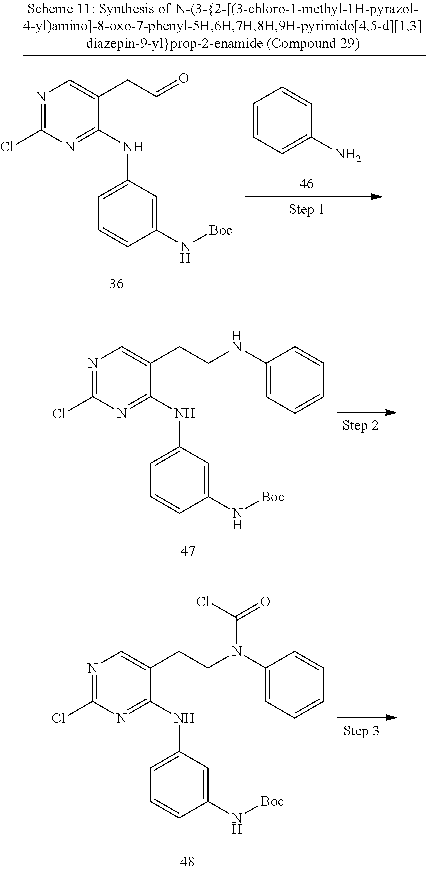 [SMILES: CC(C)(C)OC(=O)Nc1cccc(Nc2nc(Cl)ncc2CC=O)c1 ; Nc1ccccc1 ; CC(C)(C)OC(=O)Nc1cccc(Nc2nc(Cl)ncc2CCNc2ccccc2)c1 ; CC(C)(C)OC(=O)Nc1cccc(Nc2nc(Cl)ncc2CCN(C(=O)Cl)c2ccccc2)c1]

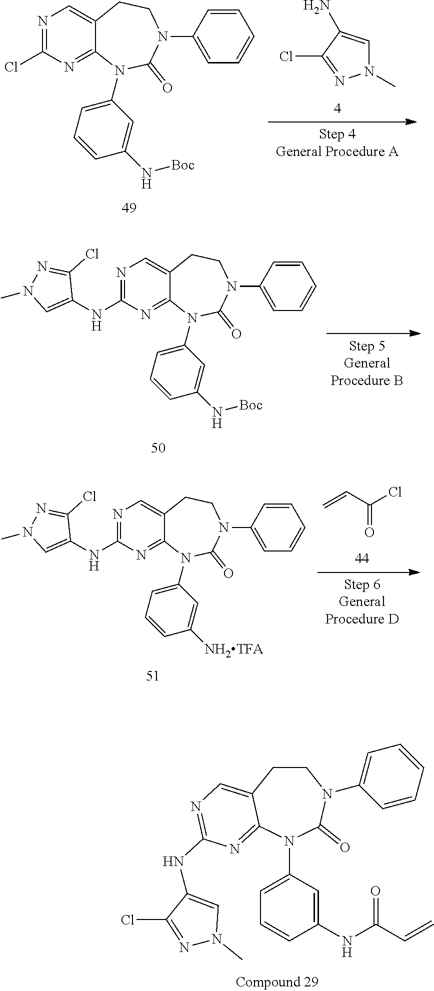

### Step 1: Synthesis of tert-butyl N-[3-({2-chloro-5-[2-(phenylamino)ethyl]pyrimidin-4-yl}amino)phenyl]carbamate (47)

To a stirred solution of tert-butyl N-(3-{[2-chloro-5-(2-oxoethyl)pyrimidin-4-yl]amino}phenyl)carbamate (36) (5.00 g, 13.8 mmol) in ethanol (75.0 mL) at room temperature were added aniline (46) (5.03 mL, 55.1 mmol) and molecular sieves (5 g). The reaction mixture was stirred at 70° C. for 5 hours and then cooled to 0° C., sodium borohydride (0.54 g, 15.2 mmol) was added. Then the reaction mixture was stirred at room temperature for 12 hours. After completion of reaction, the reaction mixture was filtered through celite and the filtrate was evaporated under reduced pressure. The crude product was purified by flash column chromatography using combiflash purifier and was eluted with 40-80% ethyl acetate in hexane to give the title compound (47) (4.0 g, 66%) as white solid. LCMS [M+H]⁺ 440.2

### Step 2: Synthesis of tert-butyl N-{3-[(2-chloro-5-{2-[(chlorocarbonyl)(phenyl)amino]ethyl}pyrimidin-4-yl)amino]phenyl}carbamate (48)

To a stirred solution of tert-butyl N-[3-({2-chloro-5-[2-(phenylamino)ethyl]pyrimidin-4-yl}amino)phenyl]carbamate (47) (4.30 g, 9.77 mmol) and N,N-diisopropylethylamine (6.81 mL, 39.1 mmol) in tetrahydrofuran (40.0 mL) at 0° C. was added triphosgene (1.16 g, 3.91 mmol). Then the reaction mixture was stirred at 0° C. for 15 minutes. After completion of starting material (as monitor by TLC), the reaction mixture was quenched with saturated aqueous sodium bicarbonate solution (30 mL) and was extracted with ethyl acetate (3×50 mL). The combined organic layer was washed with brine, dried over sodium sulfate, concentrated under reduced pressure to afford the desired compound (48) as a pasty solid and it was taken for next step without any purification.

### Step 3: Synthesis of tert-butyl N-(3-{2-chloro-8-oxo-7-phenyl-5H,6H,7H,8H,9H-pyrimido[4,5-d][1,3]diazepin-9-yl}phenyl)carbamate (49)

To a stirred solution of tert-butyl N-{3-[(2-chloro-5-{2-[(chlorocarbonyl)(phenyl)amino]ethyl}pyrimidin-4-yl)amino]phenyl}carbamate (48) (5.00 g, 9.95 mmol) in acetonitrile (40.0 mL) were added 4-dimethylaminopyridine (0.730 g, 5.97 mmol) and N,N-diisopropylethylamine (6.93 mL, 39.8 mmol) at room temperature. The reaction mixture was stirred at 100° C. for 2 hours. After completion of starting material (as monitor by TLC), ice cold water (50 mL) was added and extracted with ethyl acetate (3×50 mL). The combined organic layer was washed with brine, dried over sodium sulfate, concentrated under reduced pressure. The crude product was purified by column chromatography by using silica column with 60% ethyl acetate in hexane as an eluent to give the tert-butyl N-(3-{2-chloro-8-oxo-7-phenyl-5H,6H,7H,8H,9H-pyrimido[4,5-d][1,3]diazepin-9-yl}phenyl)carbamate (49) (3.5 g, 75%) as light yellow solid. LCMS [M+H]⁺ 466.2.

### Step 4: Synthesis of tert-butyl N-(3-{2-[(3-chloro-1-methyl-1H-pyrazol-4-yl)amino]-8-oxo-7-phenyl-5H,6H,7H,8H,9H-pyrimido[4,5-d][1,3]diazepin-9-yl}phenyl)carbamate (50)

Title compound was prepared in a manner substantially similar to procedure mentioned in General Procedure A, to get tert-butyl N-(3-{2-[(3-chloro-1-methyl-1H-pyrazol-4-yl)amino]-8-oxo-7-phenyl-5H,6H,7H,8H,9H-pyrimido[4,5-d][1,3]diazepin-9-yl}phenyl)carbamate (50) as off white solid, which was used directly for the next step. LCMS [M+H]⁺ 561.2.

### Step 5: Synthesis of N-(3-{2-[(3-chloro-1-methyl-1H-pyrazol-4-yl)amino]-8-oxo-7-phenyl-5H,6H,7H,8H,9H-pyrimido[4,5-d][1,3]diazepin-9-yl}phenyl)-2,2,2-trifluoroacetamide (51)

Title compound was prepared in a manner substantially similar to procedure mentioned in General Procedure B to get N-(3-{2-[(3-chloro-1-methyl-1H-pyrazol-4-yl)amino]-8-oxo-7-phenyl-5H,6H,7H,8H,9H-pyrimido[4,5-d][1,3]diazepin-9-yl}phenyl)-2,2,2-trifluoroacetamide (51) as off white solid. LCMS [M+H]⁺ 461.2

### Step 6: Synthesis of N-(3-{2-[(3-chloro-1-methyl-1H-pyrazol-4-yl)amino]-8-oxo-7-phenyl-5H,6H,7H,8H,9H-pyrimido[4,5-d][1,3]diazepin-9-yl}phenyl)prop-2-enamide (Compound 29)

Title compound was prepared in a manner substantially similar to procedure mentioned in General Procedure D, to get N-(3-{2-[(3-chloro-1-methyl-1H-pyrazol-4-yl)amino]-8-oxo-7-phenyl-5H,6H,7H,8H,9H-pyrimido[4,5-d][1,3]diazepin-9-yl}phenyl)prop-2-enamide (Compound 29) as a white solid after prep-HPLC purification (15 mg, Yield: 16%). ¹H NMR (400 MHz, DMSO d6) δ 10.24 (s, 1H), 8.43 (s, 1H), 8.21 (s, 1H), 7.80 (d, J=7.6 Hz, 1H), 7.69 (s, 1H), 7.43-7.33 (m, 5H), 7.31-7.24 (m, 1H), 7.12 (d, J=7.6 Hz, 1H), 6.44-7.37 (m, 2H), 6.25-6.21 (m, 1H), 5.73 (d, J=10.0 Hz, 1H), 4.09 (s, 2H), 3.50 (s, 3H), 3.03 (s, 2H); LCMS [M+H]⁺ 515.3.

The following compounds were prepared using the procedures described above:

| Cmpd | | Synthesis | LCMS | ¹H-NMR (400 |
|---|---|---|---|---|
| No. | Structure | Method | [M + H]⁺ | MHz, DMSO-d₆) |
| 30 | 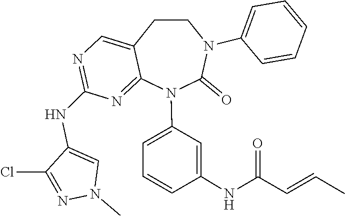 [SMILES: C/C=C/C(=O)Nc1cccc(N2C(=O)N(c3ccccc3)CCc3cnc(Nc4cn(C)nc4Cl)nc32)c1] | C | 529.3 | δ 10.06 (s, 1H), 8.48 (s, 1H), 8.22 (s, 1H), 7.76 (d, J = 8.4 Hz, 1H), 7.69 (s, 1H), 7.41-7.32 (m, 5H), 7.25 (t, J = 7.6 Hz, 1H), 7.09 (d, J = 7.6 Hz, 1H), 6.82-6.73 (m, 1H), 6.42 (bs, 1H), 6.12-6.08 (m, 1H), 4.10 (t, J = 4.4 Hz, 2H), 3.50 (s, 3H), 3.04 (t, J = 4.8 |
| | | | | Hz, 2H), 1.85 |
| | | | | (d, J = 6.8 Hz, 3H) |
| 31 | 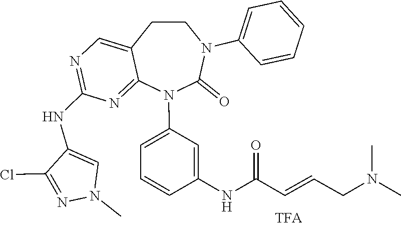 [SMILES: CN(C)C/C=C/C(=O)Nc1cccc(N2C(=O)N(c3ccccc3)CCc3cnc(Nc4cn(C)nc4Cl)nc32)c1] | C | 572.4 | δ 10.42 (s, 1H), 9.80 (s, 1H), 8.50 (s, 1H), 8.23 (s, 1H) 7.80 (d, J = 8.0 Hz, 1H), 7.70 (s, 1H), 7.47-7.38 (m, 3H), 7.34-7.32 (m, 2H), 7.24-7.20 (m, 1H), 7.16 (s, 1H), 6.95 (s, 1H), 6.76- 6.68 (m, 1H), 6.45-6.41 (m, 1H), 4.10 (s, 2H), 3.93 (s, 2H), 3.51 (s, 3H |
| | | | | merged with DMSO |
| | | | | peak), 3.05 (bs, |
| | | | | 2H), 2.78 (s, 6H). |
| 32 | 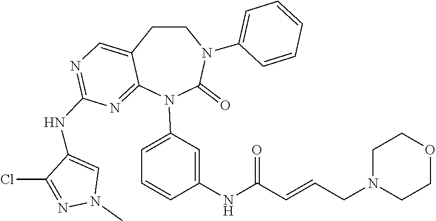 [SMILES: Cn1cc(Nc2ncc3c(n2)N(c2cccc(NC(=O)/C=C/CN4CCOCC4)c2)C(=O)N(c2ccccc2)CC3)c(Cl)n1] | C | 614.4 | δ 10.18 (s, 1H), 8.48 (s, 1H), 8.24 (s, 1H), 7.77-7.72 (m, 2H), 7.44-7.25 (m, 6H), 7.12 (d, J = 7.6 Hz, 1H), 6.76- 6.69 (m, 1H), 6.44 (bs, 1H), 6.29- 6.25 (m, 1H), 4.11 (s, 2H), 3.59 (s, 4H), 3.52 (s, 3H), 3.12-3.05 (m, 4H), 2.38 (s, 4H) |
| 33 | 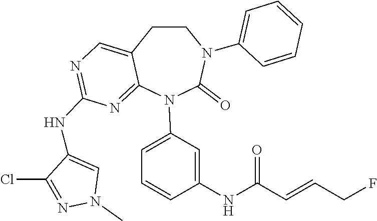 [SMILES: Cn1cc(Nc2ncc3c(n2)N(c2cccc(NC(=O)/C=C/CF)c2)C(=O)N(c2ccccc2)CC3)c(Cl)n1] | C | 547.3 | δ 10.34 (s, 1H), 8.43 (s, 1H), 8.22 (s, 1H), 7.76-7.72 (m, 2H), 7.43-7.23 (m, 6H), 7.19 (d, J = 7.6 Hz, 1H), 6.90- 6.80 (m, 1H), 6.44 (bs, 1H), 6.38- 6.34 (m, 1H), 5.15 (d, J = 48.0 Hz, 2H) 4.09 (s, 2H), 3.50 (s, 3H), 3.04 (s, 2H) |
| 34 | 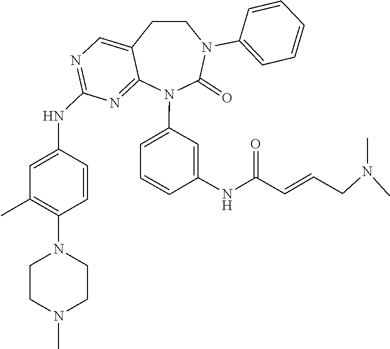 | C | 646.6 | δ 10.46 (s, 1H), 10.18 (s, 1H), 9.91 (s, 1H), 9.20 (s, 1H), 8.24 (s, 1H), 7.94 (d, J = 8.0 Hz, 1H), 7.59 (s, 1H), 7.39- 7.23 (m, 4H), 7.14 (d, J = 8.0 Hz, 1H), 6.91 (s, 1H), 6.81-6.73 (m, 2H), 6.48 (dd, J = 8.4 Hz, 2H), 4.08 (s, 2H), 3.88 (d, J = 6.8 Hz, 2H), 3.43 (d, J = 12.4 Hz, 2H), 3.17-2.97 (m, 7H), 2.84 (s, 4H), 2.75 (s, 6H), 2.06 (s, 3H) |
| 35 | 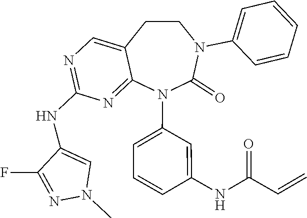 [SMILES: C=CC(=O)Nc1cccc(N2C(=O)N(c3ccccc3)CCc3cnc(Nc4cn(C)nc4F)nc32)c1] | E | 499.3 | δ 10.26 (s, 1H), 8.20 (s, 1H), 7.78 (d, J = 7.2 Hz, 1H), 7.71 (s, 1H), 7.42- 7.32 (m, 5H), 7.27- 7.25 (m, 1H), 7.12 (d, J = 7.6 Hz, 1H), 6.45-6.39 (m, 2H), 6.24 (d, J = 17.2 Hz, 2H), 5.74 (d, J = 9.6 Hz, 1H), 4.09 (s, 2H), 3.41 (s, 3H), 3.03 (s, 2H) |
| 36 | 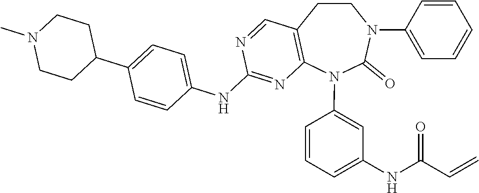 [SMILES: C=CC(=O)Nc1cccc(N2C(=O)N(c3ccccc3)CCc3cnc(Nc4ccc(C5CCN(C)CC5)cc4)nc32)c1] | E | 574.0 | δ 10.24 (s, 1H), 9.31-9.29 (m, 2H), 8.24 (s, 1H), 8.05 (d, J = 8.0 Hz, 2H), 7.52 (s, 1H), 7.40- 7.12 (m, 5H), 7.11 (d, J = 8.0 Hz, 1H), 6.94 (d, J = 8.0 Hz, 2H), 6.77 (d, J = 8.0 Hz, 2H), 6.39- 6.24 (m, 1H), 5.73 (d, J = 8.0 Hz, 1H), 4.13 (s, 2H), |
| | | | | 3.03 (s, 4H), 2.80 (s, |
| | | | | 2H), 2.48 (bs, 4H |
| | | | | merged with DMSO |
| | | | | peak), 1.88-1.68 (m, 4H) |
| 37 | 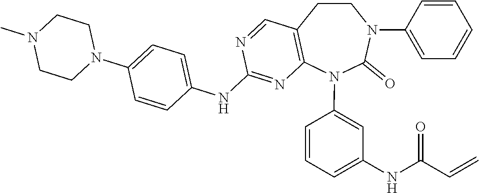 [SMILES: C=CC(=O)Nc1cccc(N2C(=O)N(c3ccccc3)CCc3cnc(Nc4ccc(N5CCN(C)CC5)cc4)nc32)c1] | E | 575.0 | δ 10.28 (s, 1H), 9.50 (s, 1H), 9.17 (s, 1H), 8.21 (s, 1H), 8.03 (d, J = 4.0 Hz, 1H), 7.56 (s, 1H), 7.33-7.40 (m, 4H), 7.26-7.28 (m, 1H), 7.08 (d, J = 8.0 Hz, 1H), 6.93 (d, J = 8.0 Hz, 2H), 6.56 (d, J = 8.0 Hz, 2H), 6.41-6.45 (m, 1H), 6.24-6.29 (m, |
| | | | | 1H), 5.74 (d, J = |
| | | | | 12.0 Hz, 1H), 4.13 |
| | | | | (s, 2H), 3.36-3.58 |
| | | | | (m, 4H), 3.03-3.13 |
| | | | | (m, 4H), 2.66- |
| | | | | 2.86 (m, 5H) |
| 38 | 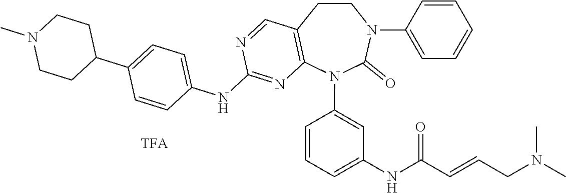 | C | 631.0 | δ 10.42 (s, 1H), 9.83 (s, 1H), 9.40- 9.31 (m, 2H), 8.25 (s, 2H), 7.92 (d, J = 7.6 Hz, 1H), 7.60 (s, 2H), 7.43-7.34 (m, 4H), 7.30 (d, J = 11.6 Hz, 1H), 6.98-6.63 (m, 4H), 6.41 (d, J = 15.2 Hz, 1H), 4.13 (s, 2H), 3.92 (s, 2H), 3.4 (bs, 2H merged with |
| | | | | DMSO peak), |
| | | | | 3.15 (bs, 1H), 3.03 |
| | | | | (bs, 4H), 2.80- |
| | | | | 2.76 (m, 9H), |
| | | | | 1.89-1.63 (m, 4H) |
| 39 | 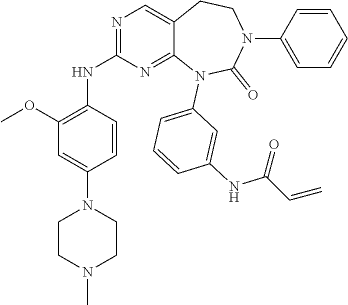 [SMILES: C=CC(=O)Nc1cccc(N2C(=O)N(c3ccccc3)CCc3cnc(Nc4ccc(N5CCN(C)CC5)cc4OC)nc32)c1] | D | 605.5 | δ 10.25 (s, 1H), 8.19 (s, 1H), 7.96 (d, J = 8.0 Hz, 1H), 7.64 (s, 1H), 7.40- 7.33 (m, 6H), 7.27-7.25 (m, 1H), 7.06 (d, J = 7.6 Hz, 1H), 6.86 (d, J = 8.4 Hz, 1H), 6.48- 6.38 (m, 2H), 6.26- 6.21 (m, 1H), 5.97 (d, J = 8.8 Hz, 1H), 5.73 (d, J = 10.0 Hz, 1H), 4.12 (s, 2H), 3.76 (s, 3H), 3.03-2.97 (m, 6H), 2.41 (bs, 4H), 2.21 (s, 3H). |
| 40 | 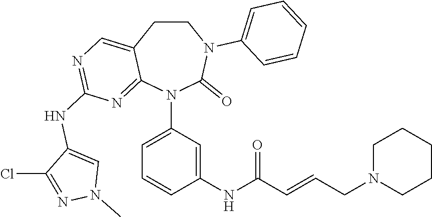 [SMILES: Cn1cc(Nc2ncc3c(n2)N(c2cccc(NC(=O)/C=C/CN4CCCCC4)c2)C(=O)N(c2ccccc2)CC3)c(Cl)n1] | C | 612.4 | δ 10.16 (s, 1H), 8.48 (s, 1H), 8.22 (s, 1H), 7.74 (t, J = 7.6 Hz, 2H), 7.39- 7.25 (m, 6H), 7.11 (d, J = 8.0 Hz, 1H), 6.71 (d, J = 14.0 Hz, 1H), 6.22 (d, J = 14.8 Hz, 2H), 4.10 (s, 2H), 3.50 (s, 3H), 3.05 (bs, 4H), 2.32 (bs, 4H), 1.49 (bs, 4H), 1.37 (bs, 2H) |
| 41 | 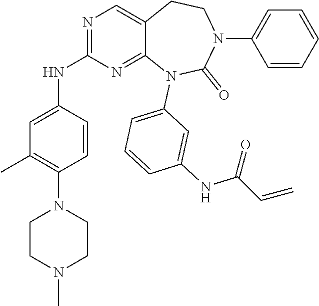 [SMILES: C=CC(=O)Nc1cccc(N2C(=O)N(c3ccccc3)CCc3cnc(Nc4ccc(N5CCN(C)CC5)c(C)c4)nc32)c1] | E | 587.4 [M − H]⁻ | δ 10.24 (s, 1H), 9.09 (s, 1H), 8.21 (s, 1H), 7.92 (d, J = 8.0 Hz, 2H), 7.67 (s, 1H), 7.39-7.32 (m, 4H), 7.26-7.25 (m, 1H), 7.07 (d, J = 8.0 Hz, 1H), 6.84 (s, 1H), 6.76 (d, J = 8.8 Hz, 1H), 6.50- 6.37 (m, 2H), 6.23 (d, J = 16.8 Hz, 1H), 5.72 (d, J = 10.0 Hz, 1H), 4.11 (bs, 2H), 3.03 (bs, 2H), 2.65 (bs, 4H), 2.40 (bs, 4H), 2.21 (s, 3H), 2.04 (s, 3H) |
| 42 | 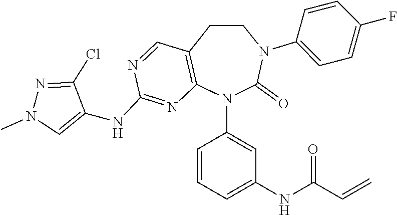 [SMILES: C=CC(=O)Nc1cccc(N2C(=O)N(c3ccc(F)cc3)CCc3cnc(Nc4cn(C)nc4Cl)nc32)c1] | E | 533.3 | δ 10.26 (s, 1H), 8.48 (s, 1H), 8.22 (s, 1H), 7.76 (d, J = 8.0 Hz, 1H), 7.70 (s, 1H), 7.44-7.37 (m, 3H), 7.22 (d, J = 8.4 Hz, 2H), 7.11 (d, J = 8.0 Hz, 1H), 6.44-6.38 (m, 2H), 6.23 (d, J = 17.2 Hz, 1H), 5.74 (d, J = 10.4 Hz, 1H), 4.06 (bs, 2H), 3.50 (s, 3H), 3.04 (bs, 2H). |

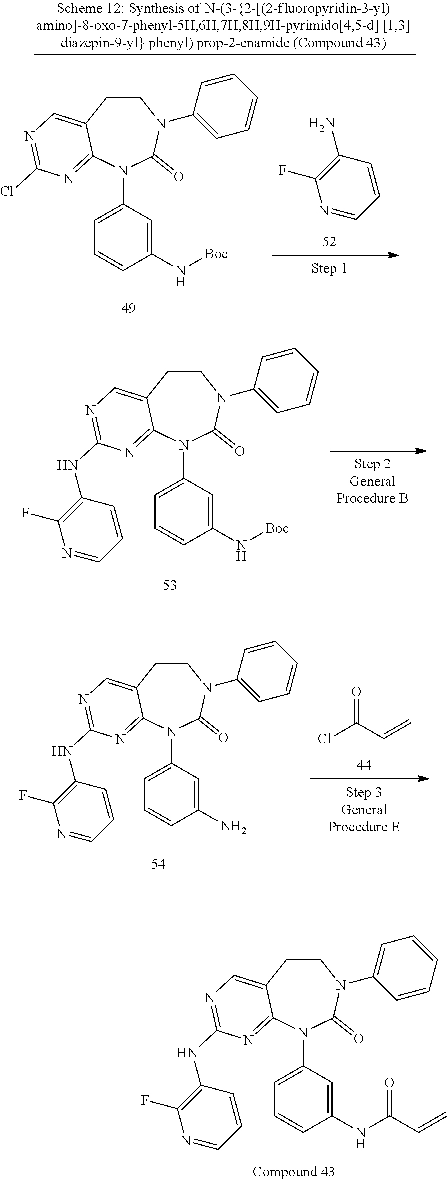

### Step 1: Synthesis of tert-butyl N-(3-{2-[(2-fluoropyridin-3-yl) amino]-8-oxo-7-phenyl-5H,6H,7H,8H,9H-pyrimido[4,5-d][1,3]diazepin-9-yl}phenyl) carbamate (53)

To a stirred solution of tert-butyl N-(3-{2-chloro-8-oxo-7-phenyl-5H,6H,7H,8H,9H-pyrimido[4,5-d][1,3]diazepin-9-yl}phenyl)carbamate (49) (0.5 g, 1.07 mmol) in 1,4-dioxane (10.0 mL) were added 2-fluoropyridin-3-amine (52) (0.15 g, 1.29 mmol), potassium carbonate (0.45 g, 3.22 mmol), RuPhos (0.1 g, 0.22 mmol) and the reaction mixture was purged with argon for 5 minutes. Then tris(dibenzylideneacetone)dipalladium(0) (0.1 g, 0.11 mmol) was added and the reaction mixture was irradiated at 130° C. for 2 hours in microwave. Progress of the reaction was monitored by TLC and LCMS. After completion of reaction, the reaction mixture was diluted with water (20 mL) and extracted with ethyl acetate (3×20 mL). The combined organic layer was dried over anhydrous sodium sulphate and evaporated under the reduced pressure. The crude product was purified by using combiflash purifier and was eluted with 50% ethyl acetate in hexane as an eluent to afford tert-butyl N-(3-{12-[(2-fluoropyridin-3-yl)amino]-8-oxo-7-phenyl-5H,6H,7H,8H,9H-pyrimido[4,5-d][1,3]diazepin-9-yl}phenyl)carbamate (53) (0.53 g, 0.98 mmol) as pale yellow solid. LCMS: [M+H]⁺ 542.3

### Step 2: Synthesis of 9-(3-aminophenyl)-2-[(2-fluoropyridin-3-yl) amino]-7-phenyl-5H,6H,7H,8H,9H-pyrimido[4,5-d][1,3]diazepin-8-one (54)

Title compound was prepared in a manner substantially similar to procedure mentioned in General Procedure B to get 9-(3-aminophenyl)-2-[(2-fluoropyridin-3-yl)amino]-7-phenyl-5H,6H,7H,8H,9H-pyrimido[4,5-d][1,3]diazepin-8-one (54) (0.43 g, 1 mmol) as pale yellow gum. LCMS: [M+H]⁺ 442.2

### Step 3: Synthesis of N-(3-{2-[(2-fluoropyridin-3-yl) amino]-8-oxo-7-phenyl-5H,6H,7H,8H,9H-pyrimido[4,5-d][1,3]diazepin-9-yl}phenyl) prop-2-enamide (Compound 43)

Title compound was prepared in a manner substantially similar to procedure mentioned in General Procedure E, to get N-(3-{12-[(2-fluoropyridin-3-yl)amino]-8-oxo-7-phenyl-5H,6H,7H,8H,9H-pyrimido[4,5-d][1,3]diazepin-9-yl}phenyl)prop-2-enamide (Compound 43) as off white solid. ¹H NMR (400 MHz, DMSO-d6): δ 10.23 (s, 1H), 8.88 (s, 1H), 8.31 (s, 1H), 7.76 (d, J=8.0 Hz, 2H), 7.70 (s, 1H), 7.58-7.27 (m, 7H), 7.10 (d, J=8.0 Hz, 1H), 6.73 (s, 1H), 6.43-6.37 (m, 1H), 6.23 (d, J=16.4 Hz, 1H), 5.73 (d, J=9.6 Hz, 1H), 4.13 (s, 2H), 3.07 (s, 2H); LCMS [M+H]⁺ 496.3

The following compounds were prepared using the procedures described above:

| Cmpd | | Synthesis | LCMS | |
|---|---|---|---|---|
| No. | Structure | Method | [M + H]⁺ | ¹H-NMR (400 MHz, DMSO-d₆) |
| 44 | 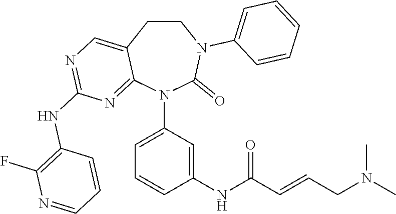 [SMILES: CN(C)C/C=C/C(=O)Nc1cccc(N2C(=O)N(c3ccccc3)CCc3cnc(Nc4cccnc4F)nc32)c1] | C | 553.3 | δ 10.14 (s, 1H), 8.87 (s, 1H), 8.33 (s, 1H), 7.73 (d, J = 8.4 Hz, 2H), 7.60 (d, J = 3.6 Hz, 1H), 7.45-7.36 (m, 6H), 7.28 (t, J = 7.2 Hz, 1H), 7.09 (d, J = 8.0 Hz, 1H), 6.75-6.69 (m, 2H), 6.24 (d, J = 15.2 Hz, 1H), 4.14 (s, 2H), 3.09- 3.03 (m, 4H), 2.16 (s, 6H). |

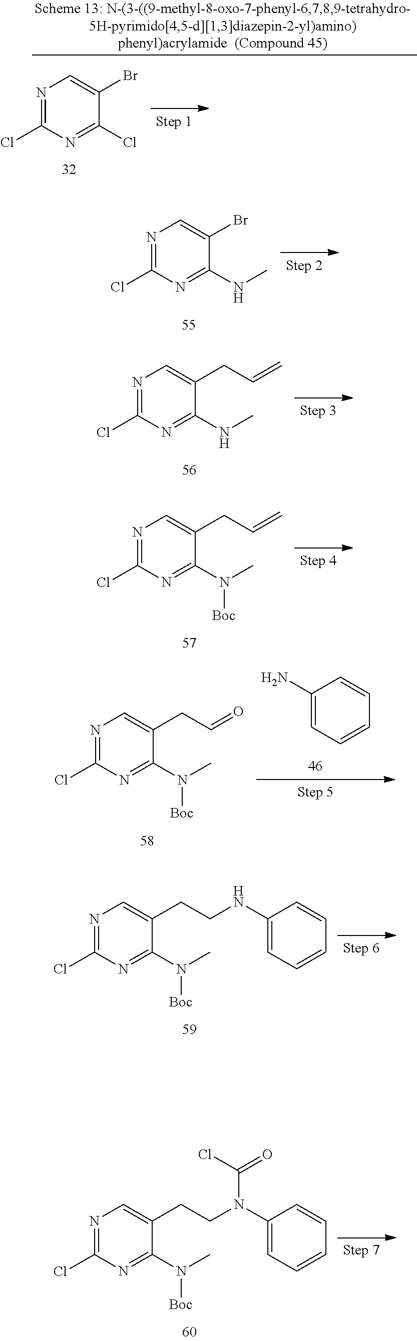

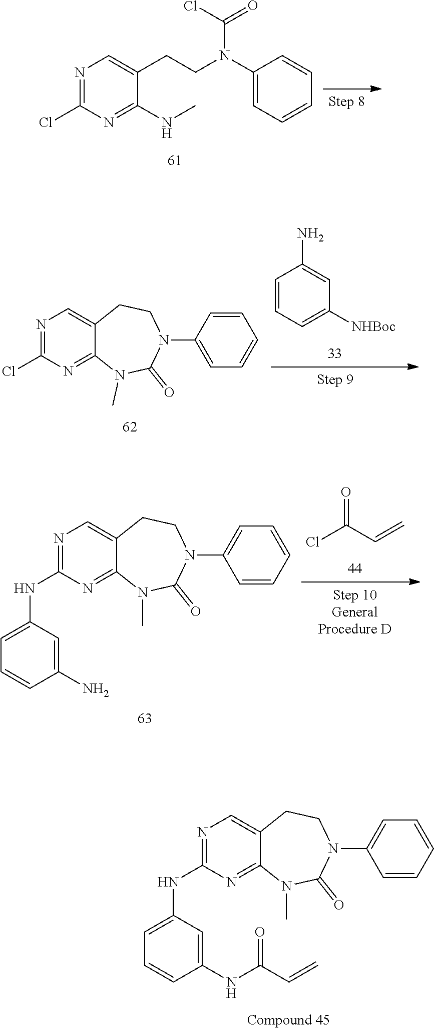

### Step 1: Synthesis of 5-bromo-2-chloro-N-methylpyrimidin-4-amine (55)

To a stirred solution of 5-bromo-2,4-dichloropyrimidine (32) (40.0 g, 176 mmol) in methanol (300 mL) at 0° C. was added methylamine (21.9 mL, 43.9 mmol, 2M in THF) under nitrogen atmosphere and the reaction mixture was stirred at room temperature for 4 hours. The reaction was monitored by TLC, after completion of starting material, the reaction mixture was concentrated under reduced pressure. The crude product was diluted with water (100 mL) and the precipitated solid was filtered, dried under vacuum to get 5-bromo-2-chloro-N-methylpyrimidin-4-amine (55) (38.0 g, Yield: 97.31%). LCMS: [M+H]⁺ 221.96.

### Step 2: Synthesis of 5-allyl-2-chloro-N-methylpyrimidin-4-amine (56)

To a solution of 5-bromo-2-chloro-N-methylpyrimidin-4-amine (55) (38.0 g, 171 mmol) in N,N-dimethylformamide (150 ml) was added allyltributylstannane (4.11 g, 12.4 mmol) and tetrakis(triphenylphosphine)palladium(0) (1.19 g, 1.03 mmol) under nitrogen atmosphere. Then the reaction mixture was heated at 100° C. for 16 hours. After completion of reaction mixture (TLC monitoring), the reaction mixture was cooled to room temperature, diluted with water (200 mL) and extracted with ethyl acetate (200 mL×3). The combined organic layer was washed with brine (200 mL), dried over anhydrous sodium sulfate and concentrated under reduced pressure. The crude product was purified by column chromatography and was eluted with 8% ethyl acetate in hexane to get 2-chloro-N-methyl-5-(prop-2-en-1-yl)pyrimidin-4-amine (56) (11.0 g, Yield: 35%). LCMS [M+H]⁺ 183.90.

### Step 3: Synthesis of tert-butyl (5-allyl-2-chloropyrimidin-4-yl)(methyl)carbamate (57)

To a solution of 2-chloro-N-methyl-5-(prop-2-en-1-yl)pyrimidin-4-amine (56) (11.0 g, 59.9 mmol) in tetrahydrofuran (50.0 mL) was added triethylamine (16.7 mL, 120 mmol) and di-tert-butyl dicarbonate (19.5 g, 89.8 mmol) at room temperature under nitrogen atmosphere. The reaction mixture was stirred at room temperature for 32 hours. After completion of reaction (TLC monitoring), the reaction mixture was diluted with water (200 mL) and extracted with ethyl acetate (200 mL×2). The combined organic layer was washed with water (200 mL), brine (200 mL), dried over anhydrous sodium sulphate, filtered and concentrated under reduced pressure. The crude product was purified by combiflash purifier and was eluted with 20% ethyl acetate in hexane to get tert-butyl N-[2-chloro-5-(prop-2-en-1-yl)pyrimidin-4-yl]-N-methylcarbamate (57) (5.40 g, Yield: 32.0%), LCMS [M+H]⁺ 283.98.

### Step 4: Synthesis of tert-butyl (2-chloro-5-(2-oxoethyl)pyrimidin-4-yl)(methyl)carbamate (58)

To a solution of tert-butyl N-[2-chloro-5-(prop-2-en-1-yl)pyrimidin-4-yl]-N-methylcarbamate (57) (5.00 g, 17.6 mmol) in ethyl acetate (50.0 mL) at −78° C. was purged ozone gas for 30 minutes. The reaction mixture was stirred at same temperature for 4 hours. The progress of reaction was monitored by TLC, after completion of reaction, dimethyl sulphide (0.876 g, 14.1 mmol) was added and stirred at room temperature for 1 hour. The reaction mixture was diluted with water (200 mL) and extracted with ethyl acetate (100 mL×2). The combined organic layer was washed with brine (200 mL), dried over anhydrous sodium sulphate, filtered and concentrated under reduced pressure to get tert-butyl N-[2-chloro-5-(2-oxoethyl)pyrimidin-4-yl]-N-methylcarbamate (58) (4.0 g, Yield: 79%). LCMS [M+H]⁺ 285.86.

### Step 5: Synthesis of tert-butyl (2-chloro-5-(2-(phenylamino)ethyl)pyrimidin-4-yl)(methyl)carbamate (59)

To an ice-cold solution of tert-butyl N-[2-chloro-5-(2-oxoethyl)pyrimidin-4-yl]-N-methylcarbamate (58) (4.0 g, 14.0 mmol) in 1,2-dichloroethane (50.0 mL) was added aniline (46) (2.5 mL, 27.98 mmol) and acetic acid (0.081 mL, 1.4 mmol). Then sodium borohydride (1.55 g, 42 mmol) was added and the reaction mixture was stirred at room temperature for 16 hours. After completion of reaction (monitored by TLC), the reaction mixture was diluted with water (100 mL) and extracted with ethyl acetate (200 mL×2). The combined organic layer was washed with brine (200 mL), dried over anhydrous sodium sulphate, filtered and concentrated under reduced pressure. The crude product was purified by combiflash purifier and was eluted with 40% ethyl acetate in hexane to get tert-butyl N-{2-chloro-5-[2-(phenylamino)ethyl]pyrimidin-4-yl}-N-methylcarbamate (59) (2.10 g, Yield: 41.66%). LCMS [M+H]⁺ 363.23.

### Step 6: Synthesis of Tert-butyl (2-chloro-5-(2-((chlorocarbonyl)(phenyl)amino)ethyl)pyrimidin-4-yl)(methyl)carbamate (60)

To a solution of tert-butyl N-{2-chloro-5-[2-(phenylamino)ethyl]pyrimidin-4-yl}-N-methylcarbamate (59) (2.10 g, 5.79 mmol) in acetonitrile (20.0 mL) was added N,N-diisopropylethylamine (4.12 mL, 23.16 mmol) and triphosgene (0.45 g, 2.31 mmol) at room temperature. The resultant reaction mixture was heated at 100° C. for 4 hours. After completion of reaction (TLC monitoring), the reaction mixture was dilute with water (100 mL) and extracted with ethyl acetate (100 mL×2). The combined organic layer was washed with brine (100 mL), dried over anhydrous sodium sulphate, filtered and concentrated under reduced pressure. The crude product was purified by combiflash purifier and was eluted with 54% ethyl acetate in hexane to get tert-butyl N-(2-chloro-5-{2-[(chlorocarbonyl)(phenyl)amino]ethyl}pyrimidin-4-yl)-N-methylcarbamate (60) (1.50 g, Yield: 61.2%). LCMS [M+H]⁺ 424.95.

### Step 7: Synthesis of (2-(2-chloro-4-(methylamino)pyrimidin-5-yl)ethyl)(phenyl)carbamic chloride (61)

To an ice-cold solution of tert-butyl N-(2-chloro-5-{2-[(chlorocarbonyl)(phenyl)amino]ethyl}pyrimidin-4-yl)-N-methylcarbamate (60) (1.50 g, 4.61 mmol) in dichloromethane (15 mL) was added trifluoroacetic acid (2.3 mL, 23.05 mmol) under nitrogen atmosphere. The reaction mixture was stirred at room temperature for 2 hours. After completion of reaction (TLC monitoring), the reaction mixture was concentrated under reduced pressure to get N-{2-[2-chloro-4-(methylamino)pyrimidin-5-yl]ethyl}-N-phenylcarbamoyl chloride (61) (1.15 g, crude). LCMS [M+H]⁺ 324.96.

### Step 8: Synthesis of 2-chloro-9-methyl-7-phenyl-5,6,7,9-tetrahydro-8H-pyrimido[4,5-d][1,3]diazepin-8-one (62)

To a solution of N-{2-[2-chloro-4-(methylamino)pyrimidin-5-yl]ethyl}-N-phenylcarbamoyl chloride (61) (1.15 g, 3.53 mmol) in acetonitrile (15.0 mL) was added triethylamine (1.4 mL, 14.12 mmol) and N,N-dimethylpyridin-4-amine (0.259 g, 2.12 mmol). The reaction mixture was heated at 100° C. for 4 hours. After completion of reaction (TLC monitoring), the reaction mixture was diluted with water (50 mL) and extracted with ethyl acetate (100 mL×2). The combined organic layer was washed with brine (50 mL), dried over anhydrous sodium sulphate, filtered and concentrated under reduced pressure. The crude product was purified by combiflash purifier and was eluted with 60% ethyl acetate in hexane to get 2-chloro-9-methyl-7-phenyl-5H,6H,7H,8H,9H-pyrimido[4,5-d][1,3]diazepin-8-one (62) (0.8 g, Yield: 60.07%). LCMS: [M+H]⁺ 288.86

### Step 9: Synthesis of 2-((3-aminophenyl)amino)-9-methyl-7-phenyl-5,6,7,9-tetrahydro-8H-pyrimido[4,5-d][1,3]diazepin-8-one (63)

To an ice-cold solution of 2-chloro-9-methyl-7-phenyl-5,6,7,9-tetrahydro-8H-pyrimido[4,5-d][1,3]diazepin-8-one (62) (0.55 g, 1.90 mmol) in propan-2-ol (8.0 mL) was added trifluoroacetic acid (0.28 mL, 3.80 mmol) and tert-butyl (3-aminophenyl)carbamate (33) (0.476 g, 2.28 mmol). The reaction mixture was heated at 100° C. for 16 hours. After completion of reaction (TLC monitoring), the reaction mixture was concentrated under reduced pressure. The crude product was purified by combiflash purifier and was eluted with 1% methanol in dichloromethane to get 2-((3-aminophenyl)amino)-9-methyl-7-phenyl-5,6,7,9-tetrahydro-8H-pyrimido[4,5-d][1,3]diazepin-8-one (63) (0.4 g, Yield: 45%). LCMS: [M+H]⁺ 361.11.

### Step 10: Synthesis of N-(3-((9-methyl-8-oxo-7-phenyl-6,7,8,9-tetrahydro-5H-pyrimido[4,5-d][1,3]diazepin-2-yl)amino)phenyl)acrylamide (Compound 45)

Title compound was prepared in a manner substantially similar to procedure mentioned in General Procedure D, to get N-(3-((9-methyl-8-oxo-7-phenyl-6,7,8,9-tetrahydro-5H-pyrimido[4,5-d][1,3]diazepin-2-yl)amino)phenyl)acrylamide (Compound 45) (60 mg, Yield: 26%). ¹H NMR (400 MHz, DMSO-d6): δ 10.05 (bs, 1H), 9.45 (bs, 1H), 8.19 (s, 1H), 8.04 (s, 1H), 7.44-7.38 (m, 3H), 7.31-7.28 (m, 2H), 7.22-7.11 (m, 3H), 6.43-6.36 (m, 1H), 6.26-6.17 (m, 1H), 5.71 (d, J=10.0 Hz, 1H), 3.99-3.96 (m, 2H), 3.24 (s, 3H), 2.94-2.92 (s, 2H); LCMS [M+H]⁺ 415.20

### Example 2: Cellular Proliferation (Alamar Blue) Assays

### Cell Line Details:

1. EGFR(D770_N771insSVD) expressing Ba/F3 stable cell line
2. EGFR (A767_dupASV) expressing Ba/F3 stable cell line
3. A431 cells
4. EGFR (H773insNPH) expressing Ba/F3 stable cell line
5. HER2 (A775_G775insYVMA) expressing Ba/F3 stable cell line

### Assay Procedure:

1. Seed cells at 5000 for A431 and 15,000 cells for Ba/F3 in 100 μL/well in complete media (for A431: DMEM with 10% FBS and for Ba/F3 cells: RPMI with 10% FBS) in
96-well tissue culture plate. Leave outer wells without cells for background measurements. Incubate at 37 degree Celsius in 5% C02 humidified incubator for 16-18 hours.
2. Add 0.025 ml of 5× concentration compound dilution or DMSO control. Final compound concentration range is 10-0.0005 μM prepared in 3-fold serial dilutions. Incubate for 72 hr at 37 degree Celsius in 5% CO2 humidified incubator.
3. Add 0.0125 ml Alamar Blue™ reagent to each well with multi-channel pipette and tap gently on each side of the plate to mix. Incubate for 3 hours at 37 degree Celsius in 5% CO2 humidified incubator.
4. Read plates on fluorescence reader (Tecan Spark Control, Device: Spark, Serial #: 1801006040) at 540 nm excitation, 590 nm emission wavelength.
5. Data analysis was performed using XLfit 5.5.0.5.

Table 1 shows the activity of compounds of the present disclosure in the EGFR and HER2 cellular proliferation assays.

TABLE 1
Cellular proliferation data.

| | | A431 | A767 | D770 | NPH | YVMA |
|---|---|---|---|---|---|---|
| Cmpd | | IC₅₀ | IC₅₀ | IC₅₀ | IC₅₀ | IC₅₀ |
| No. | Structure | (nM) | (nM) | (nM) | (nM) | (nM) |
| 1 | 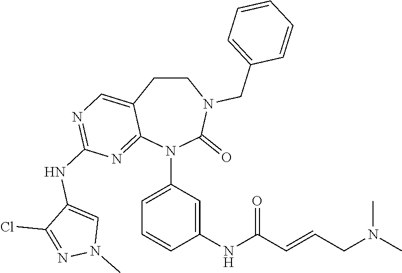 [SMILES: CN(C)C/C=C/C(=O)Nc1cccc(N2C(=O)N(Cc3ccccc3)CCc3cnc(Nc4cn(C)nc4Cl)nc32)c1] | 132 | 54 | 105 | 86 | 31 |
| 2 | 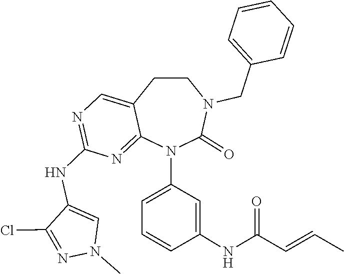 [SMILES: C/C=C/C(=O)Nc1cccc(N2C(=O)N(Cc3ccccc3)CCc3cnc(Nc4cn(C)nc4Cl)nc32)c1] | >10000 | 261 | 249 | ND | 243 |
| 3 | 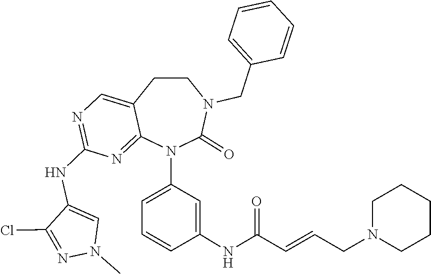 [SMILES: Cn1cc(Nc2ncc3c(n2)N(c2cccc(NC(=O)/C=C/CN4CCCCC4)c2)C(=O)N(Cc2ccccc2)CC3)c(Cl)n1] | >10000 | 42 | 48 | 108 | 31 |
| 4 | 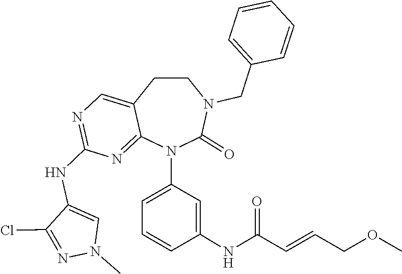 [SMILES: COC/C=C/C(=O)Nc1cccc(N2C(=O)N(Cc3ccccc3)CCc3cnc(Nc4cn(C)nc4Cl)nc32)c1] | >10000 | 104 | 98 | 103 | 125 |
| 5 | 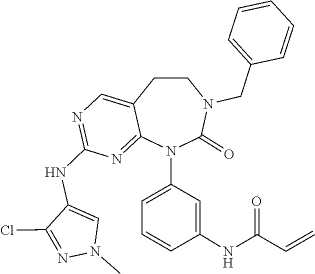 [SMILES: C=CC(=O)Nc1cccc(N2C(=O)N(Cc3ccccc3)CCc3cnc(Nc4cn(C)nc4Cl)nc32)c1] | 2 | 12 | 10 | 7 | 12 |
| 6 | 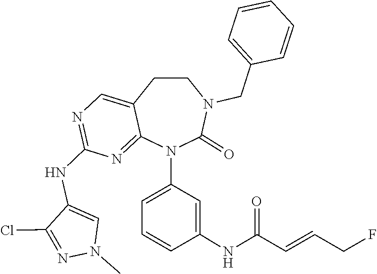 [SMILES: Cn1cc(Nc2ncc3c(n2)N(c2cccc(NC(=O)/C=C/CF)c2)C(=O)N(Cc2ccccc2)CC3)c(Cl)n1] | 8 | 9 | 12 | 32 | 10 |
| 7 | 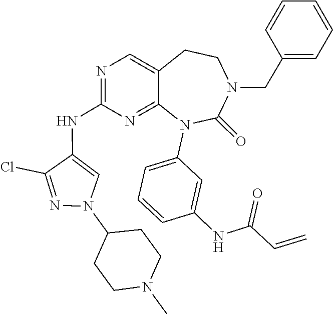 [SMILES: C=CC(=O)Nc1cccc(N2C(=O)N(Cc3ccccc3)CCc3cnc(Nc4cn(C5CCN(C)CC5)nc4Cl)nc32)c1] | 2 | 27 | 36 | 41 | 24 |
| 8 | 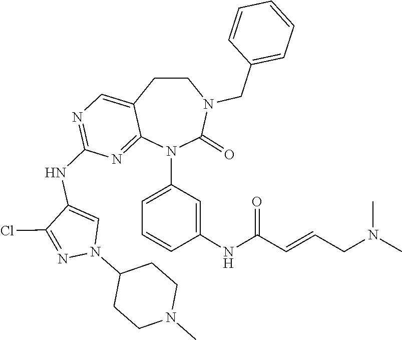 [SMILES: CN(C)C/C=C/C(=O)Nc1cccc(N2C(=O)N(Cc3ccccc3)CCc3cnc(Nc4cn(C5CCN(C)CC5)nc4Cl)nc32)c1] | 114 | 329 | 940 | ND | ND |
| 9 | 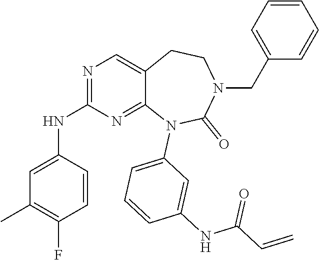 [SMILES: C=CC(=O)Nc1cccc(N2C(=O)N(Cc3ccccc3)CCc3cnc(Nc4ccc(F)c(C)c4)nc32)c1] | 35 | 46 | 45 | 95 | 28 |
| 10 | 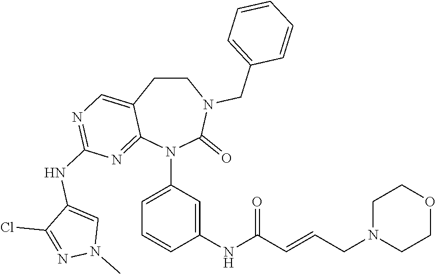 [SMILES: Cn1cc(Nc2ncc3c(n2)N(c2cccc(NC(=O)/C=C/CN4CCOCC4)c2)C(=O)N(Cc2ccccc2)CC3)c(Cl)n1] | 8 | 43 | 47 | 94 | 20 |
| 11 | 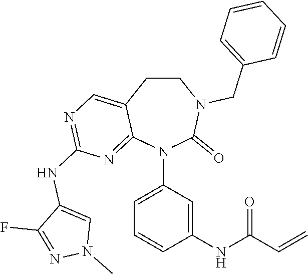 [SMILES: C=CC(=O)Nc1cccc(N2C(=O)N(Cc3ccccc3)CCc3cnc(Nc4cn(C)nc4F)nc32)c1] | 5 | 11 | 10 | 31 | 7 |
| 12 | 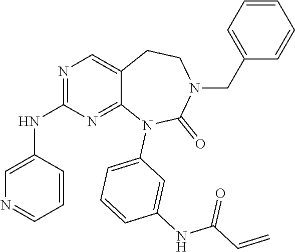 [SMILES: C=CC(=O)Nc1cccc(N2C(=O)N(Cc3ccccc3)CCc3cnc(Nc4cccnc4)nc32)c1] | >10000 | >10000 | >10000 | ND | ND |
| 13 | 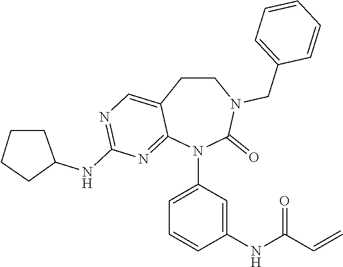 [SMILES: C=CC(=O)Nc1cccc(N2C(=O)N(Cc3ccccc3)CCc3cnc(NC4CCCC4)nc32)c1] | >10000 | 848 | 1060 | ND | 1034 |
| 14 | 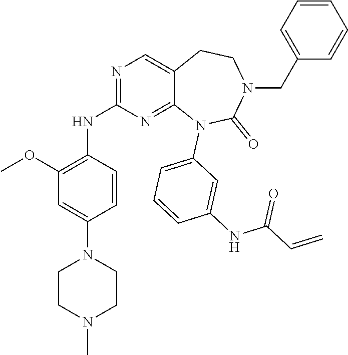 [SMILES: C=CC(=O)Nc1cccc(N2C(=O)N(Cc3ccccc3)CCc3cnc(Nc4ccc(N5CCN(C)CC5)cc4OC)nc32)c1] | 34 | 33 | 38 | 32 | 15 |
| 15 | 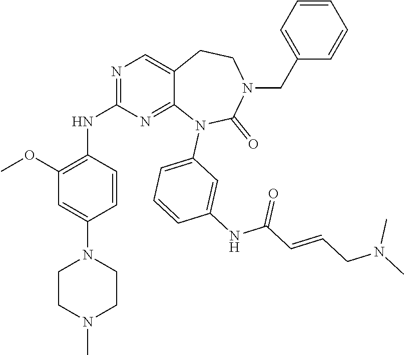 | 6019 | 348 | 959 | ND | 174 |
| 16 | 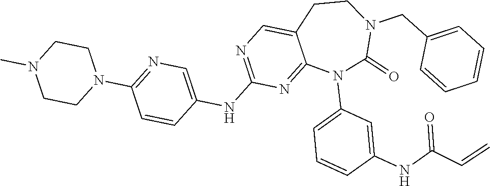 [SMILES: C=CC(=O)Nc1cccc(N2C(=O)N(Cc3ccccc3)CCc3cnc(Nc4ccc(N5CCN(C)CC5)nc4)nc32)c1] | 9 | 25 | 39 | 32 | 25 |
| 17 | 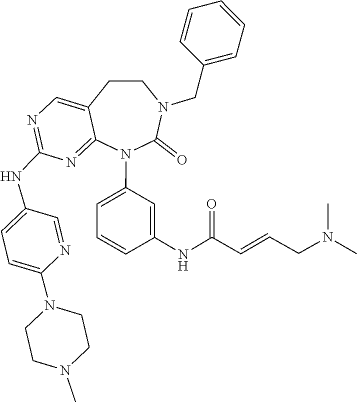 [SMILES: CN(C)C/C=C/C(=O)Nc1cccc(N2C(=O)N(Cc3ccccc3)CCc3cnc(Nc4ccc(N5CCN(C)CC5)nc4)nc32)c1] | >10000 | 1126 | 2774 | ND | 793 |
| 18 | 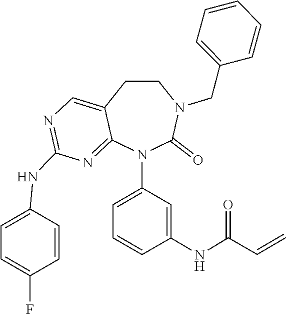 [SMILES: C=CC(=O)Nc1cccc(N2C(=O)N(Cc3ccccc3)CCc3cnc(Nc4ccc(F)cc4)nc32)c1] | 64 | 55 | 45 | 152 | 31 |
| 19 | 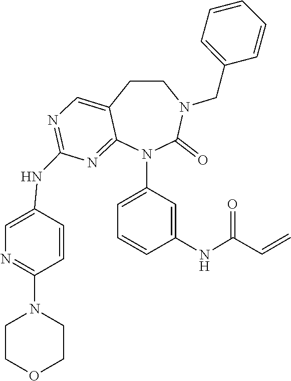 [SMILES: C=CC(=O)Nc1cccc(N2C(=O)N(Cc3ccccc3)CCc3cnc(Nc4ccc(N5CCOCC5)nc4)nc32)c1] | >10000 | 10 | 14 | 25 | 24 |
| 20 | 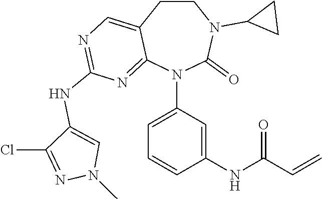 [SMILES: C=CC(=O)Nc1cccc(N2C(=O)N(C3CC3)CCc3cnc(Nc4cn(C)nc4Cl)nc32)c1] | >10000 | 225 | 320 | ND | 895 |
| 21 | 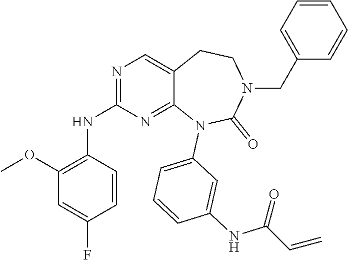 [SMILES: C=CC(=O)Nc1cccc(N2C(=O)N(Cc3ccccc3)CCc3cnc(Nc4ccc(F)cc4OC)nc32)c1] | 40 | 97 | 103 | 102 | 44 |
| 22 | 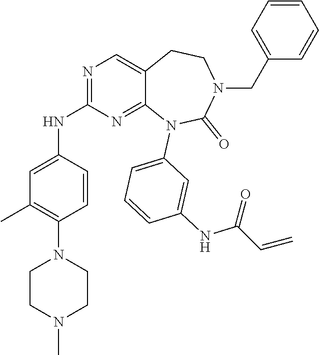 [SMILES: C=CC(=O)Nc1cccc(N2C(=O)N(Cc3ccccc3)CCc3cnc(Nc4ccc(N5CCN(C)CC5)c(C)c4)nc32)c1] | 25 | 33 | 31 | 41 | 9 |
| 23 | 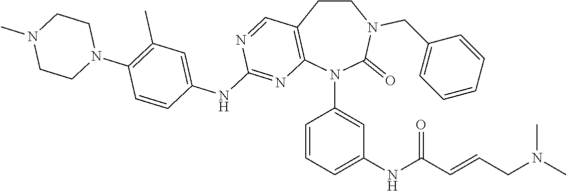 | 179 | 911 | 933 | ND | ND |
| 24 | 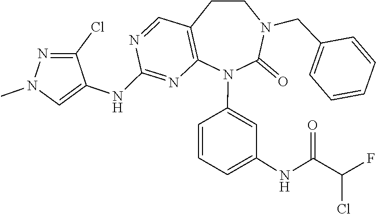 [SMILES: Cn1cc(Nc2ncc3c(n2)N(c2cccc(NC(=O)C(F)Cl)c2)C(=O)N(Cc2ccccc2)CC3)c(Cl)n1] | >10000 | 1146 | 1093 | ND | 1997 |
| 25 | 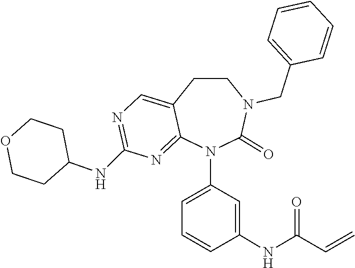 [SMILES: C=CC(=O)Nc1cccc(N2C(=O)N(Cc3ccccc3)CCc3cnc(NC4CCOCC4)nc32)c1] | >10000 | 388 | 492 | ND | ND |
| 26 | 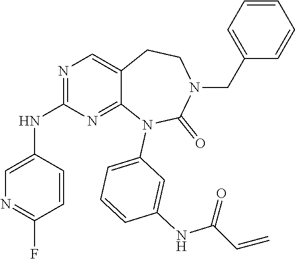 [SMILES: C=CC(=O)Nc1cccc(N2C(=O)N(Cc3ccccc3)CCc3cnc(Nc4ccc(F)nc4)nc32)c1] | 134 | 67 | 35 | 90 | 33 |
| 27 | 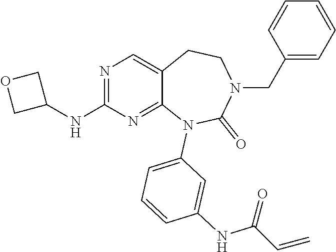 [SMILES: C=CC(=O)Nc1cccc(N2C(=O)N(Cc3ccccc3)CCc3cnc(NC4COC4)nc32)c1] | >10000 | 606 | 611 | ND | ND |
| 28 | 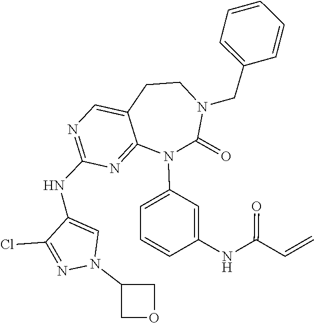 [SMILES: C=CC(=O)Nc1cccc(N2C(=O)N(Cc3ccccc3)CCc3cnc(Nc4cn(C5COC5)nc4Cl)nc32)c1] | 9 | 13 | 12 | 32 | 8 |
| 29 | 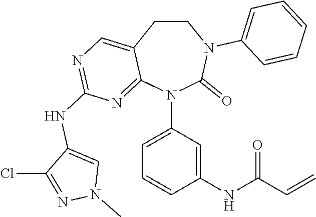 [SMILES: C=CC(=O)Nc1cccc(N2C(=O)N(c3ccccc3)CCc3cnc(Nc4cn(C)nc4Cl)nc32)c1] | 16 | 36 | 45 | 25 | 67 |
| 30 | 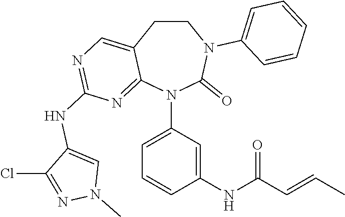 [SMILES: C/C=C/C(=O)Nc1cccc(N2C(=O)N(c3ccccc3)CCc3cnc(Nc4cn(C)nc4Cl)nc32)c1] | >10000 | 993 | 1957 | ND | 3323 |
| 31 | 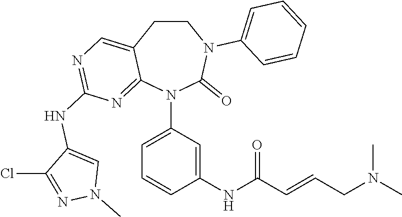 [SMILES: CN(C)C/C=C/C(=O)Nc1cccc(N2C(=O)N(c3ccccc3)CCc3cnc(Nc4cn(C)nc4Cl)nc32)c1] | >10000 | 2038 | 3949 | ND | 2420 |
| 32 | 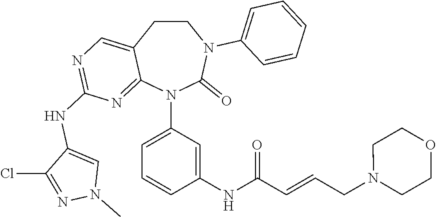 [SMILES: Cn1cc(Nc2ncc3c(n2)N(c2cccc(NC(=O)/C=C/CN4CCOCC4)c2)C(=O)N(c2ccccc2)CC3)c(Cl)n1] | >10000 | 1156 | 1359 | ND | 3282 |
| 33 | 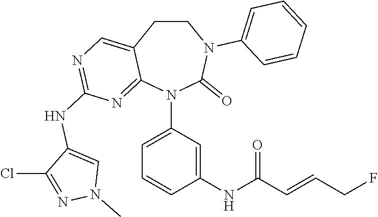 [SMILES: Cn1cc(Nc2ncc3c(n2)N(c2cccc(NC(=O)/C=C/CF)c2)C(=O)N(c2ccccc2)CC3)c(Cl)n1] | >10000 | 128 | 239 | 131 | 602 |
| 34 | 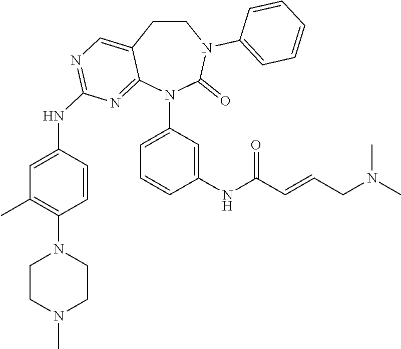 | 225 | 2794 | 3484 | ND | ND |
| 35 | 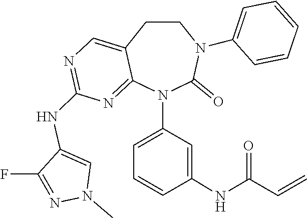 [SMILES: C=CC(=O)Nc1cccc(N2C(=O)N(c3ccccc3)CCc3cnc(Nc4cn(C)nc4F)nc32)c1] | >10000 | 117 | 122 | 138 | 1062 |
| 36 | 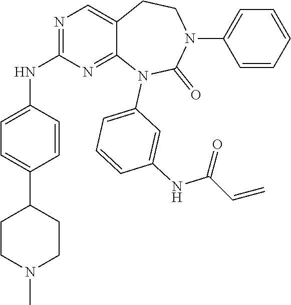 [SMILES: C=CC(=O)Nc1cccc(N2C(=O)N(c3ccccc3)CCc3cnc(Nc4ccc(C5CCN(C)CC5)cc4)nc32)c1] | 20 | 318 | 325 | ND | ND |
| 37 | 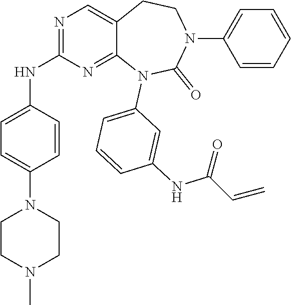 [SMILES: C=CC(=O)Nc1cccc(N2C(=O)N(c3ccccc3)CCc3cnc(Nc4ccc(N5CCN(C)CC5)cc4)nc32)c1] | 7 | 38 | 89 | 100 | 138 |
| 38 | 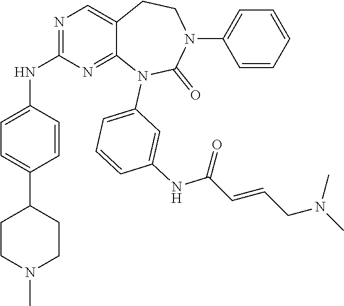 [SMILES: CN(C)C/C=C/C(=O)Nc1cccc(N2C(=O)N(c3ccccc3)CCc3cnc(Nc4ccc(C5CCN(C)CC5)cc4)nc32)c1] | 370 | 9008 | 989 | ND | ND |
| 39 | 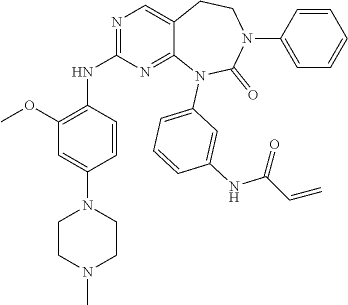 [SMILES: C=CC(=O)Nc1cccc(N2C(=O)N(c3ccccc3)CCc3cnc(Nc4ccc(N5CCN(C)CC5)cc4OC)nc32)c1] | 7 | 39 | 92 | 88 | 131 |
| 40 | 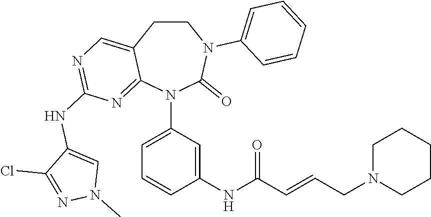 [SMILES: Cn1cc(Nc2ncc3c(n2)N(c2cccc(NC(=O)/C=C/CN4CCCCC4)c2)C(=O)N(c2ccccc2)CC3)c(Cl)n1] | >10000 | 325 | 406 | ND | 536 |
| 41 | 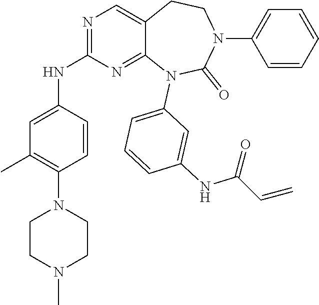 [SMILES: C=CC(=O)Nc1cccc(N2C(=O)N(c3ccccc3)CCc3cnc(Nc4ccc(N5CCN(C)CC5)c(C)c4)nc32)c1] | 102 | 32 | 41 | 27 | 29 |
| 42 | 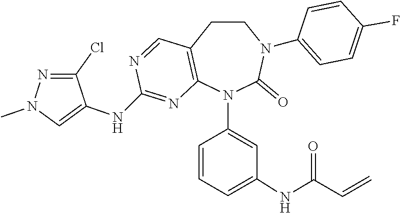 [SMILES: C=CC(=O)Nc1cccc(N2C(=O)N(c3ccc(F)cc3)CCc3cnc(Nc4cn(C)nc4Cl)nc32)c1] | 23 | 75 | 110 | 3 | 91 |
| 43 | 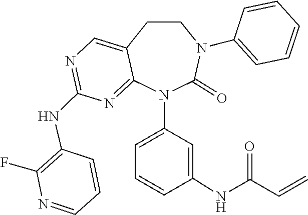 [SMILES: C=CC(=O)Nc1cccc(N2C(=O)N(c3ccccc3)CCc3cnc(Nc4cccnc4F)nc32)c1] | >10000 | 146 | 239 | 193 | 1359 |
| 44 | 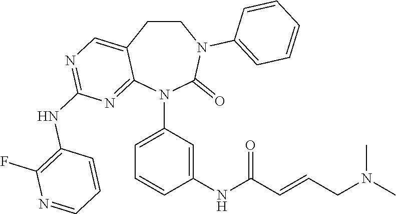 [SMILES: CN(C)C/C=C/C(=O)Nc1cccc(N2C(=O)N(c3ccccc3)CCc3cnc(Nc4cccnc4F)nc32)c1] | >10000 | 936 | 1137 | ND | 1232 |
| 45 | 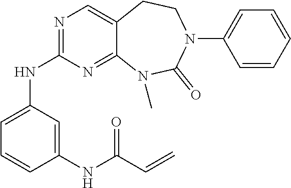 [SMILES: C=CC(=O)Nc1cccc(Nc2ncc3c(n2)N(C)C(=O)N(c2ccccc2)CC3)c1] | 1224 | >10000 | >10000 | ND | ND |
| 46 | 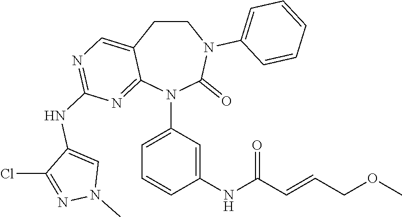 [SMILES: COC/C=C/C(=O)Nc1cccc(N2C(=O)N(c3ccccc3)CCc3cnc(Nc4cn(C)nc4Cl)nc32)c1] | >10000 | 1134 | 2766 | ND | 5951 |

ND: Not determined

1. A compound of Formula I: 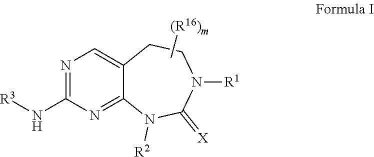
or a pharmaceutically acceptable salt thereof, wherein:
X is O or S;
R¹ is —(C(R⁴)₂)ₙR⁵, wherein R⁵ is substituted with 0, 1, or 2 R^{5′};
n is 0, 1, 2, or 3;
each R⁴ is independently hydrogen, alkyl, halo, haloalkyl, hydroxy, alkoxy, or heteroalkyl;
R⁵ is cycloalkyl, heterocycloalkyl, aryl, heteroaryl;
each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy;
each R⁶ is independently alkyl, cycloalkyl, aryl, or heteroaryl;
R² is aryl, heteroaryl, cycloalkyl, or heterocycloalkyl, wherein the aryl, heteroaryl, cycloalkyl, or heterocycloalkyl is substituted with at least one R⁷ and 0, 1, or 2 R⁸;
each R⁷ is independently 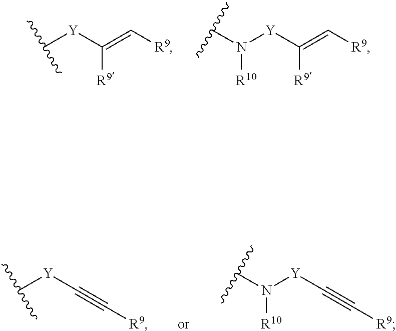
Y is —C(═O)—, —S(═O)—, or —S(═O)₂—;
R⁹ and R^{9′} are independently hydrogen, halo, alkyl, haloalkyl, cycloalkyl, heteroalkyl, or (alkyl)heterocycloalkyl;
R¹⁰ is hydrogen, alkyl, haloalkyl, or cycloalkyl;
each R⁸ is independently aryl, heteroaryl, alkyl, cycloalkyl, heterocycloalkyl, halo, heteroalkyl, haloalkyl, cyano, hydroxy, amino, —N(R¹¹)₂, —S(═O)₂alkyl, —S(═O)₂aryl, —S(═O)₂heteroaryl, or alkoxy;
each R¹¹ is independently alkyl, cycloalkyl, aryl, or heteroaryl;
R³ is alkyl, aryl, heteroaryl, cycloalkyl, or heterocycloalkyl substituted with 0, 1, 2, or 3 R¹²;
each R¹² is independently aryl, heteroaryl, alkyl, heteroalkyl, haloalkyl, halo, cyano, alkoxy, heterocycloalkyl, —N(R¹³)₂, —S(═O)₂NH₂, —S(═O)₂NMe₂, —S(═O)₂alkyl, —S(═O)₂aryl, —S(═O)₂heteroaryl, or cycloalkyl, wherein the aryl, heteroaryl, heterocycloalkyl, or cycloalkyl are each independently substituted with 0, 1, or 2 R¹⁴;
each R¹³ is independently alkyl, cycloalkyl, aryl, or heteroaryl
each R¹⁴ is independently aryl, heteroaryl, alkyl, cycloalkyl, heterocycloalkyl, halo, heteroalkyl, haloalkyl, cyano, hydroxy, amino, —N(R¹⁵)₂, —S(═O)₂alkyl, —S(═O)₂aryl, —S(═O)₂heteroaryl, or alkoxy;
each R¹⁵ is independently alkyl, cycloalkyl, aryl, or heteroaryl;
each R¹⁶ is independently aryl, heteroaryl, alkyl, heteroalkyl, haloalkyl, halo, cyano, hydroxy, amino, alkoxy, heterocycloalkyl, —N(R¹⁷)₂, —S(═O)₂NH₂, —S(═O)₂NMe₂, —S(═O)₂alkyl, —S(═O)₂aryl, —S(═O)₂heteroaryl, or cycloalkyl, wherein the aryl, heteroaryl, heterocycloalkyl, or cycloalkyl are each independently substituted with 0, 1, or 2 R¹⁸;
each R¹⁷ is independently alkyl, cycloalkyl, aryl, or heteroaryl
each R¹⁸ is independently aryl, heteroaryl, alkyl, cycloalkyl, heterocycloalkyl, halo, heteroalkyl, haloalkyl, cyano, hydroxy, amino, —N(R¹⁹)₂, —S(═O)₂alkyl, —S(═O)₂aryl, —S(═O)₂heteroaryl, or alkoxy;
each R¹⁹ is independently alkyl, cycloalkyl, aryl, or heteroaryl; and
m is 0, 1, 2, 3, or 4.

2. (canceled)

3. The compound of claim 1 or a pharmaceutically acceptable salt thereof, wherein R⁵ is cyclopropyl, phenyl, naphthyl, anthracenyl, phenanthrenyl, pyridyl, pyrimidinyl, pyrazolyl, or imidazolyl and wherein R⁵ is unsubstituted or R⁵ is substituted with 1 or 2 R^{5′}.

4. (canceled)

5. (canceled)

6. The compound of claim 3 or a pharmaceutically acceptable salt thereof, wherein each R⁴ is independently hydrogen, alkyl, halo, haloalkyl, or alkoxy.

7. (canceled)

8. (canceled)

9. (canceled)

10. The compound of claim 6 or a pharmaceutically acceptable salt thereof, wherein each R^{5′} is independently phenyl, pyrrolyl, imidazolyl, thiazolyl, isothiazolyl, oxazolyl, isoxazolyl, pyridinyl, pyrimidinyl, methyl, ethyl, tert-butyl, pyrrolidinyl, piperidinyl, piperazinyl, morpholinyl, fluoro, chloro, cyano, hydroxy, —N(R⁶)₂, methoxy, ethoxy, or trifluoromethoxy.

11. (canceled)

12. The compound of claim 10 or a pharmaceutically acceptable salt thereof, wherein each R⁶ is independently alkyl or aryl.

13-17. (canceled)

18. The compound of claim 12 or a pharmaceutically acceptable salt thereof, wherein R² is phenyl, cyclopropyl, cyclobutyl, cyclopentyl, cyclohexyl, pyrrolyl, imidazolyl, pyrazolyl, triazolyl, tetrazolyl, thiazolyl, isothiazolyl, oxazolyl, isoxazolyl, pyridinyl, pyrimidinyl, pyridazinyl, pyrazinyl, or triazinyl, wherein R² is unsubstituted or substituted with 1 or 2 R⁸.

19. (canceled)

20. The compound of claim 18 or a pharmaceutically acceptable salt thereof, wherein R⁷ is 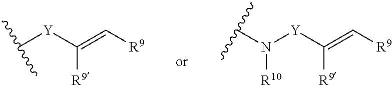
and Y is —C(═O) or —S(═O)₂.

21-23. (canceled)

24. The compound of claim 20 or a pharmaceutically acceptable salt thereof, wherein R⁹ and R^{9′} are independently hydrogen, halo, alkyl, heteroalkyl, haloalkyl, or (alkyl)heterocycloalkyl.

25. (canceled)

26. (canceled)

27. The compound claim 24 or a pharmaceutically acceptable salt thereof, wherein R¹⁰ is hydrogen, methyl, ethyl, n-propyl, iso-propyl, n-butyl, sec-butyl, tert-butyl, trifluoromethyl, or cyclopropyl.

28. (canceled)

29. (canceled)

30. The compound of claim 27 or a pharmaceutically acceptable salt thereof, wherein each R⁸ is independently methyl, ethyl, n-propyl, iso-propyl, n-butyl, iso-butyl, sec-butyl, tert-butyl, fluoro, chloro, heteroalkyl, cyano, hydroxy, amino, —N(R¹¹)₂, methoxy, ethoxy, or trifluoromethoxy.

31-32. (canceled)

33. The compound of claim 30 or a pharmaceutically acceptable salt thereof, wherein each R¹¹ is independently methyl, ethyl, n-propyl, iso-propyl, n-butyl, iso-butyl, sec-butyl, tert-butyl, phenyl, naphthyl, anthracenyl, or phenanthrenyl.

34-36. (canceled)

37. The compound of claim 33 or a pharmaceutically acceptable salt thereof, wherein R³ is phenyl, cyclopentyl, tetrahydropyranyl, oxetanyl, pyrrolyl, imidazolyl, pyrazolyl, triazolyl, tetrazolyl, indolyl, indazolyl, benzimidazolyl, azaindolyl, thiazolyl, isothiazolyl, oxazolyl, isoxazolyl, pyridinyl, pyrimidinyl, pyridazinyl, pyrazinyl, triazinyl, quinolinyl, isoquinolinyl, quinoxalinyl, quinazolinyl, cinnolinyl, or naphthyridinyl and wherein R³ is unsubstituted or substituted with at least 1 R¹².

38-45. (canceled)

46. The compound of claim 37 or a pharmaceutically acceptable salt thereof, wherein each R¹² is independently methyl, ethyl, n-propyl, iso-propyl, n-butyl, iso-butyl, sec-butyl, tert-butyl, hydroxyethyl, methoxyethyl, trifluoromethyl, trifluoroethyl, pentafluoroethyl, fluoro, chloro, cyano, methoxy, azetidinyl, oxetanyl, pyrrolidinyl, imidazolidinyl, tetrahydrofuranyl, piperidinyl, piperazinyl, tetrahydropyranyl, morpholinyl, —N(R¹³)₂, cyclopropyl, cyclobutyl, cyclopentyl, or cyclohexyl, wherein R¹² is unsubstituted or substituted 1 or 2 R¹⁴.

47-49. (canceled)

50. The compound of claim 46 or a pharmaceutically acceptable salt thereof, wherein each R¹³ is independently alkyl or cycloalkyl.

51-55. (canceled)

56. The compound of claim 51 or a pharmaceutically acceptable salt thereof, wherein each R¹⁴ is independently alkyl, cycloalkyl, heterocycloalkyl, halo, cyano, —N(R¹⁵)₂, or alkoxy.

57. (canceled)

58. (canceled)

59. The compound of claim 56 or a pharmaceutically acceptable salt thereof, wherein each R¹⁵ is independently alkyl or cycloalkyl.

60-62. (canceled)

63. The compound of claim 1 or a pharmaceutically acceptable salt thereof, wherein the compound is selected from:

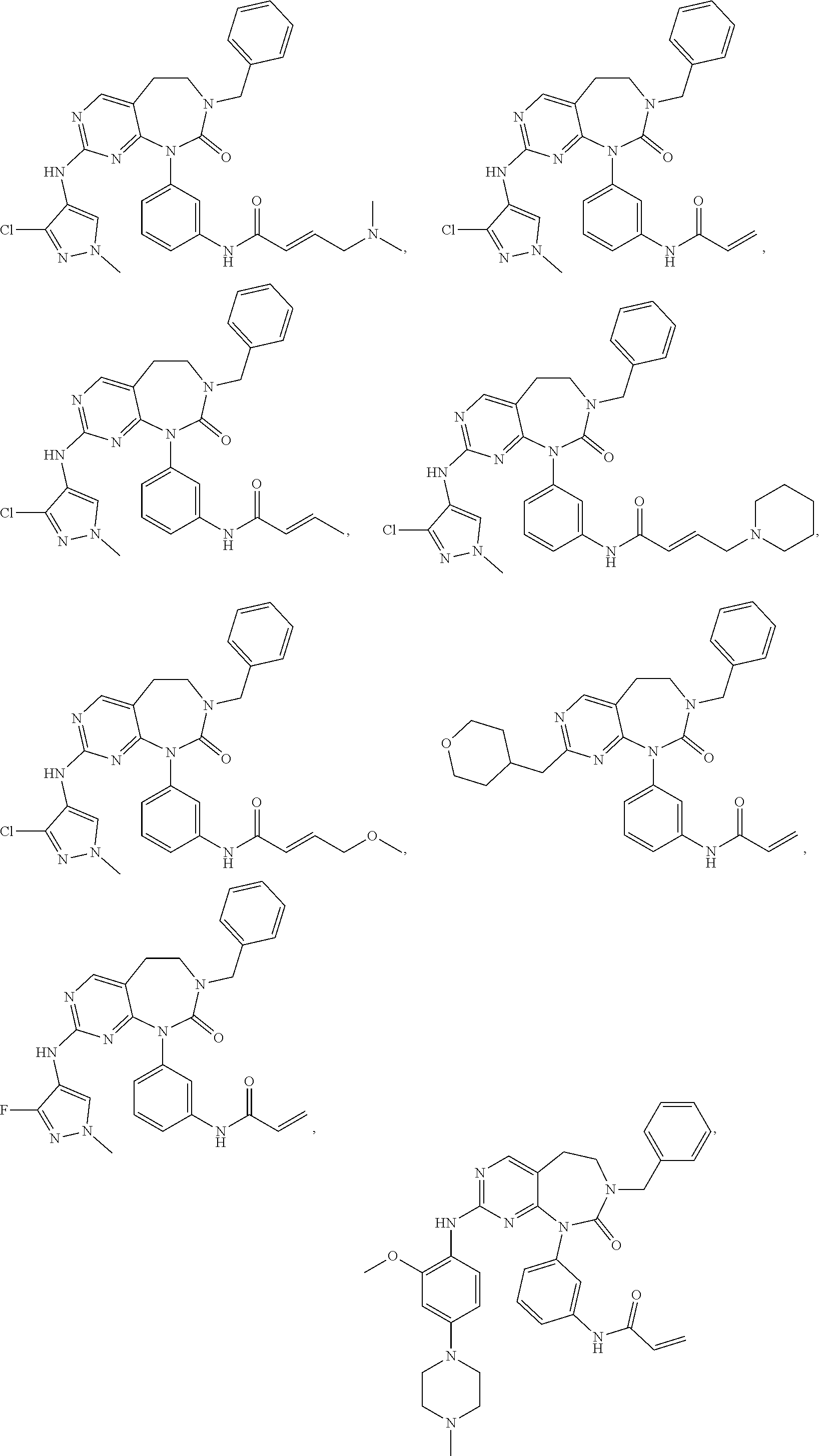

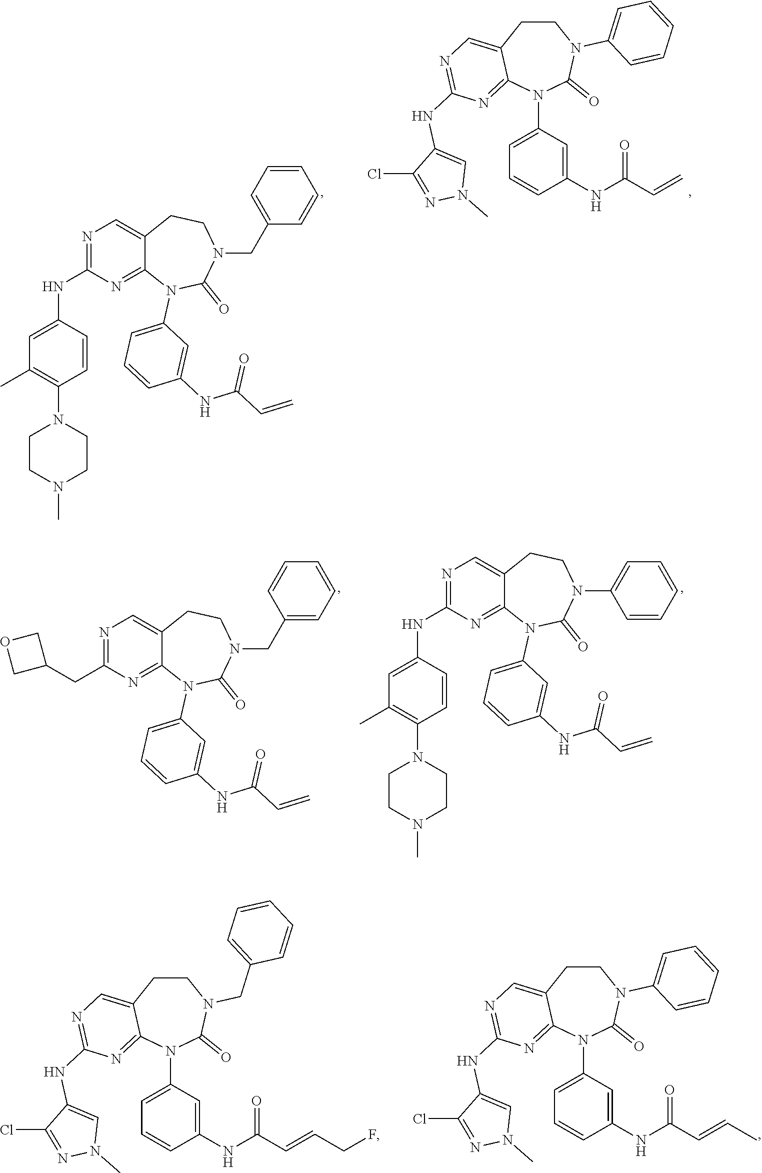 [SMILES: C=CC(=O)Nc1cccc(N2C(=O)N(Cc3ccccc3)CCc3cnc(Nc4ccc(N5CCN(C)CC5)c(C)c4)nc32)c1 ; C=CC(=O)Nc1cccc(N2C(=O)N(c3ccccc3)CCc3cnc(Nc4cn(C)nc4Cl)nc32)c1 ; C=CC(=O)Nc1cccc(N2C(=O)N(Cc3ccccc3)CCc3cnc(CC4COC4)nc32)c1 ; C=CC(=O)Nc1cccc(N2C(=O)N(c3ccccc3)CCc3cnc(Nc4ccc(N5CCN(C)CC5)c(C)c4)nc32)c1 ; Cn1cc(Nc2ncc3c(n2)N(c2cccc(NC(=O)/C=C/CF)c2)C(=O)N(Cc2ccccc2)CC3)c(Cl)n1 ; C/C=C/C(=O)Nc1cccc(N2C(=O)N(c3ccccc3)CCc3cnc(Nc4cn(C)nc4Cl)nc32)c1]

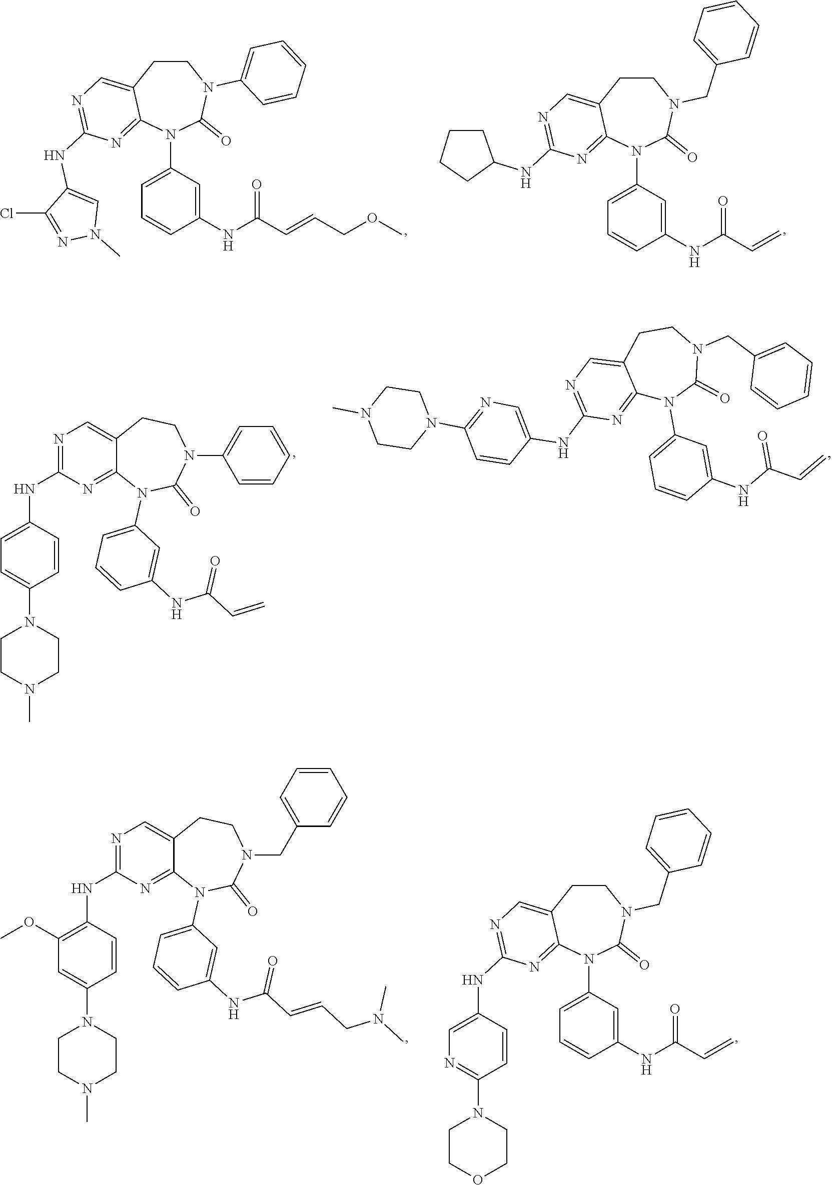

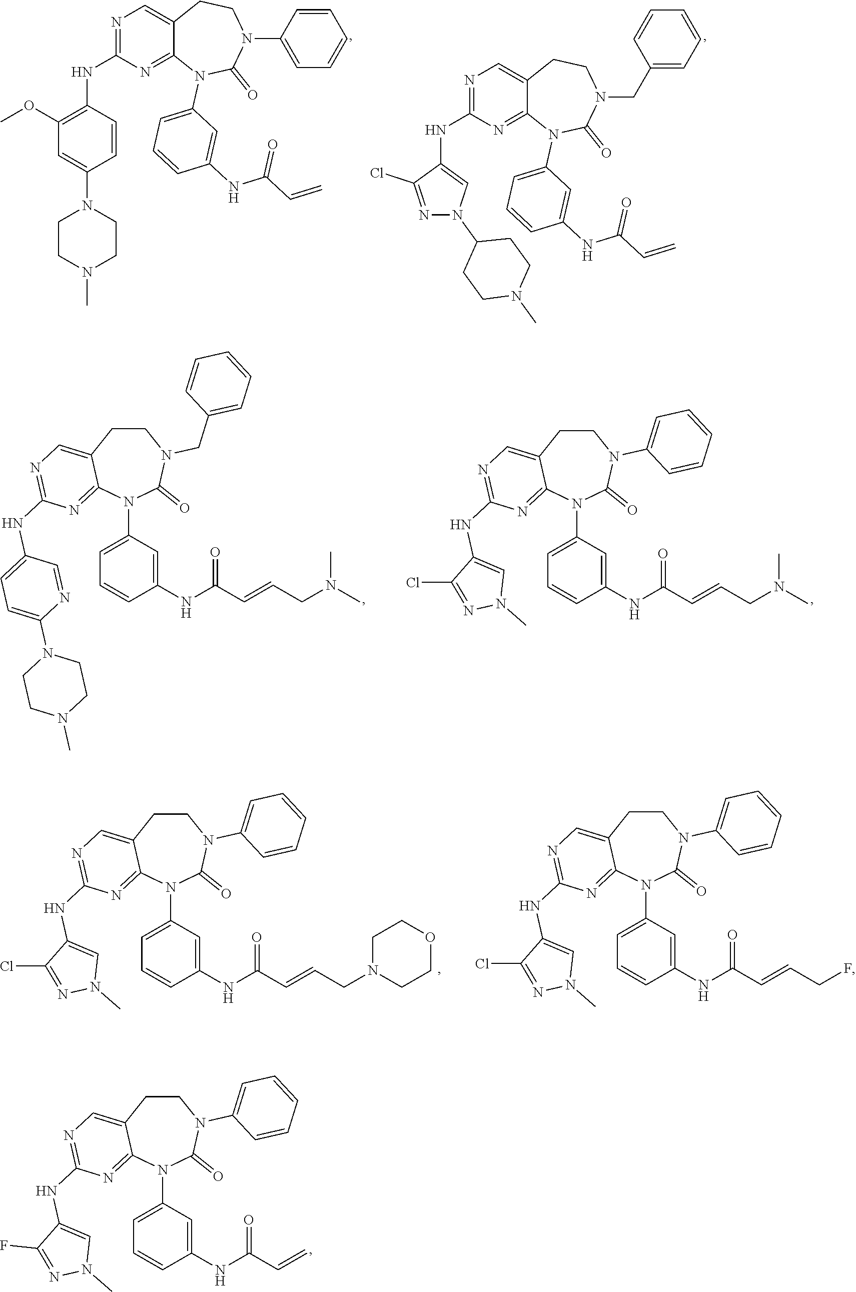

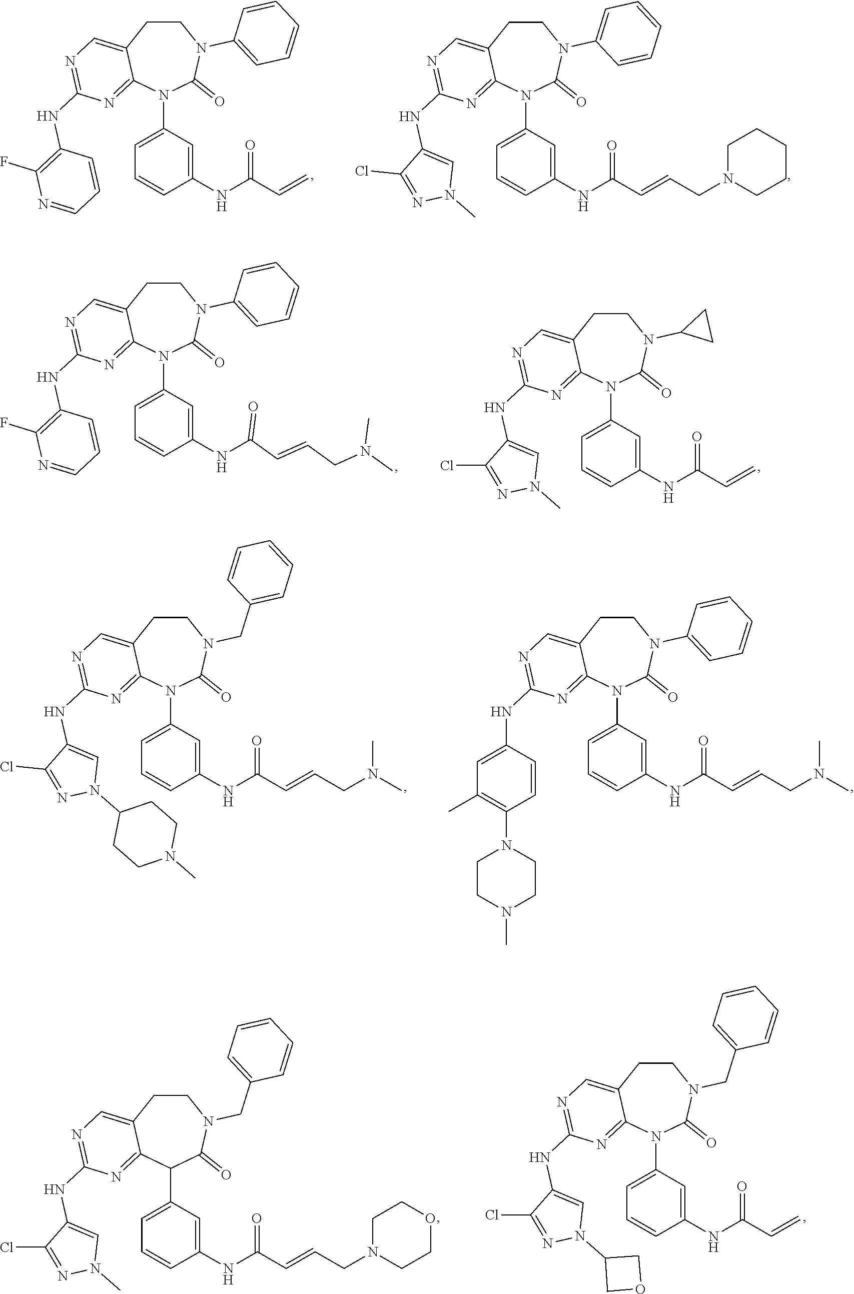

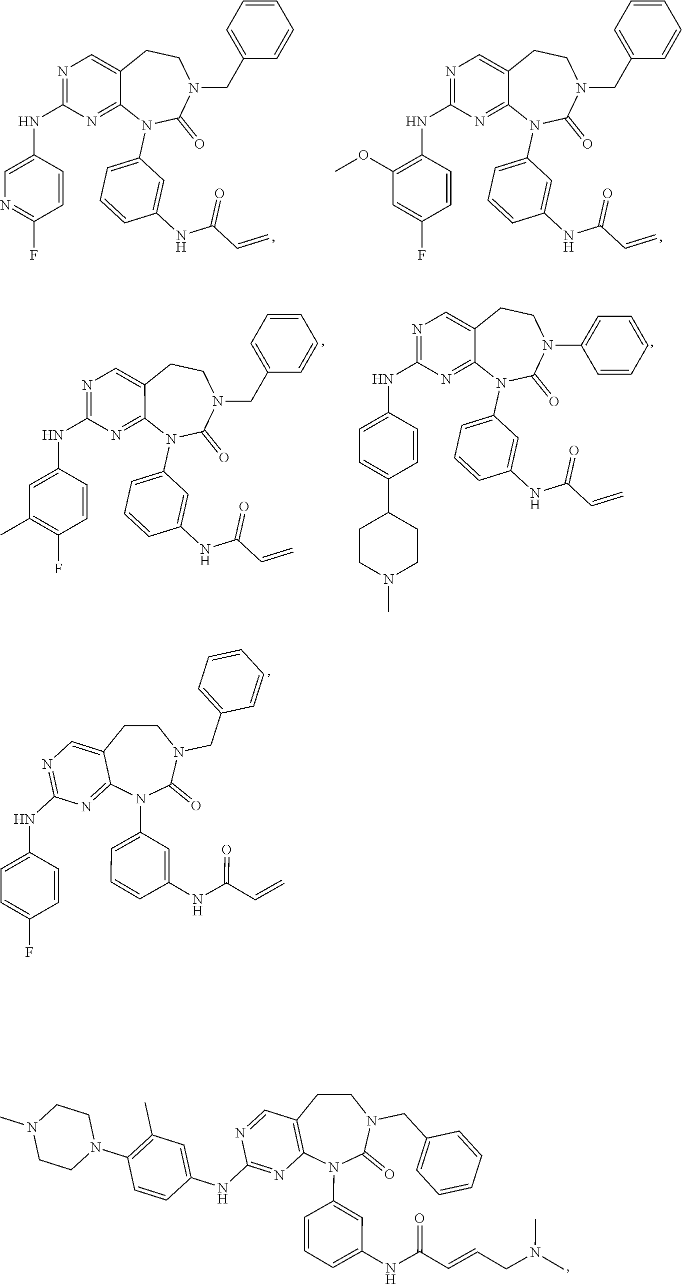

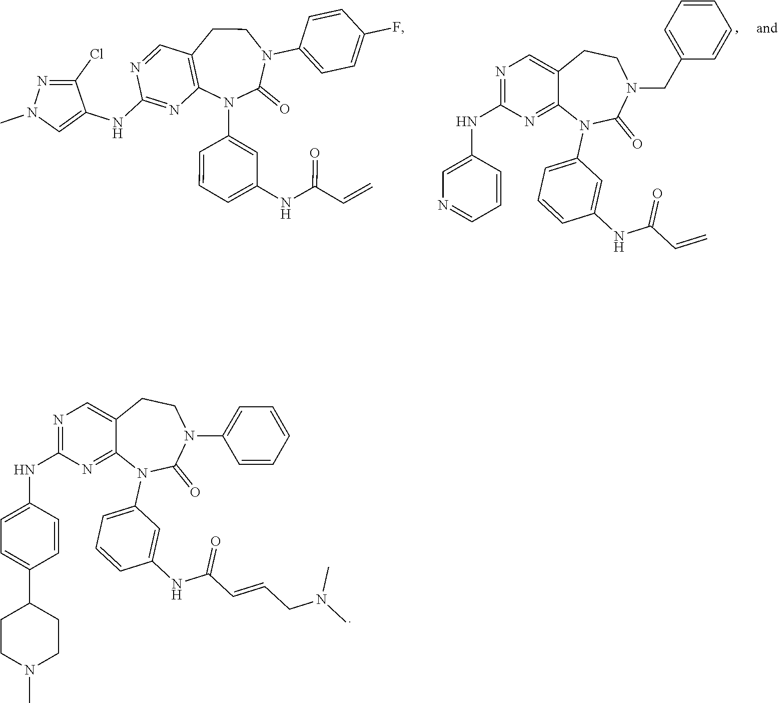 [SMILES: C=CC(=O)Nc1cccc(N2C(=O)N(c3ccc(F)cc3)CCc3cnc(Nc4cn(C)nc4Cl)nc32)c1 ; C=CC(=O)Nc1cccc(N2C(=O)N(Cc3ccccc3)CCc3cnc(Nc4cccnc4)nc32)c1 ; CN(C)C/C=C/C(=O)Nc1cccc(N2C(=O)N(c3ccccc3)CCc3cnc(Nc4ccc(C5CCN(C)CC5)cc4)nc32)c1]

64. A pharmaceutical composition comprising a compound of claim 1, or a pharmaceutically acceptable salt thereof, and a pharmaceutically acceptable carrier.

65-87. (canceled)

88. A method of treating cancer in a subject in need thereof, comprising administering to the subject a therapeutically effective amount of a compound of claim 1, or a pharmaceutically acceptable salt thereof.

89. The method of claim 88, wherein the cancer is selected from bladder cancer, prostate cancer, breast cancer, cervical cancer, colorectal cancer, endometrial cancer, gastric cancer, glioblastoma, head and neck cancer, lung cancer, and non-small cell lung cancer.

90-105. (canceled)
